(19) United States
(12) Patent Application Publication
(10) Pub. No.: US 2026/0263579 A1
(43) Pub. Date: Sep. 10, 2026

(54) IMMUNOGENIC COMPOSITION

(71) Applicant: GLAXOSMITHKLINE BIOLOGICALS SA, Rixensart (BE)

(72) Inventors: Agnese MARCELLI, Siena (IT); Malte MEPPEN, Siena (IT); Daniela STRANGES, Siena (IT)

(73) Assignee: GLAXOSMITHKLINE BIOLOGICALS SA, Rixensart (BE)

(21) Appl. No.: 18/869,582

(22) PCT Filed: May 30, 2023
(86) PCT No.: PCT/EP2023/064448

§ 371 Date: Nov. 26, 2024

(30) Foreign Application Priority Data
Jun. 1, 2022 (GB) 2208089.9

(51) Int. Cl. A61K 39/095; A61K 39/00; A61P 31/04

(52) U.S. Cl. CPC A61K 39/095; A61P 31/04; A61K 2039/55505; A61K 2039/70

(57) ABSTRACT

The present invention relates to an improved immunogenic composition against N. meningitidis serogroups A, C, W135 and Y in solid form, and to a reconstituted vaccine against N. meningitidis serogroups A, B, C, W135, Y obtained by reconstitution with a liquid immunogenic composition of Men B antigens. Kits and methods for the prevention and treatment of meningococcal infection and disease with the immunogenic composition or the reconstituted vaccine are also provided.

### TECHNICAL FIELD

This invention is in the field of vaccines. More particularly, it relates to an immunogenic composition against Neisseria meningitidis serogroups A, C, W135, and Y (in the following also referred to as “composition MenACWY”) in a solid form, and to a reconstituted vaccine composition against N. meningitidis serogroups A, B, C, W135, and Y (in the following also referred to as “composition ABCWY”) obtained by reconstitution of the present immunogenic composition against N. meningitidis serogroups A, C, W, and Y in a solid form into a liquid formulation of an immunogenic composition against N. meningitidis serogroup B. Kits and methods for immunizing a mammal against meningococcal infection and disease caused by the bacterial pathogen N. meningitidis, in particular of the infection and disease caused by serogroups A, B, C, W, and Y of this pathogen, with the above said reconstituted vaccine are also provided.

### BACKGROUND

Invasive meningococcal disease (IMD) is caused by the bacterial pathogen Neisseria meningitidis, and is a very serious disease with approximately 10% of mortality, often causing life-long disabilities, even when appropriate antibiotics and supportive therapy is administered.

Based on the organism's capsular polysaccharide, several serogroups of N. meningitidis have been identified: among these, the main serogroups associated to IMD worldwide are Men A, B, C, W135, and Y. Men A is the pathogen most implicated in epidemic disease in the sub-Saharan Africa. Men B and C are responsible for the vast majority of the cases in a number of regions including Canada, the United States, Australia, New Zealand and Europe; while Men W135 and Y are responsible for the rest of the cases in the United States and other developed countries.

There are vaccines currently in use that have been designed to immunize against serogroups A, C, W135 and Y of Neisseria meningitidis like GSK's MENVEO.

MENVEO comprises capsular saccharides of the meningococcal serogroups A, C, W135, and Y that are conjugated to a non-toxic mutant of diphtheria toxin as carrier protein, CRM197. The product on the market consists of a liquid formulation of the CRM197-conjugated MenCWY capsular saccharides for reconstitution of the solid, lyophilized MenA capsular saccharide conjugate.

The vaccine products marketed under the trade names MENACTRA, MENQUADFI and NIMENRIX also contain conjugated capsular saccharide antigens from each of serogroups Y, W135, C and A.

As far as Men B is concerned, there are currently two licensed vaccines that have been designed to immunize against serogroup B meningococcus: GSK's BEXSERO and Pfizer's TRUMENBA.

TRUMENBA contains two lipidated Men B fHbp antigens (v1.55 and v3.45) adsorbed on aluminum phosphate, while BEXSERO (also known generically, and referred to herein, as 4CMenB) contains a preparation of outer membrane vesicles (OMVs) from the epidemic strain of group B Meningococcal NZ98/254 together with five meningococcal antigens: Neisserial Heparin Binding protein A (NHBA), factor H binding protein (fHbp) variant 1.1, Neisserial adhesion protein A (NadA), and accessory proteins GNA1030 and GNA2091. Four of these antigens are present as fusion proteins (an NHBA-GNA1030 fusion protein and a GNA2091-fHbp fusion protein). The four antigens are adsorbed on aluminium hydroxide. 4CMenB is described in literature (for example, see Bai et al. (2011) Expert Opin Biol Ther. 11:969-85, Su & Snape (2011) Expert Rev Vaccines 10:575-88). The terms “BEXSERO” and “4CMenB” are used interchangeably herein.

fHbp (also known interchangeably in the art as genome-derived Neisseria antigen (GNA) 1870, LP2086 and protein ‘741’) binds to human factor H (hfH), which is a large (180 kDa) multi-domain soluble glycoprotein, consisting of 20 complement control protein (CCP) modules connected by short linker sequences. hfH circulates in human plasma and regulates the Alternative Pathway of the complement system. Functional binding of fHbp to hfH relies predominantly on CCP modules (or domains) 6-7 of hfH, and enhances the ability of the bacterium to resist complement-mediated killing. Therefore, expression of fHbp enables survival in ex vivo human blood and serum.

As different fHbp classification schemes have been proposed, a dedicated database is available with a unified fHbp nomenclature for the assignment of new sub-variants: (http)://neisseria.org/nm/typing/fhbp (also as (https)://pubmlst.org/neisseria/fHbp/).

fHbp has been classified into three (main) variants 1, 2 and 3, which were further divided into sub-variants fHbp-1.x, fHbp-2.x and fHbp-3.x, where x denotes the specific peptide sub-variant. In contrast to v2 and v3, fHbp v1 is highly heterogeneous and contains several subvariants. In a different nomenclature scheme, the sub/variants are grouped into subfamily A (corresponding to variants 2 and 3) and subfamily B (corresponding to variant 1) based on sequence diversity.

BEXSERO is predicted to provide broad coverage against Men B strains circulating worldwide (Medini D et al., Vaccine 2015; 33:2629-2636; Vogel U et al. Lancet Infect Dis 2013; 13:416-425; Křížová et al., Epidemiol Mikrobiol Imunol 2014; 63:103-106; Tzanakaki G et al. BMC Microbiol 2014; 14:111; Wasko I et al. Vaccine 2016; 34:510-515; 6. Simões M J et al. PLoS ONE 12(5): e0176177; and Parikh S R et al. Lancet Infect Dis 2017; 17:754-62). Furthermore, following the introduction of BEXSERO into the UK national infant immunization program in September 2015, data at 10 months showed 83% vaccine efficacy on all Men B strains after two doses (Parikh S R et al., Lancet 2016; 388:2775-82).

Recently, improved serogroup B meningococcus immunogenic compositions have been disclosed in WO 2020/030782, with mutated fHbp polypeptides and fusion proteins comprising these mutated polypeptides. These immunogenic compositions retain the efficacy of BEXSERO but also have an improved coverage against meningococcal strains carrying fHbp variants, in particular fHbp v2 and v3 variants, and strains carrying some v1 sub-variants. These compositions are heptavalent compositions after the fact that seven MenB antigens are contained therein: outer membrane vesicles (OMVs) from the epidemic strain of group B Meningococcal NZ98/254 with PorA and PorB Outer Membrane Proteins (OMPs), Neisserial Heparin Binding protein A (NHBA; 287), factor H binding protein (fHbp; 741), Neisserial adhesion protein A (NadA; 961), accessory proteins GNA1030 (953) and GNA2091 (936), and 231.13 fHbp fusion polypeptide (741 231.13). As for Bexsero, four of these antigens are present as fusion proteins: NHBA-GNA1030 fusion protein (287-953) and GNA2091-fHbp (936-741) fusion protein.

In fact, bactericidal activity is variant specific; antibodies raised against one variant are not necessarily cross-protective against other variants, although some cross-reactivity has been described between fHbp v2 and v3 (Masignani V et al., J Exp Med 2003; 197:789-799). Antibodies raised against sub-variant fHbpv1.1, included in the 4CMenB vaccine, are highly cross-reactive with the most frequently occurring fHbp v1 sub-variants but are less cross-reactive with v1 sub-variants that are most distantly related to v1.1. Furthermore, antibodies raised against sub-variant fHbpv1.1 included in the 4CMenB vaccine are poorly cross-reactive with fHbp v2 and v3 (Brunelli B et al., Vaccine 2011; 29:1072-1081), while the improved vaccine comprising the mutated fHbp polypeptides provides a coverage against the variants too.

No vaccines are on the market up to date that have, in a single drug product, a coverage against meningococcal infection and disease caused by all main serogroups A, B, C, W135 and Y of N. meningitidis. Such a co-immunisation with a single vaccine composition in which the different immunogens are admixed offers to vaccinees the advantage of receiving a reduced number of injections, which can lead to the clinical advantage of increased compliance.

Besides this unmet need of having available such a product, there is also the need that this product represents an effective formulation, able to maintain the single antigens integrity and immunogenicity of the components and to guarantee at the same time the required safety, stability and easiness of use.

### SUMMARY OF THE INVENTION

An experimental study has been carried out by the inventors on a solid formulation for an improved immunogenic composition against N. meningitidis serogroups A, C, W135, and Y, wherein all saccharides of the four different serogroups are formulated in a solid composition, that may be reconstituted with a liquid before use as a vaccine.

Also, this study has unexpectedly shown that a matrix of pharmaceutically acceptable excipients, illustrated in the following detailed description, for the solid formulation of the Men A, C, W135, Y antigens is effective to guarantee the stability of the formulation during long-term storage. At the same time, when a liquid immunogenic composition against N. meningitidis serogroup B is used for the reconstitution of this solid formulation, it has shown that this formulation reduces the negative effect that reconstitution has on the adsorption of Men B antigens onto an adsorbing agent, e.g. a compound containing aluminum. With the present solid formulation of the Men A, C, W135, Y antigens, an improved Men ABCW135Y reconstituted vaccine, comprising for instance seven MenB antigens, can therefore be obtained.

More in particular, the herein described solid (e.g. freeze-dried) form of the composition against N. meningitidis serogroups A, C, W135, and Y, minimizes the effect of desorption of Men B antigens from an adsorbing agent caused by the reconstitution of the solid Men A, C, W135, and Y composition with a liquid formulation of Men B antigens adsorbed onto an aluminum adsorbing agent, without impairing the stability of the solid composition during long-term storage.

According to an aspect of this invention, there is provided an immunogenic composition against N. meningitidis serogroups A, C, W135, and Y, comprising a composition of capsular polysaccharide antigens of the meningococcal serogroups A, C, W135, and Y conjugated to carrier protein(s) and one or more pharmaceutically acceptable excipients, wherein this composition is in a solid form (e.g. freeze-dried) and said excipients comprise one or more bulking agents in amount ≤30 mg per unit dose of the composition of polysaccharide antigens in solid form.

A second aspect of this invention provides a reconstituted vaccine composition against N. meningitidis serogroups A, B, C, W135, and Y obtainable by reconstitution of the above said immunogenic composition against N. meningitidis serogroups A, C, W135, and Y in solid form with a liquid formulation of an immunogenic composition against N. meningitidis serogroup B comprising one or more Men B antigens and an adsorbing agent for these antigens, preferably with a heptavalent MenB liquid formulation.

A third aspect of this invention provides a kit comprising (i) a first container comprising the above said liquid formulation of an immunogenic composition against N. meningitidis serogroup B, preferably a heptavalent MenB formulation; and (ii) a second container comprising the above said immunogenic composition against N. meningitidis serogroups A, C, W135, and Y in a solid form.

A fourth aspect of this invention provides a method for the preparation of a vaccine composition against N. meningitidis serogroups A, B, C, W135, and Y comprising the step of reconstituting the above said immunogenic composition in solid form against N. meningitidis serogroups A, C, W135, and Y with the above said liquid formulation of an immunogenic composition against N. meningitidis serogroup B.

A fifth aspect of this invention is the above said immunogenic composition against N. meningitidis serogroups A, C, W135, and Y in solid form, or the reconstituted vaccine composition for use in immunizing a mammal, preferably a human, against meningococcal infection and disease caused by N. meningitidis serogroups A, B, C, W135, and Y.

A sixth aspect of this invention is the use of the above said immunogenic composition against N. meningitidis serogroups A, C, W135, and Y in solid form or of the above said reconstituted vaccine in the manufacture of a medicament for the prevention or treatment of meningococcal infections or diseases caused by N. meningitidis serogroups A, B, C, W135, and Y.

A seventh aspect of this invention provides a method of prevention or treatment of meningococcal infections or diseases caused by N. meningitidis serogroups A, B, C, W135, and Y, comprising administering to a subject in need thereof a reconstituted vaccine of the invention.

An eighth aspect of this invention provides a pre-lyophilization solution comprising a composition of capsular polysaccharide antigens of the meningococcal serogroups A, C, W135, and Y conjugated to carrier protein(s), one or more pharmaceutically acceptable excipients, and an aqueous solution of a buffering agent.

### DESCRIPTION OF THE FIGURES

FIG. 1 shows the RM % determined with the Karl Fischer method, as described in the following Example 1, in lyophilised compositions of this invention stored at 2-8° C., at time zero and at different times up to 2.5 years from the production day.

FIG. 2 shows the RM % determined with the Karl Fischer method, as described in the following Example 1, in lyophilised compositions of this invention incubated at 25° C./60% RH approximately 2 weeks after the production day, measured at different times up to 6 months.

FIG. 3 shows the RM % determined with the Karl Fischer method, as described in the following Example 1, in lyophilised compositions of this invention incubated at 37° C./75% RH approximately 2 weeks after the production day, measured at different times up 2 months.

FIG. 4 shows the adsorption analyses results for the MenB antigens performed by SDS-PAGE on a gel. Lane 1: molecular weight standard. Reference standard for each of the 3 recombinant proteins “std rMenB”) at 15%, 10% and 5% recombinant protein were loaded in lanes 2-4. Samples of MenABCWY vaccine immediately following (box in the center) and 24-hrs. after reconstitution (box on the right) were loaded with or with 5% recombinant protein spike for the combined lot permutations indicated in lanes 5-16. Lane 17 is loaded with 2.5 μg OMV standard as control. Three lots of Bexsero (B1, B2 and B3) and two lots of MenACWY lyophilized product (A1 and A2) were used.

FIG. 5 shows a RP-UPLC chromatogram of Bexsero lot B2 before and immediately after reconstitution (T=0) with lyophilised MenACW135Y lots A1 and A2.

### DETAILED DESCRIPTION OF THE INVENTION

### General

The term “comprising” encompasses “including” as well as “consisting” e.g. a composition “comprising” X may consist exclusively of X or may include something additional e.g. X+Y. References to “comprising” (or “comprises”, etc.) may optionally be replaced by references to “consisting of” (or “consists of”, etc.). The term “consisting essentially of” limits the scope of a claim to the specified materials or steps “and those that do not materially affect the basic and novel characteristic(s)” of the claimed invention.

The term “about” in relation to a numerical value x is optional and means, for example, x±10%.

By the term “unit dose”, referred to the present immunogenic composition in solid form of MenACWY polysaccharides is meant herein the amount of immunogenic composition administered to a patient in a single dose. In general, a unit dose of the immunogenic composition according to this invention may comprise an amount from 0.1 to 100 μg of each polysaccharide, and typically an amount from 2 to 20 μg of each polysaccharide, these values being measured as polysaccharides. An exemplary unit dose of the Men ACWY composition comprises 10 μg of the MenA polysaccharide and 5 μg of each of the Men C, Men W and Men Y polysaccharides.

The word “substantially” does not exclude “completely” e.g. a composition which is “substantially free” from Y may be completely free from Y. Where necessary, the word “substantially” may be omitted from the definition of the invention.

Where the disclosure concerns an “epitope”, this epitope may be a B-cell epitope and/or a T-cell epitope but will usually be a B-cell epitope. Such epitopes can be identified empirically (e.g. using PEPSCAN (e.g. see Geysen et al. (1984) PNAS USA 81:3998-4002 and Carter (1994) Methods Mol Biol 36:207-23) or similar methods), or they can be predicted (e.g. using the Jameson-Wolf antigenic index (Jameson, B A et al. 1988, CABIOS 4(1):181-186), matrix-based approaches (Raddrizzani & Hammer (2000) Brief Bioinform 1(2):179-89), MAPITOPE (Bublil et al. (2007) Proteins 68(1):294-304), TEPITOPE (De Lalla et al. (1999) J. Immunol. 163:1725-29 and Kwok et al. (2001) Trends Immunol 22:583-88), neural networks (Brusic et al. (1998) Bioinformatics 14(2):121-30), OptiMer & EpiMer (Meister et al. (1995) Vaccine 13(6):581-91 and Roberts et al. (1996) AIDS Res Hum Retroviruses 12(7):593-610), ADEPT (Maksyutov & Zagrebelnaya (1993) Comput Appl Biosci 9(3):291-7), Tsites (Feller & de la Cruz (1991) Nature 349 (6311): 720-1), hydrophilicity (Hopp (1993) Peptide Research 6:183-190), or antigenic index (Welling et al. (1985) FEBS Lett. 188:215-218)). Epitopes are the parts of an antigen that are recognized by and bind to the antigen binding sites of antibodies or T-cell receptors, and they may also be referred to as “antigenic determinants”.

The term “suspension” means a mixture in which solid particles are dispersed throughout a liquid, including throughout a liquid composition.

As used herein, references to “percentage sequence identity” between a query amino acid sequence and a subject amino acid sequence are understood to refer to the value of identity that is calculated using a suitable algorithm or software program known in the art to perform pairwise sequence alignment.

A query amino acid sequence may be described by an amino acid sequence identified in one or more claims herein. The query sequence may be 100% identical to the subject sequence, or it may include up to a certain integer number of amino acid alterations (e.g. point mutations, substitutions, deletions, insertions etc.) as compared to the subject sequence, such that the % identity is less than 100%. For example, the query sequence is at least 80, 85, 90, 95, 96, 97, 98, or 99% identical to the subject sequence.

Preferred alignment tools used to perform alignment and calculate percentage (%) sequence identity are local alignment tools, such as the Basic Local Alignment Search Tool (BLAST) algorithms. Software for performing BLAST analyses is publicly available through the National Centre for Biotechnology Information (www.ncbi.nlm.nih.gov). Alignment may be determined by the Smith-Waterman homology search algorithm using an affine gap search with a gap open penalty of 12 and a gap extension penalty of 2, BLOSUM matrix of 62. The Smith-Waterman homology search algorithm is disclosed in Smith & Waterman (1981) Adv. Appl. Math. 2:482-489. Other preferred alignment tools are Water (EMBOSS) and Marcher (EMBOSS). Alternatively, preferred alignment tools used to perform alignment and calculate percentage (%) sequence identity are best fit alignment tools, such as GENEPAST, also known as KERR algorithm.

In order to calculate percent identity, the query and subject sequences may be compared and aligned for maximum correspondence over a designated region (e.g. a region of at least about 40, 45, 50, 55, 60, 65 or more amino acids in length, and can be up to the full length of the subject amino acid sequence). Said designated region must include the region of the query sequence comprising any specified point mutations in the amino acid sequence. Alternatively, percentage sequence identity may be calculated over the “full length” of the subject sequence. Any N-terminal or C-terminal amino acid stretches that may be present in the query sequence, such as signal peptides or leader peptide or C-terminal or N-terminal tags, should excluded from the alignment.

The term “fragment” referred to polypeptide sequences means that the polypeptide is a fraction of a full-length protein. As used herein, a fragment of a mutant polypeptide also comprises the mutation(s). Fragments may possess qualitative biological activity in common with the full-length protein, for example, an “immunogenic fragment” contains or encodes one or more epitopes, such as immunodominant epitopes, that allows the same or similar immune response to be raised to the fragment as is raised to the full-length sequence. Polypeptide fragments generally have an amino (N) terminus portion and/or carboxy (C) terminus portion deleted as compared to the native protein, but wherein the remaining amino acid sequence of the fragment is identical to the amino acid sequence of the native protein. Polypeptide fragments may contain, for example: about 8, 10, 12, 14, 16, 18, 20, 22, 24, 26, 28, 30, 32, 24, 26, 28, 40, 45, 50, 55, 60, 70, 80, 90, 100, 150, 200, 240, 241, 242, 243, 244, 245, 246, 247, 248, 249, 250, 251, 252, 253, 254, 255, 256, 257, 258, 259, 260, 261, 262 contiguous amino acids, including all integers in between, of a reference polypeptide sequence, for example between 50 and 260, 50 and 255, 50 and 250, 50 and 200, 50 and 150 contiguous amino acids of a reference polypeptide sequence. The term fragment explicitly excludes full length fHbp polypeptides and mature lipoproteins thereof.

After serogroup, meningococcal classification includes serotype, serosubtype and then immunotype, and the standard nomenclature lists serogroup, serotype, serosubtype, and immunotype, each separated by a colon e.g. B:4:P1.15:L3,7,9. Within serogroup B, some lineages cause disease often (hyperinvasive), some lineages cause more severe forms of disease than others (hypervirulent), and others rarely cause disease at all. Seven hypervirulent lineages are recognized, namely subgroups I, III and IV-1, ET-5 complex, ET-37 complex, A4 cluster and lineage 3. These have been defined by multilocus enzyme electrophoresis (MLEE), but multilocus sequence typing (MLST) has also been used to classify meningococci. The four main hypervirulent clusters are ST32, ST44, ST8 and ST11 complexes.

References herein to “enhanced stability” or “higher stability” or “increased stability” mean that the mutant polypeptides disclosed herein have a higher relative thermostability (in kcal/mol) as compared to a non-mutant (wild-type) polypeptide under the same experimental conditions. The stability enhancement can be assessed using differential scanning calorimetry (DSC), for example as discussed in Bruylants et al. (Differential Scanning calorimetry in Life Sciences: Thermodynamics, Stability, Molecular Recognition and Application in Drug Design, 2005 Curr. Med. Chem. 12:2011-2020) and calorimetry Sciences Corporation's “Characterizing Protein stability by DSC” (Life Sciences Application Note, Doc. No. 2021102136 February 2006) or by differential scanning fluorimetry (DSF). An increase in stability may be characterized as an at least about 5° C. increase in thermal transition midpoint (Tₘ), as assessed by DSC or DSF. See, for example, Thomas et al., Effect of single-point mutations on the stability and immunogenicity of a recombinant ricin A chain subunit vaccine antigen, 2013 Hum. Vaccin. Immunother. 9(4):744-752.

By the expression “matrix of pharmaceutically acceptable excipients” or “matrix of excipients” is meant the ensemble of pharmaceutically acceptable excipients in the solid lyophilized composition of the present invention.

### Meningococcus Serogroups a, C, W135 and Y

The present immunogenic composition against N. meningitidis serogroups A, C, W135, and Y, in a solid form (e.g. freeze-dried), comprises capsular saccharides of the meningococcal serogroups A, C, W135, and Y that are conjugated to carrier protein(s). The capsular saccharides of each of these four serogroups ACWY of N. meningitidis are well characterized.

The capsular saccharide of serogroup A meningococcus is a homopolymer of (α1→6)-linked N-acetyl-D-mannosamine-1-phosphate, with partial O-acetylation in the C3 and C4 positions. The acetyl groups can be replaced with blocking groups to prevent hydrolysis (see, e.g., WO03/080678, which is incorporated herewith by reference), and such modified saccharides are still serogroup A capsular saccharides as disclosed herein.

The serogroup C capsular saccharide is a homopolymer of (α2→9)-linked sialic acid (N-acetyl neuraminic acid (“NeuNAc”)). Most serogroup C strains have O-acetyl groups at C-7 and/or C-8 of the sialic acid residues, but about 15% of clinical isolates lack these O-acetyl groups. The saccharide structure is written as →9)-NeupAc(7/8OAc)-(α2→.

The serogroup W135 saccharide is a polymer of sialic acid-galactose disaccharide units. Like the serogroup C saccharide, it has variable O-acetylation, but at sialic acid 7 and 9 positions. The structure is written as: →4)-D-Neup5Ac(7/9OAc)-α-(2→6)-D-Gal-α-(1→.

The serogroup Y saccharide is similar to the serogroup W135 saccharide, except that the disaccharide repeating unit includes glucose instead of galactose. Like serogroup W135, it has variable O-acetylation at sialic acid 7 and 9 positions. The serogroup Y structure is written as: →4)-D-Neup5Ac(7/9OAc)-α-(2→6)-D-Glc-α-(1→.

The capsular saccharides may be native capsular saccharides obtained from a meningococcal bacterium of the related serogroup. The native capsular saccharide may be modified by any method available to one with ordinary skills in the art so long as the capsular saccharide retains at least one epitope that elicits serum bactericidal antibodies. Exemplary modifications are detailed below. In addition to native and modified capsular saccharides, the capsular saccharides may be chemically synthesized as long as the synthesized compound (saccharide, saccharide analog, etc.) includes at least one epitope that elicits serum bactericidal antibodies that bind to capsular saccharides. All such native, modified and chemically synthesized capsular saccharides are within the scope of the meningococcal capsular saccharides disclosed herein. Exemplary modifications and chemical syntheses are described below.

The capsular saccharides in the vaccines may be O-acetylated as described above (e.g., with the same O-acetylation pattern as seen in native capsular saccharides), or they may be partially or totally de-O-acetylated at one or more positions of the saccharide rings, or they may be hyper-O-acetylated relative to the native capsular saccharides.

The capsular saccharides in the vaccines may be shorter than the native capsular saccharides seen in bacteria. Thus, the saccharides may be partially depolymerized, which typically occurs after purification but before conjugation. Depolymerization reduces the chain length of the saccharides up to the formation of saccharides of the desired size. A depolymerization method involves the use of hydrogen peroxide which may be added to a saccharide (e.g., to give a final H₂O₂ concentration of 1%), and the mixture is then incubated (e.g., at about 55° C.) until a desired chain length reduction has been achieved. Another depolymerization method involves acid hydrolysis (see, e.g., WO03/007985, which is incorporated herewith by reference). Other depolymerization methods are known to any skilled person. The capsular saccharides used in the vaccines may be obtainable by any of these depolymerization methods. Depolymerization can be used in order to provide an optimum chain length for immunogenicity and/or to reduce chain length for physical manageability of the saccharides. Native capsular saccharides are typically referred to as capsular polysaccharides while depolymerized capsular saccharides are typically referred to as capsular oligosaccharides.

In this present composition capsular saccharides may be used in the form of oligosaccharides. These are conveniently formed by fragmentation of purified capsular polysaccharide as described above, which will usually be followed by purification of the fragments of the desired size.

The vaccine products marketed under the trade names MENVEO, MENACTRA, MENQUADFI and NIMENRIX all contain conjugated capsular saccharide antigens from each of serogroups Y, W135, C and A.

In MENVEO (also known generically as Meningococcal (Groups A, C, Y, and W-135) Oligosaccharide Diphtheria CRM197 Conjugate Vaccine) each of the Men A, C, W135 and Y antigens is conjugated to a CRM197 carrier.

In MENACTRA (also known generically as Meningococcal (Groups A, C, Y and W-135) Polysaccharide Diphtheria Toxoid Conjugate Vaccine) each of the A, C, W135 and Y antigens is conjugated to a diptheria toxoid carrier.

In MENQUADFI (also known generically as Meningococcal (Groups A, C, Y and W-135) Polysaccharide Conjugate Vaccine) each of the A, C, W135 and Y antigens is conjugated to a tetanus toxoid carrier.

In NIMENRIX (also known generically as Meningococcal polysaccharide groups A, C, W135 and Y conjugate vaccine) each of the Men A, C, W135 and Y antigens is conjugated to a tetanus toxoid carrier.

In preferred embodiments, the immunogenic composition in solid form of this invention comprises the Men A, C, W135 and Y antigen conjugates which are present in MENVEO, the Men A, C, W135 and Y antigen conjugates, which are present in MENACTRA, the Men A, C, W135 and Y antigen conjugates, which are present in MENQUADFI, or the Men A, C, W135 and Y antigen conjugates which are present in NIMENRIX. In an embodiment, the immunogenic composition in solid form of this invention comprises the Men A, C, W135 and Y antigen conjugates, wherein the capsular saccharides and/or oligosaccharides thereof are conjugated to CRM₁₉₇ carrier, including recombinant CRM₁₉₇ carrier (rCRM₁₉₇), a tetanus toxoid carrier (TT), or a diphtheria toxoid carrier (DT). In an embodiment, the immunogenic composition in solid form of this invention comprises the Men A, C, W135 and Y antigen conjugates, wherein the capsular saccharides and/or oligosaccharides thereof are conjugated to CRM₁₉₇ carrier, including recombinant CRM₁₉₇ carrier (rCRM₁₉₇), or a tetanus toxoid carrier (TT).

Preferably, the immunogenic composition in solid form of this invention comprises the Men A, C, W135 and Y antigen conjugates, wherein the capsular saccharides and/or oligosaccharides thereof are conjugated to CRM₁₉₇ carrier, including recombinant CRM₁₉₇ carrier (rCRM₁₉₇).

### Meningococcus Serogroup B

The above said immunogenic composition of Men ACW135Y antigens in solid form can be reconstituted with a liquid formulation of an immunogenic composition against N. meningitidis serogroup B, comprising at least a Men B antigen that may or may not be adsorbed to an adsorbing agent, e.g. an aluminum salt. Thus, the present invention provides a reconstituted vaccine against N. meningitidis serogroups A, B, C, W135, and Y comprising the immunogenic composition against N. meningitidis serogroups A, C, W135, and Y in solid form, reconstituted with a liquid formulation of an immunogenic composition against N. meningitidis serogroup B comprising one or more Men B antigens and an adsorbing agent for said antigens.

The MenB composition in a liquid formulation may comprise one or more antigens, as disclosed for instance in WO2004/032958, WO2016/008960, and WO2020/030782, all of which are incorporated herewith by reference.

In an embodiment the liquid Men B composition comprises more than two Men B antigens.

In another embodiment the liquid Men B composition comprises at least the GNA2091-fHbp (936-741) fusion protein, alone or in combination with one or more other Men B antigens.

This Men B composition in liquid formulation may comprise for instance two or more antigens selected from: a meningococcal NHBA antigen, a meningococcal NadA antigen, a meningococcal fHbp antigen and meningococcal outer membrane vesicles (OMVs). The Men B composition in liquid formulation may comprise for instance a meningococcal NadA antigen, a meningococcal fHbp antigen and meningococcal outer membrane vesicles (OMVs). The Men B composition in liquid formulation may comprise for instance a meningococcal NHBA antigen, a meningococcal NadA antigen, a meningococcal fHbp antigen and meningococcal outer membrane vesicles (OMVs).

The Men B composition in liquid formulation may comprise for instance two or more fHbp antigens. The Men B composition in liquid formulation may comprise for instance two or more meningococcal fHbp antigens. The Men B composition in liquid formulation may comprise for instance a meningococcal NadA antigen, two or more meningococcal fHbp antigens and meningococcal outer membrane vesicles (OMVs). The Men B composition in liquid formulation may comprise for instance a meningococcal NHBA antigen, a meningococcal NadA antigen, two or more meningococcal fHbp antigens and meningococcal outer membrane vesicles (OMVs).

In an aspect of this invention, the liquid formulation of Men B antigens may further comprise a fusion polypeptide of meningococcal fHbp polypeptides. The Men B composition in liquid formulation may comprise for instance a meningococcal NHBA antigen, a meningococcal NadA antigen, a fusion polypeptide of meningococcal fHbp polypeptides and meningococcal outer membrane vesicles (OMVs).

The present invention also provides a reconstituted vaccine comprising for instance two or more antigens selected from: a meningococcal NHBA antigen, a meningococcal NadA antigen, a meningococcal fHbp antigen and meningococcal outer membrane vesicles (OMVs). The present invention also provides a reconstituted vaccine comprising for instance two or more meningococcal fHbp antigens. The present invention provides a reconstituted vaccine comprising a meningococcal NadA antigen, a meningococcal fHbp antigen, and meningococcal outer membrane vesicles (OMVs). The present invention provides a reconstituted vaccine comprising a meningococcal NadA antigen, two or more meningococcal fHbp antigens, and meningococcal outer membrane vesicles (OMVs). Thus, the present invention provides a reconstituted vaccine comprising a meningococcal NHBA antigen, a meningococcal NadA antigen, a meningococcal fHbp antigen, and meningococcal outer membrane vesicles (OMVs).

In a preferred embodiment, the meningococcal NHBA antigen and the meningococcal fHbp antigen of this immunogenic composition in liquid form are fusion proteins with meningococcal accessory proteins, e.g. with GNA1030 and GNA2091. Most preferably, the meningococcal NHBA antigen and the meningococcal fHbp antigen of this immunogenic composition are respectively an NHBA-GNA1030 fusion protein and a GNA2091-fHbp fusion protein.

Where the present immunogenic composition against N. meningitidis serogroup B comprises OMVs, these OMVs can be any proteoliposomic vesicle obtained by disruption of or blebbing from a meningococcal outer membrane to form vesicles therefrom that retain antigens from the outer membrane. Thus, this term includes, for instance, OMVs (sometimes referred to as ‘blebs’), microvesicles (MVs), ‘native OMVs’ (‘NOMVs’) extracted from cells using detergent-free methods, and detergent-extracted OMVs (dOMVs), such as OMVs extracted from cells using deoxycholate treatment.

The mass of OMVs is measured as the amount of total protein.

Preferred meningococcal OMVs comprise a PorA serotype 1.4. Preferably, the OMVs comprise a PorA variable region epitope 1.7-2 (VR1) and/or 1.4 (VR2). OMVs comprising both of these epitopes are more preferred (i.e. P1.7-2,4). OMVs obtained from strain NZ98/254 are particularly preferred.

In a most preferred embodiment, the immunogenic composition against N. meningitidis serogroup B in liquid form contains, as the OMV antigen, a preparation of OMV from the epidemic strain of group B Meningococcal NZ98/254, B: 4:P1.7b,4.

In a further preferred embodiment, the immunogenic composition against N. meningitidis serogroup B of the invention comprises five meningococcal antigens: NHBA (287; subvariant 1.2), fHbp (741; subvariant 1.1), NadA (961; subvariant 3.1), GNA1030 (953) and GNA2091 (936). Four of these antigens are present as fusion proteins (an NHBA-GNA1030 fusion protein (287-953) and a GNA2091-fHbp (936-741) fusion protein).

In one embodiment, the immunogenic composition of the invention against N. meningitidis serogroup B comprises the complete vaccine product 4CMenB, marketed under the trade name BEXSERO.

The polypeptides (or a subset thereof, e.g. non-OMV or soluble polypeptides) in a composition may be present at substantially equal masses i.e. the mass of each of them is within +5% of the mean mass of all the polypeptides in the composition (or the mean mass of the selected subset of polypeptides). For example, where the composition includes NHBA, fHbp and NadA, they may be present at substantially equal masses, e.g. at a mass ratio of a:b:c, where each of a, b & c is between 0.95 and 1.05.

### NHBA (Neisserial Heparin Binding Antigen)

NHBA was included in the published genome sequence for meningococcal serogroup B strain MC58 (Tettelin et al. (2000) Science 287:1809-1815) as gene NMB2132 (GenBank accession number GI: 7227388; SEQ ID NO: 4 herein).

References to NHBA herein include truncated variants of NHBA, wherein the N-terminus of the wild-type NHBA polypeptide sequence has been deleted up to and including its poly-glycine sequence (i.e. deletion of residues 1 to 24 in meningococcal strain MC58 (SEQ ID No. 4)). The resulting truncated variant is sometimes distinguished herein by the use of a ‘AG’ prefix. This deletion can enhance expression. The ‘AG’ variant of meningococcal NHBA is referred to herein as SEQ ID NO. 8.

Preferred NHBA antigens for use with the invention comprise an amino acid sequence: (a) having 70% or more identity (e.g. 70%, 75%, 80%, 85%, 90%, 91%, 92%, 93%, 94%, 95%, 96%, 97%, 98%, 99%, 99.5% or more) to SEQ ID NO: 4; and/or (b) comprising a fragment of at least ‘n’ consecutive amino acids of SEQ ID NO: 4, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 4. Particularly preferred NHBA antigens for use with the invention comprise the amino acid sequence of SEQ ID NO: 8.

A polypeptide including a neisserial NHBA antigen sequence can include that sequence alone, or it can be a fusion protein. One useful fusion partner for a NHBA sequence is the GNA1030 (953) polypeptide, which will normally be downstream of the NHBA sequence. Thus, the NHBA antigen can be present in a composition of the invention as a NHBA-GNA1030 fusion (e.g. SEQ ID NO: 9).

### NadA (Neisserial Adhesin A)

The NadA antigen was included in the published genome sequence for meningococcal serogroup B strain MC58 (Tettelin et al. (2000) Science 287:1809-1815) as gene NMB 1994 (GenBank accession number GI: 7227256; SEQ ID NO: 5 herein).

Preferred NadA antigens for use with the invention comprise an amino acid sequence: (a) having 70% or more identity (e.g. 70%, 75%, 80%, 85%, 90%, 91%, 92%, 93%, 94%, 95%, 96%, 97%, 98%, 99%, 99.5% or more) to SEQ ID NO: 5; and/or (b) comprising a fragment of at least ‘n’ consecutive amino acids of SEQ ID NO: 5, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 5. SEQ ID NO: 10 is one such fragment. Particularly preferred NadA antigens for use according to the invention comprise SEQ ID NO: 10.

fHbp (Factor H Binding Protein)

The fHbp antigen has been characterized in detail. It has also been known as protein ‘741’ (SEQ IDs 2535 & 2536 in WO99/57280), ‘NMB 1870’, ‘GNA1870’ (e.g. Masignani V. et al. (2003) J. Exp. Med. 197:789-799), ‘P2086’, ‘LP2086’ or ORF2086′ (e.g. WO03/063766). It is expressed across many meningococcal serogroups, in which it is a lipoprotein.

The fHbp antigen falls into three distinct variants (see WO2004/048404) and it has been found that for meningococci, serum raised against a given family is bactericidal within the same family but is not active against strains which express one of the other two families i.e. there is intra-family cross-protection, but not inter-family cross-protection. The invention can use a single fHbp variant, but to provide broader coverage a composition can usefully include a fHbp from two or three of the variants.

Where a composition comprises a single fHbp antigen it may include one of the following:
(a) a first polypeptide comprising a first amino acid sequence, where the first amino acid sequence comprises an amino acid sequence (i) having at least a % sequence identity to SEQ ID NO:1 (strain MC58) and/or (ii) consisting of a fragment of at least x contiguous amino acids from SEQ ID NO: 1;
(b) a second polypeptide, comprising a second amino acid sequence, where the second amino acid sequence comprises an amino acid sequence (i) having at least b % sequence identity to SEQ ID NO: 2 (strain 961-5945) and/or (ii) consisting of a fragment of at least y contiguous amino acids from SEQ ID NO: 2;
(c) a third polypeptide, comprising a third amino acid sequence, where the third amino acid sequence comprises an amino acid sequence (i) having at least c % sequence identity to SEQ ID NO: 3 (strain M1239) and/or (ii) consisting of a fragment of at least z contiguous amino acids from SEQ ID NO: 3. Where a composition comprises two different meningococcal fHbp antigens, it may include a combination of: (i) a first and second polypeptide as defined above; (ii) a first and third polypeptide as defined above; or (iii) a second and third polypeptide as defined above. A combination of a first and third polypeptide is preferred. If a single fHbp antigen is used, it is preferred that it is a first or a third polypeptide as described above.

In other embodiments a composition comprises three different meningococcal fHbp antigens, with first, second and third polypeptides as defined above. Where a composition comprises two or three different meningococcal fHbp antigens, although these may share some sequences in common, the first, second and third polypeptides have different fHbp amino acid sequences.

In some embodiments the fragment of at least x contiguous amino acids from SEQ ID NO: 1 is not also present within SEQ ID NO: 2 or within SEQ ID NO: 3. Similarly, the fragment of at least y contiguous amino acids from SEQ ID NO: 2 might not also be present within SEQ ID NO: 1 or within SEQ ID NO: 3. Similarly, the fragment of at least z contiguous amino acids from SEQ ID NO: 3 might not also be present within SEQ ID NO: 1 or within SEQ ID NO: 2. In some embodiments, when said fragment from one of SEQ ID NOS: 1 to 3 is aligned as a contiguous sequence against the other two SEQ ID NOs, the identity between the fragment and each of the other two SEQ ID NOs is less than 75% e.g. less than 70%, less than 65%), less than 60%>, etc.

The value of a is at least 80 e.g. 82, 84, 86, 88, 90, 92, 94, 95, 96, 97, 98, 99 or more. The value of b is at least 80 e.g. 82, 84, 86, 88, 90, 92, 94, 95, 96, 97, 98, 99 or more. The value of c is at least 80 e.g. 82, 84, 86, 88, 90, 92, 94, 95, 96, 97, 98, 99 or more. The values of a, b and c may be the same or different. In some embodiments, a b and c are identical.

The value of x is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 225, 250). The value of y is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 225, 250). The value of z is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 225, 250). The values of x, y and z may be the same or different. In some embodiments, x y and z are identical.

Fragments preferably comprise an epitope from the respective SEQ ID NO: sequence. Other useful fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of the respective SEQ ID NO: while retaining at least one epitope thereof.

Amino acid sequences used with the invention may, compared to SEQ ID NOs: 1, 2 or 3, include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may have one or more (e.g. 1, 2, 3, 4, 5, 6, 7, 8, 9, 10, etc.) single amino acid deletions relative to a reference sequence. The polypeptides may also include one or more (e.g. 1, 2, 3, 4, 5, 6, 7, 8, 9, 10, etc.) insertions (e.g. each of 1, 2, 3, 4 or 5 amino acids) relative to a reference sequence.

A useful first amino acid sequence has at least 85% identity (e.g. >90%, 95% or 100%) to SEQ ID NO: 1. Another useful first amino acid sequence has at least 95% identity (e.g. >98% or 100%) to SEQ ID NO: 12. Preferred fHbp sequences for use according to the invention comprise SEQ ID NO:6. A useful third amino acid sequence has at least 85% identity (e.g. >90%, 95% or 100%) to SEQ ID NO: 3. Another useful third amino acid sequence has at least 95% identity (e.g. >98% or 100%) to SEQ ID NO: 11.

Combinations comprising a mixture of first and third sequences based around SEQ ID NOs: 11 and 12 (or their close variants) are particularly useful. Thus, a composition may comprise a polypeptide comprising amino acid sequence SEQ ID NO: 11 and a further polypeptide comprising amino acid sequence SEQ ID NO: 12.

fHbp antigens used with the invention can be lipidated e.g. at a N-terminus cysteine residue. In other embodiments they will not be lipidated, and may include amino acid sequences upstream of the natural mature N-terminal cysteine. SEQ ID NOs: 1-3 and 11-12 begin with the cysteine from the natural N-terminus of the relevant mature fHbp polypeptides. For lipidated fHBPs, lipids attached to cysteines will usually include palmitoyl residues e.g. as tripalmitoyl-S-glyceryl-cysteine (Pam3Cys), dipalmitoyl-S-glyceryl cysteine (Pam2Cys), N-acetyl (dipalmitoyl-S-glyceryl cysteine), etc.

A polypeptide including the fHbp antigen sequence can include that sequence alone, or it can be a fusion polypeptide. One useful fusion partner for a fHbp sequence is the GNA2091 polypeptide, which will normally be upstream of the fHbp sequence. Thus, the fHbp antigen can be present in a composition of the invention as a GNA2091-fHbp fusion e.g. SEQ ID NO: 7.

Compositions used with the invention may also include an fHbp fusion protein comprising 2 or 3 of the first, second and third amino acid sequences defined at (a) to (c) above.

Compositions used with the invention may also include an fHbp protein that is mutated relative to SEQ ID NO: 1, 2 or 3 (fHbp variant 1, 2 or 3 respectively) to decrease binding to human factor H (fH). Suitable mutations are disclosed in Rossi et al. (2013) Vaccine 31:5451-7.

### GNA1030 Antigens

‘GNA1030’ protein from meningococcus serogroup B is disclosed as ‘953’ in WO99/57280 (SEQ IDS 2917 & 2918 therein) and as ‘NMB1030’ in Tettelin et al. (2000) Science 287:1809-1815 (see also GenBank accession number GI: 7226269). The corresponding protein in serogroup A (see Parkhill et al (2000) Nature 404:502-506) has GenBank accession number 7380108.

When used according to the present invention, GNA1030 protein may take various forms. Preferred forms of GNA1030 are truncation or deletion variants, such as those disclosed in WO01/64920, WO01/64922, and WO03/020756. In particular, the N-terminus leader peptide of GNA1030 may be deleted (i.e. deletion of residues 1 to 19 for strain MC58 [SEQ ID NO: 13]) to give GNA1030^{(NL)}.

Preferred GNA1030 sequences have 50% or more identity (e.g. 60%, 70%, 80%, 90%, 95%, 99% or more) to SEQ ID NO: 13. This includes GNA1030 variants (e.g. allelic variants, homologs, orthologs, paralogs, mutants, etc.).

Other preferred GNA1030 sequences comprise at least n consecutive amino acids from SEQ ID NO: 13, wherein nis 7 or more (eg. 8, 10, 12, 14, 16, 18, 20, 25, 30, 35, 40, 50, 60, 70, 80, 90, 100, 150, 200, 250 or more). Preferred fragments comprise an epitope from GNA1030, in which case detection of the epitope in a pathogen of interest may be performed using a monoclonal antibody to the epitope. Other preferred fragments lack one or more amino acids (e.g. 1, 2, 3, 4, 5, 6, 7, 8, 9, 10, 15, 20, 25 or more) from the C-terminus and/or the N-terminus of SEQ ID NO: 13.

### GNA2091 Antigens

‘GNA2091’ protein from meningococcus serogroup B is disclosed as protein 936 in WO99/57280 (SEQ IDs 2883 & 2884) and as ‘NMB2091’ in Tettelin et al. (2000) Science 287:1809-1815 (see also GenBank accession number GI: 7227353). The corresponding gene in serogroup A (see Parkhill et al (2000) Nature 404:502-506) has GenBank accession number 7379093.

When used according to the present invention, GNA2091 protein may take various forms. Preferred forms of GNA2091 are truncation or deletion variants, such as those disclosed in WO01/64920, WO01/64922, and WO03/020756. In particular, the N-terminus leader peptide of GNA2091 may be deleted (i.e. deletion of residues 1 to 23 for strain MC58 [SEQ ID NO: 14]) to give GNA2091 (NL).

Preferred GNA2091 sequences have 50% or more identity (e.g. 60%, 70%, 80%, 90%, 95%, 99% or more) to SEQ ID NO: 14. This includes variants (e.g. allelic variants, homologs, orthologs, paralogs, mutants etc).

Other preferred GNA2091 sequences comprise at least n consecutive amino acids from SEQ ID NO: 14, wherein n is 7 or more (eg. 8, 10, 12, 14, 16, 18, 20, 25, 30, 35, 40, 50, 60, 70, 80, 90, 100, 150, 200, 250 or more). Preferred fragments comprise an epitope from GNA2091, in which case detection of the epitope in a pathogen of interest may be performed using a monoclonal antibody to the epitope. Other preferred fragments lack one or more amino acids (e.g. 1, 2, 3, 4, 5, 6, 7, 8, 9, 10, 15, 20, 25 or more) from the C-terminus and/or the N-terminus of SEQ ID NO: 14.

Mutant fHbp Polypeptides

In another embodiment of this invention, the immunogenic composition against N. meningitidis serogroup B in liquid formulation comprises the antigens in the immunogenic composition disclosed in WO 2020/030782, which is incorporated here by reference, including the fusion polypeptides comprising mutated fHbp polypeptides. These immunogenic compositions have been found to retain the efficacy of the 4CMenB compositions (BEXSERO) but also have an improved coverage against meningococcal strains carrying fHbp variants thanks to the inclusion of the fusion fHbp polypeptides.

The lipoprotein factor H binding protein (fHbp) is expressed on the surface of all MenB strains. fHbp binds to the human complement regulatory protein factor H (hfH), forming a complex that protects the bacteria from complement-mediated killing and providing a survival mechanism for N. meningitidis in the human bloodstream. Antibodies against fHbp have a dual role: they are bactericidal per se, and by preventing binding to hfH they render strains more susceptible to bacterial killing. Reducing or abolishing the ability of fHbp to bind to hfH increases the immunogenicity of the fHbp antigen by preventing the formation of protective complexes between fHbp and hfH which have potential to mask fHbp epitopes and prevent antibody binding.

fHbp exists in three different genetic and immunogenic variants (v1, v2 and v3), with many subvariants. The majority of MenB strains that are not covered by BEXSERO express fHbp in v2, v3 or v1 subvariants distantly related to var1.1 (var1.1 being the fHbp antigen that is included in BEXSERO). WO2020/030782 discloses mutated fHbp variant 1 (v1) polypeptides that are immunogenic and can be combined with existing meningococcal vaccines to provide improved N. meningitidis strain coverage. In particular, these v1 polypeptides are subvariants of fHbp variant 1 that are genetically diverse compared with the fHbp v1.1 antigen included in BEXSERO.

Furthermore, the v1 polypeptides disclosed in WO2020/030782 are mutated in order to reduce binding to hfH compared with the corresponding wildtype v1 polypeptide. In contrast, the fHbp v1.1 antigen included in BEXSERO, and the fHp v1.55 and v3.45 antigens included in TRUMENBA, do bind to hfH.

The v1 polypeptides disclosed in WO2020/030782 can be provided alone or as a component of a fusion protein, together with mutant forms of fHbp variants 2 and 3, which have been modified to improve stability and also to reduce fHbp binding. By providing a single fusion protein comprising these v2 and v3 antigens, together with a v1 antigen of the invention, the inventors improve strain coverage. For clarity, neither of the v2 and v3 antigens are present in, e.g., BEXSERO. The presence of v2 and v3 antigens within the fusion proteins of the present invention improves strain coverage as compared to, e.g., BEXSERO.

The v1 polypeptides and fusion proteins are preferably used in combination with a meningococcal NHBA antigen, a meningococcal NadA antigen, a meningococcal fHbp antigen, and a meningococcal outer membrane vesicle (e.g., in combination with the BEXSERO composition as described above), to provide a combined immunogenic composition having increased immunogenicity (due to the addition/inclusion of non-binding forms of fHbp variants) and increased N. meningitidis serotype B strain coverage (due to the addition of new fHbp variants/subvariants), compared with BEXSERO alone.

The inventors of WO2020/030782 identified residues within the fHbp v1.13 sequence that can be modified to reduce binding to hfH. Such mutant v1.13 meningococcal fHbp polypeptides are referred to herein as non-binding (NB) mutants. The inventors also identified combinations of mutations in the v1.13 sequence that are particularly useful to reduce binding to hfH. fHbp v1.13 is also known in the art as fHbp variant B09.

The mature wild type fHbp v1.13 lipoprotein from strain M982 (GenBank Accession No. AAR84475.1) has the following amino acid sequence, with an N-terminal poly-glycine signal sequence being underlined:

(SEQ ID NO: 15)
CSSGGGGVAADIGAGLADALTAPLDHKDKGLQSLTLDQSVRKNEKLKLA
AQGAEKTYGNGDSLNTGKLKNDKVSRFDFIRQIEVDGKLITLESGEFQV
YKQSHSALTALQTEQVQDSEDSGKMVAKRQFRIGDIAGEHTSFDKLPKG
GSATYRGTAFGSDDAGGKLTYTIDFAAKQGHGKIEHLKSPELNVELATA
YIKPDEKRHAVISGSVLYNQDEKGSYSLGIFGGQAQEVAGSAEVETANG
IHHIGLAAKQ

The mature v1.13 lipoprotein differs from the full-length wild-type sequence in that the full-length polypeptide has an additional 19 residue N-terminal leader sequence, which is cleaved from the mature polypeptide. Thus, full-length wild-type fHbp v1.13 has the following amino acid sequence of SEQ ID NO: 45 (with the N-terminal leader sequence shown in bold font):

(SEQ ID NO: 45)
MNRTAFCCFSLTAALILTACSSGGGGVAADIGAGLADALTAPLDHKDKG
LQSLTLDQSVRKNEKLKLAAQGAEKTYGNGDSLNTGKLKNDKVSRFDFI
RQIEVDGKLITLESGEFQVYKQSHSALTALQTEQVQDSEDSGKMVAKRQ
FRIGDIAGEHTSFDKLPKGGSATYRGTAFGSDDAGGKLTYTIDFAAKQG
HGKIEHLKSPELNVELATAYIKPDEKRHAVISGSVLYNQDEKGSYSLGI
FGGQAQEVAGSAEVETANGIHHIGLAAKQ

The ΔG form of the mature v1.13 lipoprotein lacks the N-terminal poly-glycine sequence of the mature polypeptide, i.e. it lacks the first 7 amino acids of SEQ ID NO: 15, and it lacks the first 26 amino acids of SEQ ID NO: 45:

(SEQ ID NO: 16)
VAADIGAGLADALTAPLDHKDKGLQSLTLDQSVRKNEKLKLAAQGAEKT
YGNGDSLNTGKLKNDKVSRFDFIRQIEVDGKLITLESGEFQVYKQSHSA
LTALQTEQVQDSEDSGKMVAKRQFRIGDIAGEHTSFDKLPKGGSATYRG
TAFGSDDAGGKLTYTIDFAAKQGHGKIEHLKSPELNVELATAYIKPDEK
RHAVISGSVLYNQDEKGSYSLGIFGGQAQEVAGSAEVETANGIHHIGLA
AKQ

In one embodiment, the serogroup B antigenic component of the immunogenic composition of this invention comprises a mutant v1.13 meningococcal fHbp polypeptide comprising an amino acid sequence having at least k % sequence identity to SEQ ID NO: 16, with the proviso that the amino acid sequence of said mutant v1.13 meningococcal fHbp polypeptide includes a substitution mutation at one or more of residues E211, S216 or E232 of SEQ ID NO: 16.

The value of k may be selected from 80, 85, 86, 87, 88, 89, 90, 91, 92, 93, 94, 95, 96, 97, 98, 99 or 100. k % is preferably 80% (i.e. the mutant fHbp v1.13 amino acid sequence has at least 80% identity to SEQ ID NO: 16) and is more preferably 85%, more preferably 90% and more preferably 95%. Most preferably, the mutant fHbp v1.13 amino acid sequence has at least 97%, at least 98% or at least 99% identity to SEQ ID NO: 16.

Thus, the present invention provides a reconstituted vaccine comprising a mutant v1.13 meningococcal fHbp polypeptide comprising an amino acid sequence having at least 80% sequence identity to SEQ ID NO: 16 wherein the amino acid sequence includes a substitution mutation at one or more of residues S216, E211 or E232 of SEQ ID NO: 16.

Preferably, the amino acid sequence differs from SEQ ID NO: 16 by at least one or more of the substitutions E211A, S216R or E232A. More preferably, the amino acid sequence comprises substitutions at multiple residues selected from the following (i) E211A and E232A, or (ii) E211A and S216R. More preferably, the amino acid sequence comprises substitutions at residues E211A and S216R, relative to SEQ ID NO. 16.

Without wishing to be bound by theory, the substitution of glutamic acid (E) for alanine (A) at residue 211 of SEQ ID NO. 16 removes a negatively charged residue that is involved in hfH recruitment, thus contributing to the abrogation of fH binding. The substitution of arginine (R) for serine(S) at residue 216 of SEQ ID NO. 16 replaces the wildtype amino acid with a corresponding residue from N. gonorrhoeae, which does not bind hfH.

In preferred embodiments, a mutant v1.13 polypeptide of the invention has the amino acid sequence of SEQ ID NO: 17 (v1.13 ΔG E211A/E232A) or SEQ ID NO: 18 (v1.13 ΔG (E211A/S216R). More preferably, mutant v1.13 polypeptide of the invention has the amino acid sequence of SEQ ID NO: 18.

The mutant v1.13 polypeptide of the invention can, after administration to a host animal, preferably a mammal and more preferably a human, elicit antibodies, which can recognize wild-type meningococcal fHbp polypeptides of SEQ ID NO: 15. These antibodies are ideally bactericidal.

The inventors of WO2020/030782 also identified residues within the fHbp v1.15 sequence that can be modified to prevent binding to hfH. Such mutants are referred to herein as non-binding (NB) mutants. The inventors identified combinations of mutations in the v1.15 sequence that are particularly useful to prevent binding to hfH. fHbp v1.15 is also known in the art as fHbp variant B44.

The mature wild-type fHbp v1.15 lipoprotein from strain NM452 (GenBank Accession No. ABL14232.1) has the following amino acid sequence, with an N-terminal poly-glycine signal sequence being underlined:

(SEQ ID NO: 19)
CSSGGGGSGGGGVAADIGAGLADALTAPLDHKDKGLKSLTLEDSISQNG
TLTLSAQGAERTFKAGDKDNSLNTGKLKNDKISRFDFIRQIEVDGQLIT
LESGEFQVYKQSHSALTALQTEQVQDSEHSGKMVAKRQFRIGDIVGEHT
SFGKLPKDVMATYRGTAFGSDDAGGKLTYTIDFAAKQGHGKIEHLKSPE
LNVDLAAADIKPDEKHHAVISGSVLYNQAEKGSYSLGIFGGQAQEVAGS
AEVETANGIRHIGLAAKQ

The mature v1.15 lipoprotein differs from the full-length wild-type sequence in that the full-length polypeptide has an additional 19 residue N-terminal leader sequence, which is cleaved from the mature polypeptide. Thus, full-length wild-type fHbp v1.15 has the following amino acid sequence (with the N-terminal leader sequence shown in bold font):

(SEQ ID NO: 46)
MNRTTFCCLSLTAALILTACSSGGGGSGGGGVAADIGAGLADALTAPLD
HKDKGLKSLTLEDSISQNGTLTLSAQGAERTFKAGDKDNSLNTGKLKND
KISRFDFIRQIEVDGQLITLESGEFQVYKQSHSALTALQTEQVQDSEHS
GKMVAKRQFRIGDIVGEHTSFGKLPKDVMATYRGTAFGSDDAGGKLTYT
IDFAAKQGHGKIEHLKSPELNVDLAAADIKPDEKHHAVISGSVLYNQAE
KGSYSLGIFGGQAQEVAGSAEVETANGIRHIGLAAKQ

The ΔG form of the mature v1.15 lipoprotein lacks the N-terminal poly-glycine sequence, i.e. it lacks the first 12 amino acids of SEQ ID NO: 19, and it lacks the first 31 amino acids of SEQ ID NO: 46:

(SEQ ID NO: 20)
VAADIGAGLADALTAPLDHKDKGLKSLTLEDSISQNGTLTLSAQGAERT
FKAGDKDNSLNTGKLKNDKISRFDFIRQIEVDGQLITLESGEFQVYKQS
HSALTALQTEQVQDSEHSGKMVAKRQFRIGDIVGEHTSFGKLPKDVMAT
YRGTAFGSDDAGGKLTYTIDFAAKQGHGKIEHLKSPELNVDLAAADIKP
DEKHHAVISGSVLYNQAEKGSYSLGIFGGQAQEVAGSAEVETANGIRHI
GLAAKQ

In one embodiment, the serogroup B antigenic component of the immunogenic composition of the invention comprises an amino acid sequence having at least k % sequence identity to SEQ ID NO: 20, with the proviso that the amino acid sequence of said mutant v1.15 meningococcal fHbp polypeptide includes a substitution mutation at one or more of residues E214, S219 or E235 of SEQ ID NO: 20.

The value of k may be selected from 80, 85, 86, 87, 88, 89, 90, 91, 92, 93, 94, 95, 96, 97, 98, 99 or 100. It is preferably 80 (i.e. the mutant fHbp v1.15 amino acid sequence has at least 80% identity to SEQ ID NO: 20) and is more preferably 85, more preferably 90 and more preferably 95. Most preferably, the mutant fHbp v1.15 amino acid sequence has at least 97%, at least 98% or at least 99% identity to SEQ ID NO: 20.

Preferably, the amino acid sequence differs from SEQ ID NO: 20 by at least one or more of the substitutions E214A, S219R or E235A. More preferably, the amino acid sequence comprises substitutions at residues selected from the following: (i) S219R, (ii) E214A and S219R, and (iii) E214A and E235A.

In preferred embodiments, a mutant v1.15 polypeptide has the amino acid sequence of SEQ ID NO: 21 (v.1.15_S219R), SEQ ID NO: 22 (v1.15_E214A/S219R) or SEQ ID NO: 23 (v1.15_E214A/E235A).

The mutant v1.15 polypeptide can, after administration to a host animal, preferably a mammal and more preferably a human, elicit antibodies which can recognize wild-type meningococcal fHbp polypeptides of SEQ ID NO: 19. These antibodies are ideally bactericidal.

The disclosure in WO2020/030782 also provides a fusion polypeptide comprising all three of v1, v2 and v3 meningococcal fHbp polypeptides, wherein the variant fHbp sequences are in the order v2-v3-v1 from N- to C-terminus. In a preferred embodiment, the serogroup B antigenic component of the immunogenic composition of the invention comprises such an fHbp fusion polypeptide.

Preferably, the fHbp fusion polypeptide has an amino acid sequence of formula NH₂-A-[-X-L]₃-B—COOH, wherein each X is a different variant fHbp sequence, L is an optional linker amino acid sequence, A is an optional N terminal amino acid sequence, and B is an optional C terminal amino acid sequence.

The v1 fHbp polypeptide component of the fusion is either a mutant v1.13 fHbp polypeptide or mutant v1.13 fHbp polypeptide as described above.

The v2 and v3 fHbp polypeptide components of the fusion for inclusion in the immunogenic composition against N. meningitidis serogroup B of the invention are preferably mutant v2 and v3 polypeptides having enhanced stability and reduced ability to bind to hfH, compared to the wild type v2 and v3 polypeptides. As explained above, and without wishing to be bound by theory, reducing fHbp binding to hfH is advantageous because it prevents the formation of protective complexes between fHbp and hfH which can mask fHbp epitopes, and thereby increases the immunogenicity of the polypeptide antigens.

Residues within the v2 and v3 sequences, which can be modified to increase the stability of the polypeptide and also to reduce binding to hfH, have been identified and described in detail in WO2015/128480.

Full-length wild-type fHbp v2 from strain 2996 has the following amino acid sequence (leader sequence shown in bold font and poly-glycine sequence being underlined):

(SEQ ID NO: 24)
MNRTAFCCLSLTAALILTACSSGGGGVAADIGAGLADALTAPLDHKDKS
LQSLTLDQSVRKNEKLKLAAQGAEKTYGNGDSLNTGKLKNDKVSRFDFI
RQIEVDGQLITLESGEFQIYKQDHSAVVALQIEKINNPDKIDSLINQRS
FLVSGLGGEHTAFNQLPDGKAEYHGKAFSSDDAGGKLTYTIDFAAKQGH
GKIEHLKTPEQNVELAAAELKADEKSHAVILGDTRYGSEEKGTYHLALF
GDRAQEIAGSATVKIGEKVHEIGIAGKQ

The mature lipoprotein lacks the first 19 amino acids of SEQ ID NO: 24:

(SEQ ID NO: 25)
CSSGGGGVAADIGAGLADALTAPLDHKDKSLQSLTLDQSVRKNEKLKLA
AQGAEKTYGNGDSLNTGKLKNDKVSRFDFIRQIEVDGQLITLESGEFQI
YKQDHSAVVALQIEKINNPDKIDSLINQRSFLVSGLGGEHTAFNQLPDG
KAEYHGKAFSSDDAGGKLTYTIDFAAKQGHGKIEHLKTPEQNVELAAAE
LKADEKSHAVILGDTRYGSEEKGTYHLALFGDRAQEIAGSATVKIGEKV
HEIGIAGKQ

The ΔG form of SEQ ID NO: 24 lacks the first 26 amino acids:

(SEQ ID NO: 26)
VAADIGAGLADALTAPLDHKDKSLQSLTLDQSVRKNEKLKLAAQGAEKT
YGNGDSLNTGKLKNDKVSRFDFIRQIEVDGQLITLESGEFQIYKQDHSA
VVALQIEKINNPDKIDSLINQRSFLVSGLGGEHTAFNQLPDGKAEYHGK
AFSSDDAGGKLTYTIDFAAKQGHGKIEHLKTPEQNVELAAAELKADEKS
HAVILGDTRYGSEEKGTYHLALFGDRAQEIAGSATVKIGEKVHEIGIAG
KQ

In a preferred embodiment, the fusion polypeptide of the invention comprises a mutant v2 fHbp polypeptide comprising an amino acid sequence having at least k % sequence identity to SEQ ID NO: 26, with the proviso that the v2 fHbp amino acid sequence includes a substitution mutation at residues S32 and L123 of SEQ ID NO: 26. Preferably the substitutions are S32V and L123R.

The value of k may be selected from 80, 85, 86, 87, 88, 89, 90, 91, 92, 93, 94, 95, 96, 97, 98, 99 or 100. k % is preferably 80% (i.e. the mutant fHbp v2 amino acid sequence has at least 80% identity to SEQ ID NO: 26) and is more preferably 85%, more preferably 90% and more preferably 95%.

In some embodiments, the fHbp v2 polypeptide included in the fusion protein of the invention is truncated relative to SEQ ID NO: 26. Compared to the wild-type mature sequence, SEQ ID NO: 26 is already truncated at the N-terminus up to and including the poly-glycine sequence (compare SEQ ID NOs: 25 and 26), but SEQ ID NO: 26 can be truncated at the C-terminus and/or further truncated at the N-terminus.

In a preferred embodiment, the v2 fHbp polypeptide included in the fusion protein of the invention comprises or consists of the amino acid sequence of SEQ ID NO: 30. The v2 fHbp polypeptide included in the fusion protein has, under the same experimental conditions, a higher stability than the same polypeptide but without the sequence differences at residues S32 and L123 e.g. higher stability than a wild-type meningococcal polypeptide consisting of SEQ ID NO: 24. The S32V mutation stabilizes the structure by introducing favorable hydrophobic interactions. Without wishing to be bound to theory, the L123R mutation abrogates fH binding by introducing clashes with fH and unfavorable charges.

The stability enhancement can be assessed using differential scanning calorimetry (DSC) e.g. as discussed in Johnson (2013) Arch Biochem Biophys 531:100-9 and Bruylants et al. Current Medicinal Chemistry 2005; 12:2011-20. DSC has previously been used to assess the stability of v2 fHbp (Johnson et al. PLoS Pathogen 2012; 8: e1002981). Suitable conditions for DSC to assess stability can use 20 pM of polypeptide in a buffered solution (e.g. 25 mM Tris) with a pH between 6 and 8 (e.g. 7-7.5) with 100-200 mM NaCl (e.g. 150 mM).

The increase in stability is evidenced by an at least 5° C., e.g. at least 10° C., 15° C., 20° C., 25° C., 30° C., 35° C. or more, increase in thermal transition midpoint (Tm) of at least one peak as compared to wildtype when assessed by DSC. Wild-type fHbp shows two DSC peaks during unfolding (one for the N-terminal domain and one for the C-terminal domain) and, where a v2 polypeptide included in the fusion protein of the invention includes both such domains, an “increase in stability” refers to an at least 5° C. increase in the Tₘ of the N-terminal domain. Tm of the N-terminal domain can occur at or even below 40° C. with wild-type v2 sequences (Johnson et al. (2012) PLoS Pathogen 8: e1002981), whereas C-terminal domains can have a Tm of 80° C. or more. Thus, the mutant fHbp v2 amino acid sequence included in the fusion protein of the invention preferably has a N-terminal domain with a Tm of at least 45° C. e.g. >50° C., >55° C., >60° C., >65° C., >70° C., >75° C., or even >80° C.

Full-length wild-type fHbp v3 from strain M1239 has the following amino acid sequence (leader sequence shown in bold font and poly-glycine sequence being underlined):

(SEQ ID NO: 27)
MNRTAFCCLSLTTALILTACSSGGGGSGGGGVAADIGTGLADALTAPLD
HKDKGLKSLTLEDSIPQNGTLTLSAQGAEKTFKAGDKDNSLNTGKLKND
KISRFDFVQKIEVDGQTITLASGEFQIYKQNHSAVVALQIEKINNPDKT
DSLINQRSFLVSGLGGEHTAFNQLPGGKAEYHGKAFSSDDPNGRLHYSI
DFTKKQGYGRIEHLKTLEQNVELAAAELKADEKSHAVILGDTRYGSEEK
GTYHLALFGDRAQEIAGSATVKIGEKVHEIGIAGKQ

The mature lipoprotein lacks the first 19 amino acids of SEQ ID NO: 27:

(SEQ ID NO: 28)
CSSGGGGSGGGGVAADIGTGLADALTAPLDHKDKGLKSLTLEDSIPQNG
TLTLSAQGAEKTFKAGDKDNSLNTGKLKNDKISRFDFVQKIEVDGQTIT
LASGEFQIYKQNHSAVVALQIEKINNPDKTDSLINQRSFLVSGLGGEHT
AFNQLPGGKAEYHGKAFSSDDPNGRLHYSIDFTKKQGYGRIEHLKTLEQ
NVELAAAELKADEKSHAVILGDTRYGSEEKGTYHLALFGDRAQEIAGSA
TVKIGEKVHEIGIAGKQ

The ΔG form of SEQ ID NO: 27 lacks the first 31 amino acids (i.e. lacks the signal sequence and the poly-glycine sequence):

(SEQ ID NO: 29)
VAADIGTGLADALTAPLDHKDKGLKSLTLEDSIPQNGTLTLSAQGAEKT
FKAGDKDNSLNTGKLKNDKISRFDFVQKIEVDGQTITLASGEFQIYKQN
HSAVVALQIEKINNPDKTDSLINQRSFLVSGLGGEHTAFNQLPGGKAEY
HGKAFSSDDPNGRLHYSIDFTKKQGYGRIEHLKTLEQNVELAAAELKAD
EKSHAVILGDTRYGSEEKGTYHLALFGDRAQEIAGSATVKIGEKVHEIG
IAGKQ

In a preferred embodiment, the fusion polypeptide of the invention comprises a mutant v3 fHbp polypeptide comprising an amino acid sequence having at least k % sequence identity to SEQ ID NO: 29, with the proviso that the v3 fHbp amino acid sequence includes substitution mutations at residues S32 and L126 of SEQ ID NO: 29. Preferably the substitutions are S32V and L126R.

The value of k may be selected from 80, 85, 86, 87, 88, 89, 90, 91, 92, 93, 94, 95, 96, 97, 98, 99 or 100. k % is preferably 80% (i.e. the mutant fHbp v2 amino acid sequence has at least 80% identity to SEQ ID NO: 29) and is more preferably 85%, more preferably 90% and more preferably 95%.

In some embodiments, the fHbp v3 polypeptide included in the fusion protein of the invention is truncated relative to SEQ ID NO: 29. Compared to the wild-type mature sequence, SEQ ID NO: 29 is already truncated at the N-terminus up to and including the poly-glycine sequence (compare SEQ ID NOs: 28 and 29), but SEQ ID NO: 29 can be truncated at the C-terminus and/or further truncated at the N-terminus.

In a preferred embodiment, the v3 fHbp polypeptide included in the fusion protein of the invention comprises or consists of the amino acid sequence of SEQ ID NO: 31. The v3 fHbp polypeptide included in the fusion protein has, under the same experimental conditions, a higher stability than the same polypeptide but without the sequence differences at residues S32 and L126 e.g. higher stability than a wild-type meningococcal polypeptide consisting of SEQ ID NO: 27. The S32V mutation stabilizes the structure by introducing favorable hydrophobic interactions. Without wishing to be bound by theory, the L126R mutation abrogates fH binding by introducing clashes with fH and unfavorable charges.

The stability enhancement can be assessed using differential scanning calorimetry (DSC) e.g. as discussed in Johnson (2013) Arch Biochem Biophys 531:100-9 and Bruylants et al. (2005) Current Medicinal Chemistry 12:2011-20. DSC has previously been used to assess the stability of v3 fHbp (van der Veen et al. (2014) Infect Immun PMID 24379280). Suitable conditions for DSC to assess stability can use 20 μM of polypeptide in a buffered solution (e.g. 25 mM Tris) with a pH between 6 and 8 (e.g. 7-7.5) with 100-200 mM NaCl (e.g. 150 mM).

The increase in stability is evidenced by an at least 5° C., e.g. at least 10° C., 15° C., 20° C., 25° C., 30° C., 35° C. or more, increase in thermal transition midpoint (Tm) of at least one peak as compared to wildtype when assessed by DSC. Wild-type fHbp shows two DSC peaks during unfolding (one for the N-terminal domain and one for the C-terminal domain) and, where a v3 polypeptide included in the fusion protein of the invention includes both such domains, an “increase in stability” refers to an at least 5° C. increase in the Tm of the N-terminal domain. Tm of the N terminal domain can occur at around 60° C. or less with wild-type v3 sequences (Johnson et al. (2012) PLoS Pathogen 8:e1002981), whereas C-terminal domains can have a Tm of 80° C. or more. Thus, the mutant fHbp v3 amino acid sequence of the invention preferably has a N-terminal domain with a Tm of at least 65° C. e.g. >70° C., >75° C., or even >80° C.

As described above, in a preferred embodiment the fHbp fusion polypeptide has an amino acid sequence of formula NH₂-A-[-X-L]₃-B—COOH, wherein each X is a different variant fHbp sequence and L is an optional linker amino acid sequence. In a preferred embodiment, the linker amino acid sequence “L” is a glycine polymer or glycine-serine polymer linker.

Exemplary linkers include, but are not limited to, GGSG (SEQ ID NO:50), GGSGG (SEQ ID NO:51), GSGSG (SEQ ID NO:52), GSGGG (SEQ ID NO:53), GGGSG (SEQ ID NO:54), GSSSG (SEQ ID NO:55) and GSGGGG (SEQ ID NO:56). Other suitable glycine or glycine-serine polymer linkers will be apparent to the skilled person. In a preferred fusion polypeptide according to the invention, the v2 and v3 sequences and the v3 and v1 sequences are connected by the glycine-serine polymer linker GSGGGG (SEQ ID NO:56).

In a preferred embodiment, the fusion polypeptide of the invention comprises or consists of one of the following amino acid sequences (glycine-serine linker sequences are underlined and mutated residues are indicated in bold font):

fHbp 23S_1.13_E211A/E232A
(SEQ ID NO: 32)
VAADIGAGLADALTAPLDHKDKSLQSLTLDQVVRKNEKLKLAAQGAEKTYGNGDSLNTGKLKNDKVSRFDFIRQ
IEVDGQLITLESGEFQIYKQDHSAVVALQIEKINNPDKIDSLINQRSFRVSGLGGEHTAFNQLPDGKAEYHGKAFS
SDDAGGKLTYTIDFAAKQGHGKIEHLKTPEQNVELAAAELKADEKSHAVILGDTRYGSEEKGTYHLALFGDRAQE
IAGSATVKIGEKVHEIGIAGKQGSGGGGVAADIGTGLADALTAPLDHKDKGLKSLTLEDVIPQNGTLTLSAQGA
EKTFKAGDKDNSLNTGKLKNDKISRFDFVQKIEVDGQTITLASGEFQIYKQNHSAVVALQIEKINNPDKTDSLIN
QRSFRVSGLGGEHTAFNQLPGGKAEYHGKAFSSDDPNGRLHYSIDFTKKQGYGRIEHLKTLEQNVELAAAELKA
DEKSHAVILGDTRYGSEEKGTYHLALFGDRAQEIAGSATVKIGEKVHEIGIAGKQGSGGGGVAADIGAGLADAL
TAPLDHKDKGLQSLTLDQSVRKNEKLKLAAQGAEKTYGNGDSLNTGKLKNDKVSRFDFIRQIEVDGKLITLESGE
FQVYKQSHSALTALQTEQVQDSEDSGKMVAKRQFRIGDIAGEHTSFDKLPKGGSATYRGTAFGSDDAGGKLTY
TIDFAAKQGHGKIEHLKSPELNVELATAYIKPDEKRHAVISGSVLYNQDAKGSYSLGIFGGQAQEVAGSAAVETA
NGIHHIGLAAKQ
fHbp 23S_1.13_E211A/S216R
(SEQ ID NO: 33)
VAADIGAGLADALTAPLDHKDKSLQSLTLDQVVRKNEKLKLAAQGAEKTYGNGDSLNTGKLKNDKVSRFDFIRQ
IEVDGQLITLESGEFQIYKQDHSAVVALQIEKINNPDKIDSLINQRSFRVSGLGGEHTAFNQLPDGKAEYHGKAFS
SDDAGGKLTYTIDFAAKQGHGKIEHLKTPEQNVELAAAELKADEKSHAVILGDTRYGSEEKGTYHLALFGDRAQE
IAGSATVKIGEKVHEIGIAGKQGSGGGGVAADIGTGLADALTAPLDHKDKGLKSLTLEDVIPQNGTLTLSAQGA
EKTFKAGDKDNSLNTGKLKNDKISRFDFVQKIEVDGQTITLASGEFQIYKQNHSAVVALQIEKINNPDKTDSLIN
QRSFRVSGLGGEHTAFNQLPGGKAEYHGKAFSSDDPNGRLHYSIDFTKKQGYGRIEHLKTLEQNVELAAAELKA
DEKSHAVILGDTRYGSEEKGTYHLALFGDRAQEIAGSATVKIGEKVHEIGIAGKQGSGGGGVAADIGAGLADAL
TAPLDHKDKGLQSLTLDQSVRKNEKLKLAAQGAEKTYGNGDSLNTGKLKNDKVSRFDFIRQIEVDGKLITLESGE
FQVYKQSHSALTALQTEQVQDSEDSGKMVAKRQFRIGDIAGEHTSFDKLPKGGSATYRGTAFGSDDAGGKLTY
TIDFAAKQGHGKIEHLKSPELNVELATAYIKPDEKRHAVISGSVLYNQDAKGSYRLGIFGGQAQEVAGSAEVETA
NGIHHIGLAAKQ
fHbp_23S_1.15_S219R
(SEQ ID NO: 34)
VAADIGAGLADALTAPLDHKDKSLQSLTLDQVVRKNEKLKLAAQGAEKTYGNGDSLNTGKLKNDKVSRFDFIRQ
IEVDGQLITLESGEFQIYKQDHSAVVALQIEKINNPDKIDSLINQRSFRVSGLGGEHTAFNQLPDGKAEYHGKAFS
SDDAGGKLTYTIDFAAKQGHGKIEHLKTPEQNVELAAAELKADEKSHAVILGDTRYGSEEKGTYHLALFGDRAQE
IAGSATVKIGEKVHEIGIAGKQGSGGGGVAADIGTGLADALTAPLDHKDKGLKSLTLEDVIPQNGTLTLSAQGA
EKTFKAGDKDNSLNTGKLKNDKISRFDFVQKIEVDGQTITLASGEFQIYKQNHSAVVALQIEKINNPDKTDSLIN
QRSFRVSGLGGEHTAFNQLPGGKAEYHGKAFSSDDPNGRLHYSIDFTKKQGYGRIEHLKTLEQNVELAAAELKA
DEKSHAVILGDTRYGSEEKGTYHLALFGDRAQEIAGSATVKIGEKVHEIGIAGKQGSGGGGVAADIGAGLADAL
TAPLDHKDKGLKSLTLEDSISQNGTLTLSAQGAERTFKAGDKDNSLNTGKLKNDKISRFDFIRQIEVDGQLITLES
GEFQVYKQSHSALTALQTEQVQDSEHSGKMVAKRQFRIGDIVGEHTSFGKLPKDVMATYRGTAFGSDDAGGKL
TYTIDFAAKQGHGKIEHLKSPELNVDLAAADIKPDEKHHAVISGSVLYNQAEKGSYRLGIFGGQAQEVAGSAEVE
TANGIRHIGLAAKQ
fHbp_23S_1.15_E214A/S219R
(SEQ ID NO: 35)
VAADIGAGLADALTAPLDHKDKSLQSLTLDQVVRKNEKLKLAAQGAEKTYGNGDSLNTGKLKNDKVSRFDFIRQ
IEVDGQLITLESGEFQIYKQDHSAVVALQIEKINNPDKIDSLINQRSFRVSGLGGEHTAFNQLPDGKAEYHGKAFS
SDDAGGKLTYTIDFAAKQGHGKIEHLKTPEQNVELAAAELKADEKSHAVILGDTRYGSEEKGTYHLALFGDRAQE
IAGSATVKIGEKVHEIGIAGKQGSGGGGVAADIGTGLADALTAPLDHKDKGLKSLTLEDVIPQNGTLTLSAQGA
EKTFKAGDKDNSLNTGKLKNDKISRFDFVQKIEVDGQTITLASGEFQIYKQNHSAVVALQIEKINNPDKTDSLIN
QRSFRVSGLGGEHTAFNQLPGGKAEYHGKAFSSDDPNGRLHYSIDFTKKQGYGRIEHLKTLEQNVELAAAELKA
DEKSHAVILGDTRYGSEEKGTYHLALFGDRAQEIAGSATVKIGEKVHEIGIAGKQGSGGGGVAADIGAGLADAL
TAPLDHKDKGLKSLTLEDSISQNGTLTLSAQGAERTFKAGDKDNSLNTGKLKNDKISRFDFIRQIEVDGQLITLES
GEFQVYKQSHSALTALQTEQVQDSEHSGKMVAKRQFRIGDIVGEHTSFGKLPKDVMATYRGTAFGSDDAGGKL
TYTIDFAAKQGHGKIEHLKSPELNVDLAAADIKPDEKHHAVISGSVLYNQAAKGSYRLGIFGGQAQEVAGSAEVE
TANGIRHIGLAAKQ
fHbp_23S_1.15_E214A/E235A
(SEQ ID NO: 36)
VAADIGAGLADALTAPLDHKDKSLQSLTLDQVVRKNEKLKLAAQGAEKTYGNGDSLNTGKLKNDKVSRFDFIRQ
IEVDGQLITLESGEFQIYKQDHSAVVALQIEKINNPDKIDSLINQRSFRVSGLGGEHTAFNQLPDGKAEYHGKAFS
SDDAGGKLTYTIDFAAKQGHGKIEHLKTPEQNVELAAAELKADEKSHAVILGDTRYGSEEKGTYHLALFGDRAQE
IAGSATVKIGEKVHEIGIAGKQGSGGGGVAADIGTGLADALTAPLDHKDKGLKSLTLEDVIPQNGTLTLSAQGA
EKTFKAGDKDNSLNTGKLKNDKISRFDFVQKIEVDGQTITLASGEFQIYKQNHSAVVALQIEKINNPDKTDSLIN
QRSFRVSGLGGEHTAFNQLPGGKAEYHGKAFSSDDPNGRLHYSIDFTKKQGYGRIEHLKTLEQNVELAAAELKA
DEKSHAVILGDTRYGSEEKGTYHLALFGDRAQEIAGSATVKIGEKVHEIGIAGKQGSGGGGVAADIGAGLADAL
TAPLDHKDKGLKSLTLEDSISQNGTLTLSAQGAERTFKAGDKDNSLNTGKLKNDKISRFDFIRQIEVDGQLITLES
GEFQVYKQSHSALTALQTEQVQDSEHSGKMVAKRQFRIGDIVGEHTSFGKLPKDVMATYRGTAFGSDDAGGKL
TYTIDFAAKQGHGKIEHLKSPELNVDLAAADIKPDEKHHAVISGSVLYNQAAKGSYSLGIFGGQAQEVAGSAAVE
TANGIRHIGLAAKQ

In a preferred embodiment, the fusion polypeptide of the invention comprises the amino acid sequence of SEQ ID NO. 33. In an alternative preferred embodiment, the fusion polypeptide of the invention comprises the amino acid sequence of SEQ ID NO. 32.

The fusion polypeptide of the invention can, after administration to a host animal, preferably a mammal and more preferably a human, elicit antibodies which can recognise wild-type meningococcal fHbp polypeptides, in particular the polypeptides of SEQ ID NO: 45, 46, 24 and/or 27. These antibodies are ideally bactericidal.

As described above, in a preferred embodiment an fHbp fusion polypeptide according to the invention has an amino acid sequence of formula NH₂-A-[-X-L]₃-B—COOH, wherein each X is a different variant fHbp sequence and A is an optional N terminal amino acid sequence. In preferred embodiments, fusion proteins described herein further comprise the following N-terminal amino acid sequence, which is advantageous for enabling good expression of the fusion protein:

(SEQ ID NO. 48)
MGPDSDRLQQRR

Any of the fusion proteins disclosed herein (e.g. SEQ ID Nos. 32-36, 43 and 44) may be modified to include the amino acid sequence of SEQ ID NO. 48 at the N-terminal of the fusion polypeptide, i.e.

the amino acid sequence of SEQ ID NO. 48 is added to the N-terminal of the fHbp v2 component of the fusion polypeptide.

In a preferred embodiment, the serogroup B antigenic component of the immunogenic composition of the invention comprises the complete BEXSERO vaccine product, together with an fHbp fusion polypeptide as defined above. Most preferably, the fHbp fusion polypeptide is fHbp 23S_1.13_E211A/S216R. Preferably, the serogroup B antigenic component is provided in a single fully liquid formulation.

Preferred v1.13, v1.15 and/or fusion polypeptides described above can elicit antibody responses that are bactericidal against meningococci. Bactericidal antibody responses are conveniently measured in mice and are a standard indicator of vaccine efficacy (e.g. see end-note 14 of Pizza et al. (2000) Science 287:1816-1820; also WO2007/028408).

Polypeptides described above can preferably elicit an antibody response which is bactericidal against a N. meningitidis serogroup B strain which expresses a v1.13 fHbp sequence.

Preferred polypeptides described above can elicit antibodies in a mouse which are bactericidal against a N. meningitidis strain which expresses a v1.13 fHbp sequence in a serum bactericidal assay.

Polypeptides described above can preferably elicit an antibody response which is bactericidal against a N. meningitidis serogroup B strain which expresses a v1.15 fHbp sequence.

Preferred polypeptides described above can elicit antibodies in a mouse which are bactericidal against a N. meningitidis strain which expresses a v1.15 fHbp sequence in a serum bactericidal assay.

For example, an immunogenic composition comprising these polypeptides can provide a serum bactericidal titer of >1:4 using the Goldschneider assay with human complement [Goldschneider et al. (1969) J. Exp. Med. 129:1307-26, Santos et al. (2001) Clinical and Diagnostic Laboratory Immunology 8:616-23, and Frasch et al. (2009) Vaccine 27S:B112-6], and/or providing a serum bactericidal titer of >1:128 using baby rabbit complement.

Polypeptides described above can be prepared by various means e.g. by chemical synthesis (at least in part), by digesting longer polypeptides using proteases, by translation from RNA, by purification from cell culture (e.g. from recombinant expression or from N. meningitidis culture), etc.

Heterologous expression in an E. coli host is a preferred expression route.

Polypeptides are ideally at least 100 amino acids long e.g. 150 aa, 175 aa, 200 aa, 225 aa, or longer. They include a mutant fHbp v1, v2 and/or v3 amino acid sequence, and the mutant fHbp v1, v2 or v3 amino acid sequence should similarly be at least 100 amino acids long e.g. 150 aa, 175 aa, 200 aa, 225 aa, or longer.

The fHbp is naturally a lipoprotein in N. meningitidis. It has also been found to be lipidated when expressed in E. coli with its native leader sequence or with heterologous leader sequences. Polypeptides of the invention may have an N-terminus cysteine residue, which may be lipidated e.g. comprising a palmitoyl group, usually forming tripalmitoyl-S-glyceryl-cysteine. In other embodiments the polypeptides are not lipidated.

Polypeptides are preferably prepared in substantially pure or substantially isolated form (i.e. substantially free from other Neisserial or host cell polypeptides). In general, the polypeptides are provided in a non-naturally occurring environment e.g. they are separated from their naturally-occurring environment. In certain embodiments, the polypeptide is present in a composition that is enriched for the polypeptide as compared to a starting material. Thus, purified polypeptide is provided, whereby purified means that the polypeptide is present in a composition that is substantially free of other expressed polypeptides, whereby substantially free is meant that more than 50% (e.g. >75%, >80%, >90%, >95%, or >99%) of total polypeptide in the composition is a polypeptide of the invention.

Polypeptides can take various forms (e.g. native, fusions, glycosylated, non-glycosylated, lipidated, disulfide bridges, etc.).

If a polypeptide is produced by translation in a biological host then a start codon is required, which will provide a N-terminus methionine in most hosts. Thus, a polypeptide will, at least at a nascent stage, include a methionine residue upstream of said SEQ ID NO sequence.

Cleavage of nascent sequences means that the mutant fHbp v1, v2 or v3 amino acid sequence might itself provide the polypeptide N-terminus. In other embodiments, however, a polypeptide can include a N-terminal sequence upstream of the mutant fHbp v1, v2 or v3 amino acid sequence. In some embodiments the polypeptide has a single methionine at the N-terminus immediately followed by the mutant fHbp v1, v2 or v3 amino acid sequence; in other embodiments a longer upstream sequence may be used. Such an upstream sequence may be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. a histidine tag i.e. Hisₙ where n is 4, 5, 6, 7, 8, 9, 10 or more). Other suitable N-terminal amino acid sequences will be apparent to those skilled in the art.

A polypeptide may also include amino acids downstream of the final amino acid of the mutant fHbp v1, v2 or v3 amino acid sequence. Such C-terminal extensions may be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising a histidine tag i.e. Hisₙ where n=4, 5, 6, 7, 8, 9, 10 or more), or sequences which enhance polypeptide stability. Other suitable C-terminal amino acid sequences will be apparent to those skilled in the art.

In some embodiments, the invention excludes polypeptides which include a histidine tag (cf. Johnson et al. (2012) PLoS Pathogen 8:e1002981, and Pajon et al. (2012) Infect Immun 80:2667-77), and in particular a hexahistidine tag at the C-terminus.

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains.

Polypeptides may be attached or immobilized to a solid support.

Polypeptides may comprise a detectable label e.g. a radioactive label, a fluorescent label, or a biotin label. This is particularly useful in immunoassay techniques.

Polypeptides typically consist of an artificial amino acid sequence, namely a sequence which is not present in any naturally-occurring meningococci.

Affinity for factor H can be quantitatively assessed using surface plasmon resonance (e.g. as disclosed in Schneider et al. (2009) Nature 458:890-5) with immobilized human fH. Mutations which provide an affinity reduction (i.e. an increase in the dissociation constant, KD) of at least 10-fold, and ideally at least 100-fold, is preferred (when measured under the same experimental conditions relative to the same polypeptide but without the mutation).

### Further Antigenic Components

Immunogenic compositions of this invention may include antigens for immunizing against other diseases or infections. For example, the composition may include one or more of the following further antigens:
a saccharide antigen from Streptococcus pneumoniae [e.g. Watson (2000) Pediatr Infect Dis J 19: 331-332, Rubin (2000) Pediatr Clin North Am 47:269-285, and Jedrzejas (2001) Microbiol Mol Biol Rev 65:187-207].
an antigen from hepatitis A virus, such as inactivated virus [e.g. Bell (2000) Pediatr Infect Dis J 19:1187-1188, Iwarson (1995) APMIS 103:321-326].
an antigen from hepatitis B virus, such as the surface and/or core antigens [e.g. Gerlich et al. (1990) Vaccine 8 Suppl: S63-68 & 79-80].
a diphtheria antigen, such as a diphtheria toxoid [e.g. chapter 3 of Vaccines (1988) eds. Plotkin & Mortimer. ISBN 0-7216-1946-0] e.g. the CRM₁₉₇ mutant [e.g. Del Giudice et al. (1998) Molecular Aspects of Medicine 19:1-70].
a tetanus antigen, such as a tetanus toxoid (e.g. chapter 4 of Vaccines (1988) eds. Plotkin & Mortimer. ISBN 0-7216-1946-0).
an antigen from Bordetella pertussis, such as pertussis holotoxin (PT) and filamentous haemagglutinin (FHA) from B. pertussis, optionally also in combination with pertactin and/or agglutinogens 2 and 3 (e.g. Gustafsson et al. (1996) N. Engl. J. Med. 334:349-355, and Rappuoli et al. (1991) TIBTECH 9: 232-238).
a saccharide antigen from Haemophilus influenzae B [e.g. Costantino et al. (1999) Vaccine 17:1251-1263].
polio antigen(s) [e.g. Sutter et al. (2000) Pediatr Clin North Am 47:287-308, and Zimmerman & Spann (1999) Am Fam Physician 59:113-118, 125-126], such as IPV.
measles, mumps and/or rubella antigens (e.g. chapters 9, 10 & 11 of Vaccines (1988) eds. Plotkin & Mortimer. ISBN 0-7216-1946-0).
influenza antigen(s) (e.g. chapter 19 of Vaccines (1988) eds. Plotkin & Mortimer. ISBN 0-7216-1946-0), such as the haemagglutinin and/or neuraminidase surface proteins.
an antigen from Moraxella catarrhalis [e.g. McMichael (2000) Vaccine 19 Suppl 1: S101-107].
a protein antigen from Streptococcus agalactiae (group B streptococcus) [e.g. Schuchat (1999) Lancet 353 (9146): 51-6, WO02/34771].
a saccharide antigen from Streptococcus agalactiae (group B streptococcus).
an antigen from Streptococcus pyogenes (group A streptococcus) [e.g. WO02/34771, Dale (1999) Infect Dis Clin North Am 13:227-43, Ferretti et al. (2001) PNAS USA 98:4658-4663].
an antigen from Staphylococcus aureus [e.g. Kuroda et al. (2001) Lancet 357 (9264): 1225-1240; see also pages 1218-1219].

In an embodiment the immunogenic compositions of the present invention do not comprise an antigen against N. meningitidis serogroup X.

Toxic protein antigens may be detoxified where necessary (e.g. detoxification of pertussis toxin by chemical and/or genetic means [Rappuoli et al. (1991) TIBTECH9: 232-238]).

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens. DTP combinations are thus preferred.

Saccharide antigens are preferably in the form of conjugates. In general, conjugation enhances the immunogenicity of saccharides as it converts them from T-independent antigens to T-dependent antigens, thus allowing priming for immunological memory. Conjugation is particularly useful for paediatric vaccines and is a well-known technique.

Typical carrier proteins are bacterial toxins, such as diphtheria or tetanus toxins, or toxoids or mutants thereof. The CRM₁₉₇ diphtheria toxin mutant [Research Disclosure, 453077 (January 2002)] is useful, and is the carrier in the Streptococcus pneumoniae vaccine sold under the trade name PREVNAR. Also useful as carrier protein is recombinant CRM₁₉₇ (rCRM₁₉₇) obtained or derived from Pseudomonas fluorescens or from Escherichia coli. Other suitable carrier proteins include the N. meningitidis outer membrane protein complex [EP-A-0372501], synthetic peptides [EP-A-0378881, EP-A-0427347], heat shock proteins [WO93/17712, WO94/03208], pertussis proteins [WO98/58668, EP-A-0471177], cytokines [WO91/01146], lymphokines [WO91/01146], hormones [WO91/01146], growth factors [WO91/01146], artificial proteins comprising multiple human CD4⁺ T cell epitopes from various pathogen-derived antigens [Falugi et al. (2001) Eur J Immunol 31:3816-3824] such as N19 [Baraldo et al. (2004) Infect Immun 72(8):4884-7], protein D from H. influenzae [EP-A-0594610, Ruan et al. (1990) J Immunol 145:3379-3384] pneumolysin [Kuo et al. (1995) Infect Immun 63:2706-13] or its non-toxic derivatives [Michon et al. (1998) Vaccine. 16:1732-41], pneumococcal surface protein PspA [WO02/091998], iron-uptake proteins [WO01/72337], toxin A or B from C. difficile [WO00/61761], recombinant P. aeruginosa exoprotein A (rEPA) [WO00/33882], etc.

Any suitable conjugation reaction can be used, with any suitable linker where necessary.

The saccharide will typically be activated or functionalized prior to conjugation. Activation may involve, for example, cyanylating reagents such as CDAP (e.g. 1-cyano-4-dimethylamino pyridinium tetrafluoroborate [Lees et al. (1996) Vaccine 14:190-198, WO95/08348]). Other suitable techniques use carbodiimides, hydrazides, active esters, norbornane, p-nitrobenzoic acid, N-hydroxysuccinimide, S-NHS, EDC, TSTU, etc.

Linkages via a linker group may be made using any known procedure, for example, the procedures described in U.S. Pat. Nos. 4,882,317 and 4,695,624. One type of linkage involves reductive amination of the polysaccharide, coupling the resulting amino group with one end of an adipic acid linker group, and then coupling a protein to the other end of the adipic acid linker group [Porro et al. (1985) Mol Immunol 22:907-919, EP0208375]. Other linkers include B-propionamido [WO00/10599], nitrophenyl-ethylamine [Gever et al. Med. Microbiol. Immunol, 165:171-288 (1979)], haloacyl halides [U.S. Pat. No. 4,057,685], glycosidic linkages [U.S. Pat. Nos. 4,673,574; 4,761,283; 4,808,700], 6-aminocaproic acid [U.S. Pat. No. 4,459,286], ADH [U.S. Pat. No. 4,965,338], C4 to C12 moieties [U.S. Pat. No. 4,663,160] etc. As an alternative to using a linker, direct linkage can be used. Direct linkages to the protein may comprise oxidation of the polysaccharide followed by reductive amination with the protein, as described in, for example, U.S. Pat. Nos. 4,761,283 and 4,356,170.

A process involving the introduction of amino groups into the saccharide (e.g. by replacing terminal ═O groups with —NH₂) followed by derivatization with an adipic diester (e.g. adipic acid N-hydroxysuccinimido diester) and reaction with carrier protein is preferred. Another preferred reaction uses CDAP activation with a protein D carrier e.g. for MenA or MenC.

Antigens in the composition will typically be present at a concentration of at least 1 μg/ml each. In general, the concentration of any given antigen will be sufficient to elicit an immune response against that antigen.

Immunogenic compositions of the invention may be used therapeutically (i.e. to treat an existing infection) or prophylactically (i.e. to prevent future infection).

As an alternative to using protein antigens in the immunogenic compositions of the invention, nucleic acid (which could be RNA, such as a self-replicating RNA, or DNA, such as a plasmid) encoding the antigen may be used.

### Non-Antigenic Components and Formulation of the Compositions

The immunogenic compositions of the invention will generally include one or more pharmaceutically acceptable excipients, which can be any substance that does not itself induce the production of antibodies harmful to the patient receiving the composition, and which can be administered without undue toxicity. Depending on the formulation of the immunogenic compositions described below in more details, pharmaceutically acceptable excipients can include liquids such as water, saline, glycerol and ethanol. Auxiliary substances, such as wetting or emulsifying agents, pH buffering substances, and the like, can also be present in such formulations. A thorough discussion of suitable excipients is available in Gennaro (2000) Remington: The Science and Practice of Pharmacy. 20th edition, ISBN: 0683306472.

The immunogenic compositions are preferably sterile.

As mentioned above, the immunogenic composition against N. meningitidis serogroups A, C, W135, Y of this invention is formulated as a solid, for instance as a lyophilized (freeze-dried) product, and comprises one or more pharmaceutically acceptable excipients comprising one or more bulking agents, such as a sugar, said bulking agents being in a total amount ≤30 mg per unit dose of the composition of polysaccharide antigens in solid form.

The term “bulking agent” refers herein to an excipient compound or to mixtures of compounds that, when added to a solution intended for lyophilization, make up the bulk lyophilized product.

A pre-lyophilization solution of the polysaccharide antigens of this invention as the starting product for obtaining the solid, lyophilized composition above, comprises then one or more bulking agents, such as a sugar, in a total concentration of the bulking agent ≤6% by weight with respect to the total volume of the solution before lyophilization (indicated in the following also as pre-lyophilization solution or pre-lyophilization bulk formulation), referred to in the following as “w/v”. In an embodiment, the lyophilized composition comprises one or more bulking agents in a total concentration ≤5% w/v. The amount of bulking agent in the pre-lyophilization solution is maintained in the final solid formulation too.

Non-limiting examples of bulking agents according to this invention are sucrose, mannitol, trehalose, and mixtures thereof; preferably, the bulking agent is sucrose.

In an embodiment, the lyophilized composition of this invention comprises one single bulking agent, and preferably it is sucrose.

In one embodiment, the bulking agent is comprised in the solid composition of polysaccharide antigens conjugates in an amount ranging from about 10 mg to about 30 mg per unit dose of the composition of polysaccharide antigens conjugates in solid form. In an embodiment, the bulking agent is in an amount ranging from about 10 mg to about 25 mg. In other embodiments, the solid composition of polysaccharide antigens conjugates comprises an amount of the bulking agent from 12 mg to 30 mg, or from 12 mg to 25 mg, or from 12 mg to 20 mg, or from 12 mg to 15 mg, or from 12 mg to 13 mg. Preferably the amount of bulking agent in this composition is 12.5 mg per unit dose of the composition in solid form.

In one embodiment, the pre-lyophilization solution comprises the bulking agent at a concentration ranging from about 6% w/v to about 2% w/v, or from about 5% w/v to about 2% w/v, including a concentration of about 3% w/v, 4% w/v, 5% w/v with respect to the total volume of the pre-lyophilized solution. In other embodiments, the pre-lyophilization solution comprises the bulking agent at a concentration ranging from 6% w/v to 2.5% w/v, or from 5% w/v to 2.5% w/v, or from 4% w/V to 2.5% w/v, or from 3.5% w/v to 2.5% w/v. In an embodiment, the pre-lyophilization solution preferably comprises the bulking agent, preferably sucrose, at a concentration of about 3% w/v with respect to the total volume of the pre-lyophilization solution.

In a further preferred embodiment, the immunogenic composition against N. meningitidis serogroups A, C, W, Y in the pre-lyophilized liquid formulation of this invention comprises a buffering agent, for example a phosphate buffer at pH of about 7.2. This phosphate buffer may comprise potassium monobasic phosphate, to which potassium dibasic phosphate is added, e.g. K₂HPO₄.

A bulking agent and/or a buffering agent is preferably added to the composition and mixed to the antigens before lyophilization. In an embodiment, a phosphate buffer at a concentration of at least about 6 mM, at least 10 mM, or at least 40 mM, is added to the pre-lyophilization formulation.

In an embodiment, the immunogenic composition in the pre-lyophilized liquid formulation of this invention comprises the Men A, C, W135 and Y antigen conjugates, wherein the capsular saccharides and/or oligosaccharides thereof are conjugated to CRM₁₉₇ carrier, including recombinant CRM₁₉₇ carrier (rCRM₁₉₇), a tetanus toxoid carrier (TT), or a diphtheria toxoid carrier (DT). In an embodiment, the immunogenic composition in the pre-lyophilized liquid formulation of this invention comprises the Men A, C, W135 and Y antigen conjugates, wherein the capsular saccharides and/or oligosaccharides thereof are conjugated to CRM₁₉₇ carrier, including recombinant CRM₁₉₇ carrier (rCRM₁₉₇), or a tetanus toxoid carrier (TT). Preferably, the immunogenic composition in the pre-lyophilized liquid formulation of this invention comprises the Men A, C, W135 and Y antigen conjugates, wherein the capsular saccharides and/or oligosaccharides thereof are conjugated to CRM₁₉₇ carrier, including recombinant CRM₁₉₇ carrier (rCRM₁₉₇).

The immunogenic composition against N. meningitidis serogroup B used for the reconstitution of the Men ACW135Y immunogenic composition of this invention is formulated as a liquid “adsorbed” vaccine, with the protein antigens adsorbed onto an adsorbing agent, e.g. a compound containing aluminum, preferably selected from aluminum hydroxide, aluminum salts, and mixtures thereof; most preferably the absorbing agent is Alum (aluminum hydroxide). Non limiting examples of adsorbing agents containing aluminum are aluminum hydroxide (Alum), aluminum phosphate, potassium aluminum sulphate, oxyhydroxides and hydroxyphosphates (e.g. see chapters 8 & 9 of Vaccine Design . . . (1995) eds. Powell & Newman. ISBN: 030644867X. Plenum). The aluminum salts can take any suitable form (e.g. gel, crystalline, amorphous, etc.). In a most preferred embodiment, this Men B composition in liquid formulation is adjuvanted with Alum as adsorbing agent for the protein antigens. In an aspect, the absorbing agent, preferably Alum, is present at a concentration ranging from 2 to 5%, e.g. 3%, by weight with respect to the total volume of the present Men B antigens immunogenic composition in liquid form. Further concentrations of the absorbing agent for instance in the ranges from 2 to 4% or from 2.5 to 3.5% are intended to be part of this invention too.

The Men B liquid formulation may advantageously comprise one or more pharmaceutically acceptable tonicity modifying agents.

A “pharmaceutically acceptable tonicity modifying agent” is a compound that is physiologically tolerated and imparts a suitable tonicity to a formulation to prevent the net flow of water across cell membranes that are in contact with the formulation. In some embodiments, the tonicity modifying agent used for the composition is a salt (or mixtures of salts), preferably selected from sodium chloride, sugars, such as sucrose and sorbitol, and mixtures thereof. More preferably, the tonicity modifying agents in these compositions are mixtures of sodium chloride and sucrose; most preferably, they are aqueous solutions of sodium chloride and sucrose. Therefore, in one aspect of the present invention, sugars such as sucrose may be added to the present liquid formulation of Men B antigens as tonicity modifying agent, and they may also be added to the present pre-lyophilization solution of Men A, C, W135, Y antigens as bulking agents, as described above.

In a preferred embodiment, the liquid formulation of the immunogenic composition against N. meningitidis serogroup B used for preparing the reconstituted vaccine of this invention is an aqueous solution, having a concentration of sodium chloride up to about 3.8 mg/ml, preferably lower than about 3.0 mg/ml, more preferably of about 2.8 mg/ml. Values intermediate to the above said concentrations of sodium chloride are also intended to be part of this invention (e.g. 3.7, 3.6, 3.5, 3.4, 3.3, 3.2, 3.1, and 2.9 mg/ml), as well as values lower than 2.8 mg/ml. For example, the concentration of sodium chloride in the liquid formulation of the immunogenic composition against N. meningitidis serogroup B used for preparing the reconstituted vaccine of this invention may be between 0.1 mg/ml and 3.8 mg/ml, between 1 mg/ml and 3.8 mg/ml, between 1.5 mg/ml and 3.5 mg/ml, between 2 mg/ml and 3.5 mg/ml, between 2.5 mg/ml and 3.5 mg/ml, or between 2.5 mg/ml and 3.0 mg/ml.

The reconstituted vaccine is a liquid formulation, suitably an aqueous solution comprising sodium chloride. Preferably the reconstituted vaccine is a liquid formulation having a concentration of sodium chloride of less than 10 mg/ml, preferably 6.25 mg/ml.

In a further preferred embodiment, this immunogenic composition against N. meningitidis serogroup B comprises an aqueous solution having a concentration of sucrose in the range of about 2-3% by weight with respect to the total volume of the solution (referred to in the following as “w/v”), preferably of about 3% w/v. In further aspects of this invention, the concentration of sucrose in the immunogenic composition of Men B antigens may ranges from 2.0 mg/ml and 3.5 mg/ml, between 2.5 mg/ml and 3.5 mg/ml, or between 2.5 mg/ml and 3.0 mg/ml.

This immunogenic composition against N. meningitidis serogroup B can be moreover adjuvanted. Adjuvants which may be used in this composition include, but are not limited to insoluble metal salts, oil-in-water emulsions (e.g. MF59 or AS03, both containing squalene), saponins, non-toxic derivatives of LPS (such as monophosphoryl lipid A or 3-O-deacylated MPL), immunostimulatory oligonucleotides, detoxified bacterial ADP-ribosylating toxins, microparticles, liposomes, imidazoquinolines, or mixtures thereof. Other substances that act as immunostimulating agents are disclosed in chapter 7 of Vaccine Design . . . (1995) eds. Powell & Newman. ISBN: 030644867X. Plenum.

This immunogenic composition against N. meningitidis serogroup B in liquid formulation is preferably buffered e.g. at between pH 6 and pH 8, preferably around pH 6.5. An appropriate buffer may be selected from acetate, citrate, histidine, maleate, phosphate, succinate, tartrate and TRIS. Where a composition comprises an aluminum salt, it is preferred to use a histidine buffer [WO03/009869]. Histidine may be added to the composition in the form of the amino acid itself, preferably L-histidine, or in the form of a salt. The concentration of histidine in the composition may be typically of at least 1 μM up to 1M. In an embodiment, the concentration is at least 1 mM (e.g. at least 2 mM, 3 mM, 4 mM, 5 mM etc.) up to 250 mM (e.g. at most 200 mM, 150 mM, 100 mM, 50 mM, 40 mM, 30 mM, 20 mM, 10 mM, etc.). Preferably the concentration of histidine is between 2 mM and 20 mM (e.g. between 5 mM and 15 mM), most preferably it is about 10 mM.

In an embodiment, the reconstituted vaccine composition of the invention has a pharmaceutically acceptable osmolality to avoid cell distortion or lysis. A pharmaceutically acceptable osmolality will generally mean that solutions will have an osmolality, which is approximately isotonic or mildly hypertonic. In an embodiment, the reconstituted vaccine composition will have an osmolality in the range of 250 to 750 mOsm/kg, for example, the osmolality may be in the range of 250 to 550 mOsm/kg, such as in the range of 280 to 500 mOsm/kg. Suitably the liquid formulation of MenB antigens of the present invention will be slightly hypotonic, for example will have osmolality of about 210 mOsm/Kg, so that the reconstituted vaccine will reach the desired isotonicity or mild hypertonicity, as said above. Osmolality may be measured according to techniques known in the art, such as by means of a commercially available osmometer, for example the Advanced® Model 2020 available from Advanced Instruments Inc. (USA).

As shown in the experimental part below, the liquid formulation of MenB antigens and the solid formulation of the MenACW135Y antigens as described herein allow to obtain not only a safe, stable and easy to use vaccine product, but also to provide the vaccine in an effective formulation and for long-term storage. Characteristics of the present formulations include, but are not limited to, chemical stability of the immunogenic composition (e.g. proteolysis or fragmentation of proteins), physical/thermal stability of the immunogenic composition (e.g., aggregation, precipitation, adsorption), compatibility of the immunogenic composition with the container/closure system, interactions between immunogenic composition and inactive ingredients (e.g. buffers, salts, excipients, cryoprotectants), the manufacturing process, the dosage form (e.g., lyophilised, liquid), the environmental conditions encountered during shipping, storage and handling (e.g., temperature, humidity, shear forces), and the length of time between manufacture and usage.

Immunogenic compositions comprise an immunologically effective amount of the protein or conjugate of the invention, as well as any other components. By “immunologically effective amount”, it is meant that the administration of that amount to an individual, either as a single dose or as part of a series is effective for treatment or prevention. This amount varies depending on the health and physical condition of the individual to be treated, age, the degree of protection desired, the formulation of the vaccine and other relevant factors. It is expected that the amount will fall in a relatively broad range that can be determined through routine trials.

### Kit and Reconstituted Vaccine Composition

Subject of this invention is a kit comprising (i) a first container comprising an immunogenic composition against N. meningitidis serogroup B as described above, in liquid formulation; and (ii) a second container comprising an immunogenic composition against N. meningitidis serogroups A, C, W135, and Y as described above, in solid form. In the present kit, the first container and the second container can be containers separated from each other or they can form together a multi-container with separated parts for the liquid component and for the solid one.

In the present invention the liquid immunogenic composition in the first container is used to reconstitute the solid immunogenic composition in the second container, thus forming a vaccine composition comprising all antigens of both immunogenic compositions prior to administration. In an embodiment the reconstituted vaccine composition is a suspension.

In a preferred embodiment, the kit of this invention comprises (i) a pre-filled syringe as the first container and (ii) a vial as the second container.

In a particular embodiment, containers in the present kit are siliconized, to improve consistency of withdrawal of the vaccine upon reconstitution. Where an immunogenic composition of the invention is presented in a vial, this is preferably made of a glass or plastic material, more preferably this is made of siliconized glass or plastic material. The vial is preferably sterilized before the composition is added to it. The vial may include a single dose of vaccine, or it may include more than one dose (a ‘multidose’ vial) e.g. 10 doses. When using a multidose vial, each dose should be withdrawn with a sterile needle and syringe under strict aseptic conditions, taking care to avoid contaminating the vial contents. Preferred vials are made of colorless glass. A vial can have a rubber stopper adapted such that a pre-filled syringe, equipped with a needle, can be inserted into it, the contents of the syringe can be expelled into the vial (e.g. to reconstitute lyophilized material therein), and the contents of the vial can be withdrawn back into the syringe for administration to a patient.

In an embodiment, a same needle is used both for the reconstitution and for parenteral administration. In another embodiment, a needle already used for the reconstitution is removed prior to administration and replaced by another needle to be used for administration.

As showed by the results in the experimental part below, the liquid formulation of the Men B antigens composition guarantees that all relevant quality requirements of the final vaccine are met, in particular in terms of pH and osmolality of the vaccine composition, integrity and immunogenicity of each and all antigens. Moreover, following reconstitution of the solid formulation of MenACW135Y antigens, the present liquid formulation of MenB antigens has shown an optimal percentage of adsorption of all antigens onto the adsorbing agent.

### Administration

The present reconstituted vaccine composition will generally be administered directly to a patient by an appropriate route, for example by parenteral injection, e.g. intramuscularly. Intramuscular administration to the thigh or the upper arm is preferred. Injection may be via a needle (e.g. a hypodermic needle), but needle-free injection may alternatively be used.

The reconstituted vaccine composition of this invention may be administered to patients in unit doses, ranging between 0.1 and 1 ml, e.g. 0.5 ml. A typical intramuscular dose is of about 0.5 ml. Neisserial infections affect various areas of the body and so the compositions of the invention may be prepared in various forms. Compositions suitable for parenteral injection are most preferred.

The invention may be used to elicit systemic and/or mucosal immunity.

As used herein, a ‘dose’ of the composition is a volume of the composition suitable for administration to a subject as a single immunisation. Human vaccines are typically administered in a dosage volume of about 0.5 ml, although fractional doses may be administered (e.g., to children).

The composition may further be provided in a ‘multidose’ kit, i.e., a single container containing sufficient composition for multiple immunizations. Multidoses may include a preservative, or the multidose container may have an aseptic adaptor for removal of individual doses of the composition.

Administration can involve a single dose schedule or a multiple dose schedule. In this latter case, suitable intervals between priming doses can be routinely determined e.g. between 4-16 weeks, such as one month or two months.

The subject who is immunized is a human being, who may be any age e.g. 0-12 months old, 1-5 years old, 5-18 years old, 18-55 years old, or more than 55 years old. Preferably, the subject who is immunized is an adolescent (e.g. 12-18 years old) or an adult (18 years or older).

Optionally, the subject is an adolescent or adult who has been immunized against N. meningitidis in childhood (e.g. before 12 years of age), and who receives a booster dose of an immunogenic composition according to the invention.

### Use of the Immunogenic Composition and of the Reconstituted Vaccine of the Invention

The immunogenic composition and the reconstituted vaccine composition of this invention, as described above, are suitable for use in medicine, and in particular can be used to immunize a mammal against meningococcal A, B, C, W135 and/or Y infection or disease, such that recipients of the immunogenic composition mount an immune response, which provides protection against infection by, and/or against disease due to Neisseria meningitidis bacteria.

Therefore, the reconstituted vaccine according to the invention is used in prophylactic methods for immunizing subjects against infection and/or disease caused by Neisseria meningitidis. The immunogenic composition and the reconstituted vaccine may also be used in therapeutic methods (i.e. to treat Neisseria meningitidis infection).

The invention also provides a method for raising an immune response in vivo against Neisseria meningitidis infection in a mammal, comprising administering an immunogenic composition of the invention or the reconstituted vaccine to the mammal.

The immune response is preferably protective and preferably involves antibodies and/or cell-mediated immunity. Preferably, the immune response is a bactericidal antibody response. The method may raise a booster response. By raising an in vivo immune response, the mammal can be protected against Neisserial disease (in particular meningococcal infection).

The invention also provides a method for protecting a mammal against a Neisserial (e.g. meningococcal) infection, comprising administering to the mammal an immunogenic composition or the reconstituted vaccine of the invention.

The immunological composition of the invention is preferably formulated as vaccine products, which are suitable for therapeutic (i.e. to treat an infection) or prophylactic (i.e. to prevent an infection) use. Vaccines are typically prophylactic.

The mammal is preferably a human. The human may be an adult, an adolescent or a child (e.g. a toddler or infant). A vaccine intended for children may also be administered to adults e.g. to assess safety, dosage, immunogenicity, etc.

The uses and methods are particularly useful for preventing/treating diseases including, but not limited to, meningitis (particularly bacterial, such as meningococcal, meningitis) and bacteremia. For instance, they are suitable for active immunisation of individuals against invasive meningococcal disease caused by N. meningitidis, preferably caused by N. meningitidis serogroups A, B, C, W135, or Y.

Protection against N. meningitidis can be measured epidemiologically e.g. in a clinical trial, but it is convenient to use an indirect measure to confirm that an immunogenic composition elicits a serum bactericidal antibody (SBA) response in recipients. In the SBA assay, sera from recipients of the composition are incubated with target bacteria (in the present invention, N. meningitidis) in the presence of complement (preferably human complement, although baby rabbit complement is often used instead) and killing of the bacteria is assessed at various dilutions of the sera to determine SBA activity. Results observed in the SBA assay can be reinforced by carrying out a competitive SBA assay to provide further indirect evidence of the immunogenic activity of antigen(s) of interest. In the competitive SBA assay, sera from recipients of the immunogenic composition containing the antigen(s) are pre-incubated with said antigen(s), and subsequently incubated with target bacteria in the presence of human complement. Killing of the bacteria is then assessed, and they will be reduced or abolished if bactericidal antibodies in the recipients' sera bind to the antigens of interested during the pre-incubation phase and are therefore not available to bind to surface antigen on the bacteria.

It is not necessary that the composition should protect against each and every strain of N. meningitidis, or that each and every recipient of the composition must be protected. Such universal protection is not the normal standard in this field. Rather, protection is normally assessed against a panel of reference laboratory strains, often selected on a country-by-country basis, and perhaps varying with time and is measured across a population of recipients.

Preferred compositions of the invention can confer an antibody titer in a patient that is superior to the criterion for seroprotection for each antigenic component for an acceptable percentage of human subjects. Antigens with an associated antibody titer above which a host is considered to be seroconverted against the antigen are well known, and such titers are published by organizations, such as WHO. Preferably more than 80% of a statistically significant sample of subjects is seroconverted, more preferably more than 90%, still more preferably more than 93% and most preferably 96-100%.

Immunogenic compositions comprise an immunologically effective amount of immunogen, as well as any other of other specified components, as needed.

By ‘immunologically effective amount’, it is meant that the administration of that amount to an individual, either in a single dose or as part of a series, is effective for treatment or prevention.

The term “prevention” means that the progression of the disease is reduced and/or eliminated, or that the onset of the disease is eliminated. For example, the immune system of a subject may be primed (e.g. by vaccination) to trigger an immune response and repel infection such that the onset of the disease is eliminated.

A vaccinated subject may thus get infected but is better able to repel the infection than a control subject. This amount varies depending upon the health and physical condition of the individual to be treated, age, the taxonomic group of the individual to be treated (e.g. non-human primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

The composition may be administered in conjunction with other immunoregulatory agents.

### Amount of Antigens in the Compositions of the Invention

In terms of Men ACWY saccharides the present compositions may be formulated to give substantially a 1:1:1:1 ratio (measured as mass of saccharide), e.g., the mass of each serogroup's saccharide is within +10% of each other, though other ratios will not affect the compositions disclosed herein. As an alternative to a 1:1:1:1 ratio, a double dose of the serogroup A saccharide may be used (2:1:1:1).

In an embodiment, the present reconstituted vaccine comprises 5-15 μg/ml, e.g. 10 μg/ml, of each of MenC, MenW135, and MenY saccharide. In an embodiment, the present reconstituted vaccine comprises 10-30 μg/ml, e.g. 20 μg/ml, of the MenA saccharide.

As described above, the immunogenic composition in solid form of this invention preferably comprises the Men A, C, W135 and Y antigen in the form of conjugates, wherein the Men A, C, W135, and Y capsular saccharides and/or oligosaccharides thereof are conjugated to CRM₁₉₇ carrier, including recombinant CRM₁₉₇ carrier (rCRM₁₉₇). In an embodiment, a unit dose of 0.5 mL of the present reconstituted vaccine comprises 10 μg of the Men A saccharide conjugated to from 12.5 to 33.3 μg of CRM₁₉₇, 5 μg of the Men C saccharide conjugated to from 6.3 to 12.5 μg of CRM₁₉₇, 5 μg of Men W135 saccharide conjugated to from 3.3 to 10.0 μg of CRM₁₉₇, 5 μg of the Men Y saccharide conjugated to from 3.3 to 10.0 μg of CRM₁₉₇.

In an embodiment, the immunogenic composition in liquid formulation against N. meningitidis serogroup B infections comprises 40-60 μg/ml, e.g. 50 μg/ml, of the OMV antigen. In an embodiment, this immunogenic composition comprises 50-150 μg/ml, e.g. 100 μg/ml, of each protein antigen NHBA, fHbp, and NadA. In an embodiment, this immunogenic composition comprises 100-400 μg/ml, e.g. 100, 200, 300, or 400 μg/ml of the fusion fHbp polypeptide. In another embodiment, this immunogenic composition comprises 100-200 μg/ml of the fusion fHbp polypeptide, e.g. of the mutant v1.13 fHbp polypeptide.

In an embodiment, the present reconstituted vaccine comprises 40-60 μg/ml, e.g. 50 g/ml, of the OMV antigen. In an embodiment, the present reconstituted vaccine comprises 50-150 μg/ml, e.g. 100 μg/ml, of each protein antigen NHBA, fHbp, and NadA. In an embodiment, the present reconstituted vaccine comprises 100-400 μg/ml, e.g. 100, 200, 300, or 400 μg/ml of the fusion fHbp polypeptide. In another embodiment, this immunogenic composition comprises 100-200 μg/ml of the fusion fHbp polypeptide, e.g. of the mutant v1.13 fHbp polypeptide.

### Vaccine Efficacy

A reconstituted vaccine composition for use in the present invention preferably has a vaccine efficacy against each strain of N. meningitidis of at least 10% e.g. ≥20%, ≥30%, ≥40%, ≥50%, ≥60%, ≥70%, ≥80%, ≥85%, ≥90%, or more.

Vaccine efficacy is determined by the reduction in relative risk of developing meningococcal disease in subjects who receive a composition according to the invention compared to subjects who do not receive such a composition (e.g. are non-immunized or who receive a placebo or negative control). Thus, the incidence of meningococcal disease in a population which has been immunized according to the invention is compared to the incidence in a control population who has not been immunized according to the invention to give relative risk and vaccine efficacy is 100% minus this figure.

Vaccine efficacy is determined for a population rather than for an individual. Thus, it is a useful epidemiologic tool but does not predict individual protection. For instance, an individual subject might be exposed to a very large inoculum of the infecting agent or might have other risk factors which make them more subject to infection, but this does not negate the validity or utility of the efficacy measure. The size of a population which is immunized according to the invention, and for which vaccine efficacy is measured, is ideally at least 100 and may be higher e.g. at least 500 subjects. The size of the control group should also be at least 100 e.g. at least 500.

All references or patent applications cited within this specification are incorporated herein by reference.

### Aspects of the Invention are Summarized in the Following Numbered Paragraphs:

1. An immunogenic composition against N. meningitidis serogroups A, C, W135, and Y, comprising a composition of capsular polysaccharide antigens of the meningococcal serogroups A, C, W135, and Y conjugated to carrier protein(s) and one or more pharmaceutically acceptable excipients, wherein said composition is in a solid form and said excipients comprise one or more bulking agents in amount ≤30 mg per unit dose of the composition of polysaccharide antigens in solid form.
2. The immunogenic composition of paragraph 1, wherein said bulking agent is selected from the group consisting of sucrose, mannitol, trehalose, and mixtures thereof.
3. The immunogenic composition of paragraph 1 or 2, wherein said bulking agent is sucrose.
4. The immunogenic composition of any one of the preceding paragraphs, wherein the amount of said one or more bulking agents is in amount ≤25 mg per unit dose of the composition of polysaccharide antigens in solid form.
5. The immunogenic composition of any one of the preceding paragraphs, wherein the amount of said one or more bulking agents ranges between 10 mg and 30 mg, optionally between 10 mg and 25 mg, per unit dose of the composition of polysaccharide antigens in solid form.
6. The immunogenic composition of any one of the preceding paragraphs, wherein the amount of said one or more bulking agents is 12.5 mg per unit dose of the composition of polysaccharide antigens in solid form.
7. The immunogenic composition of any one of the preceding paragraphs, wherein said composition in a solid form is a lyophilized composition.
8. The immunogenic composition of any one of the preceding paragraphs, wherein said capsular polysaccharide antigens of the meningococcal serogroups A, C, W135, and Y are conjugated to CRM₁₉₇ as carrier protein.
9. A pre-lyophilization solution comprising a composition of capsular polysaccharide antigens of the meningococcal serogroups A, C, W135, and Y conjugated to carrier protein(s), one or more pharmaceutically acceptable excipients, and an aqueous solution of a buffering agent, wherein said excipients comprise one or more bulking agents in a concentration ≤6% w/v with respect to the total volume of the solution.
10. The pre-lyophilization solution of paragraph 9, wherein the concentration of said bulking agent is ≤5% w/v with respect to the total volume of the solution.
11. The pre-lyophilization solution of paragraph 9, wherein the concentration of said bulking agent ranges from 6% w/v to 2% w/v, optionally ranges from 5% w/v to 2% w/v, or is 3% w/v with respect to the total volume of the pre-lyophilized solution.
12. The pre-lyophilization solution of paragraphs 9 to 11, wherein said aqueous solution of a buffering agent is a phosphate buffer comprising potassium monobasic phosphate, to which potassium dibasic phosphate is added.
13. A reconstituted vaccine against N. meningitidis serogroups A, B, C, W135, and Y comprising the immunogenic composition against N. meningitidis serogroups A, C, W135, and Y in solid form as defined in paragraphs 1-8, reconstituted with a liquid formulation of an immunogenic composition against N. meningitidis serogroup B comprising one or more Men B antigens and an adsorbing agent for said antigens.
14. The reconstituted vaccine of paragraph 13, wherein said immunogenic composition against N. meningitidis serogroup B comprises a meningococcal NHBA antigen, a meningococcal NadA antigen, a meningococcal fHbp antigen, a meningococcal outer membrane vesicles (OMVs), and a fusion polypeptide comprising v1, v2 and v3 meningococcal fHbp polypeptides in the order v2-v3-v1 from N- to C-terminus.
15. The reconstituted vaccine of paragraph 14, wherein the v1 fHbp polypeptide is a mutant v1.13 fHbp polypeptide comprising an amino acid sequence having at least 80% sequence identity to SEQ ID NO: 16 wherein the amino acid sequence includes a substitution mutation at one or more of residues S216, E211 or E232 of SEQ ID NO: 16.
16. The reconstituted vaccine of any one of paragraphs 14 or 15, wherein the mutant v1.13 meningococcal fHbp polypeptide comprises an amino acid sequence that differs from SEQ ID NO: 16 by at least one or more of the substitutions S216R, E211A and E232A.
17. The reconstituted vaccine of paragraph 16, wherein the amino acid sequence comprises substitutions at multiple residues selected from the following:
   (i) E211A and S216R, and
   (ii) E211A and E232A.
18. The reconstituted vaccine of paragraph 14, wherein said meningococcal NHBA antigen and said meningococcal fHbp antigen are fusion proteins with meningococcal accessory proteins, e.g. a NHBA-GNA1030 fusion protein and a GNA2091-fHbp fusion protein.
19. The reconstituted vaccine of any one of paragraphs 13-18, wherein said liquid formulation is an aqueous solution comprising sodium chloride.
20. The reconstituted vaccine of paragraph 19, wherein said liquid formulation has a concentration of sodium chloride ≤3.8 mg/ml, preferably of 2.8 mg/ml.
21. The reconstituted vaccine of any one of paragraphs 13-20, wherein said adsorbent agent comprises a compound containing aluminium selected from aluminium hydroxide, aluminium salts and mixtures thereof; and preferably is Alum.
22. A kit comprising (i) a first container comprising the immunogenic composition against N. meningitidis serogroup B in a liquid form as defined in any one of paragraphs 13-21; and (ii) a second container comprising an immunogenic composition against N. meningitidis serogroups A, C, W135, and Y, in a solid form (e.g. freeze-dried) as defined in any one of paragraphs 1-8.
23. The kit of paragraph 22, wherein said first container is a prefilled syringe and said second container is a vial.
24. The kit of any one of paragraphs 22 or 23, wherein said containers or said multi-container are siliconized.
25. An immunogenic composition of any of paragraphs 1 to 8, or a reconstituted vaccine of any of paragraphs 11 to 19, for use as a vaccine.
26. An immunogenic composition of any of paragraphs 1 to 8, or a reconstituted vaccine of any of paragraphs 13 to 21, for use in a method in the prophylax is or treatment of infection and disease caused by N. meningitidis in a mammal, e.g. a human.
27. A method for preparing the reconstituted vaccine against N. meningitidis serogroups A, B, C, W135, and Y of any of paragraphs 13 to 21, comprising a step of reconstituting said immunogenic composition against N. meningitidis serogroups A, C, W135, and Y in solid form as defined in paragraphs 1-8, with a liquid formulation of an immunogenic composition against N. meningitidis serogroup B comprising one or more Men B antigens and an adsorbing agent for said Men B antigens, as defined in any one of paragraphs 14 to 21.
28. A method for the treatment or prevention of infection and disease caused by N. meningitidis in a subject in need thereof comprising administering to said subject a therapeutically effective amount of an immunogenic composition according to any of paragraphs 1 to 8, or of a reconstituted vaccine according to any of paragraphs 13 to 21.
29. A method of inducing an immune response to N. meningitidis in a subject, the method comprising administering a therapeutically or prophylactically effective amount of an immunogenic composition according to any of paragraphs 1 to 8 or of a reconstituted vaccine according to any of paragraphs 13 to 21.
30. Use of an immunogenic composition according to any of paragraphs 1 to 8 or of a reconstituted vaccine according to any of paragraphs 13 to 21 in the manufacture of a medicament for the treatment or prevention of an infection or disease caused by N. meningitidis.

It will be understood that the invention is described above by way of example only and modifications may be made whilst remaining within the scope and spirit of the invention.

### DESCRIPTION OF THE SEQUENCE LISTING

SEQ ID NO: 1 [fHbp protein from strain MC58]
CSSGGGGVAADIGAGLADALTAPLDHKDKGLQSLTLDQSVRKNEKLKLAAQGAEKTYGNGDSLNTGKLKNDKVS
RFDFIRQIEVDGQLITLESGEFQVYKQSHSALTAFQTEQIQDSEHSGKMVAKRQFRIGDIAGEHTSFDKLPEGGR
ATYRGTAFGSDDAGGKLTYTIDFAAKQGNGKIEHLKSPELNVDLAAADIKPDGKRHAVISGSVLYNQAEKGSYSL
GIFGGKAQEVAGSAEVKTVNGIRHIGLAAKQ
SEQ ID NO: 2 [fHbp protein from strain 961-5945]
CSSGGGGVAADIGAGLADALTAPLDHKDKSLQSLTLDQSVRKNEKLKLAAQGAEKTYGNGDSLNTGKLKNDKVS
RFDFIRQIEVDGQLITLESGEFQIYKQDHSAVVALQIEKINNPDKIDSLINQRSFLVSGLGGEHTAFNQLPDGKAE
YHGKAFSSDDAGGKLTYTIDFAAKQGHGKIEHLKTPEQNVELAAAELKADEKSHAVILGDTRYGSEEKGTYHLAL
FGDRAQEIAGSATVKIGEKVHEIGIAGKQ
SEQ ID NO: 3 [fHbp from strain M1239]
CSSGGGGSGGGGVAADIGTGLADALTAPLDHKDKGLKSLTLEDSIPQNGTLTLSAQGAEKTFKAGDKDNSLNTG
KLKNDKISRFDFVQKIEVDGQTITLASGEFQIYKQNHSAVVALQIEKINNPDKTDSLINQRSFLVSGLGGEHTAFN
QLPGGKAEYHGKAFSSDDPNGRLHYSIDFTKKQGYGRIEHLKTLEQNVELAAAELKADEKSHAVILGDTRYGSEE
KGTYHLALFGDRAQEIAGSATVKIGEKVHEIGIAGKQ
SEQ ID NO: 4 [NHBA wild-type from strain MC58]
MFKRSVIAMACIFALSACGGGGGGSPDVKSADTLSKPAAPVVSEKETEAKEDAPQAGSQGQGAPSAQGSQDMA
AVSEENTGNGGAVTADNPKNEDEVAQNDMPQNAAGTDSSTPNHTPDPNMLAGNMENQATDAGESSQPANQP
DMANAADGMQGDDPSAGGQNAGNTAAQGANQAGNNQAAGSSDPIPASNPAPANGGSNFGRVDLANGVLIDG
PSQNITLTHCKGDSCSGNNFLDEEVQLKSEFEKLSDADKISNYKKDGKNDKFVGLVADSVQMKGINQYIIFYKPK
PTSFARFRRSARSRRSLPAEMPLIPVNQADTLIVDGEAVSLTGHSGNIFAPEGNYRYLTYGAEKLPGGSYALRVQ
GEPAKGEMLAGAAVYNGEVLHFHTENGRPYPTRGRFAAKVDFGSKSVDGIIDSGDDLHMGTQKFKAAIDGNGF
KGTWTENGSGDVSGKFYGPAGEEVAGKYSYRPTDAEKGGFGVFAGKKEQD
SEQ ID NO: 5 [NadA from strain MC58]
MSMKHFPSKVLTTAILATFCSGALAATSDDDVKKAATVAIVAAYNNGQEINGFKAGETIYDIGEDGTITQKDATA
ADVEADDFKGLGLKKVVTNLTKTVNENKQNVDAKVKAAESEIEKLTTKLADTDAALADTDAALDETTNALNKLG
ENITTFAEETKTNIVKIDEKLEAVADTVDKHAEAFNDIADSLDETNTKADEAVKTANEAKQTAEETKQNVDAKVK
AAETAAGKAEAAAGTANTAADKAEAVAAKVTDIKADIATNKADIAKNSARIDSLDKNVANLRKETRQGLAEQAAL
SGLFQPYNVGRFNVTAAVGGYKSESAVAIGTGFRFTENFAAKAGVAVGTSSGSSAAYHVGVNYEW
SEQ ID NO: 6 [fusion protein of fHbp]
VAADIGAGLADALTAPLDHKDKGLQSLTLDQSVRKNEKLKLAAQGAEKTYGNGDSLNTGKLKNDKVSRFDFIRQ
IEVDGQLITLESGEFQVYKQSHSALTAFQTEQIQDSEHSGKMVAKRQFRIGDIAGEHTSFDKLPEGGRATYRGTA
FGSDDAGGKLTYTIDFAAKQGNGKIEHLKSPELNVDLAAADIKPDGKRHAVISGSVLYNQAEKGSYSLGIFGGKA
QEVAGSAEVKTVNGIRHIGLAAKQ
SEQ ID NO: 7 [GNA2091-fHbp fusion protein]
MVSAVIGSAAVGAKSAVDRRTTGAQTDDNVMALRIETTARSYLRQNNQTKGYTPQISVVGYDRHLLLLGQVATE
GEKQFVGQIARSEQAAEGVYNYITVASLPRTAGDIAGDTWNTSKVRATLLGISPATRARVKIVTYGNVTYVMGIL
TPEEQAQITQKVSTTVGVQKVITLYQNYVQRGSGGGGVAADIGAGLADALTAPLDHKDKGLQSLTLDQSVRKNE
KLKLAAQGAEKTYGNGDSLNTGKLKNDKVSRFDFIRQIEVDGQLITLESGEFQVYKQSHSALTAFQTEQIQDSEH
SGKMVAKRQFRIGDIAGEHTSFDKLPEGGRATYRGTAFGSDDAGGKLTYTIDFAAKQGNGKIEHLKSPELNVDL
AAADIKPDGKRHAVISGSVLYNQAEKGSYSLGIFGGKAQEVAGSAEVKTVNGIRHIGLAAKQ
SEQ ID NO: 8 [DG variant of NHBA]
SPDVKSADTLSKPAAPVVSEKETEAKEDAPQAGSQGQGAPSAQGGQDMAAVSEENTGNGGAAATDKPKNEDE
GAQNDMPQNAADTDSLTPNHTPASNMPAGNMENQAPDAGESEQPANQPDMANTADGMQGDDPSAGGENAG
NTAAQGTNQAENNQTAGSQNPASSTNPSATNSGGDFGRTNVGNSVVIDGPSQNITLTHCKGDSCSGNNFLDE
EVQLKSEFEKLSDADKISNYKKDGKNDGKNDKFVGLVADSVQMKGINQYIIFYKPKPTSFARFRRSARSRRSLPA
EMPLIPVNQADTLIVDGEAVSLTGHSGNIFAPEGNYRYLTYGAEKLPGGSYALRVQGEPSKGEMLAGTAVYNGEV
LHFHTENGRPSPSRGRFAAKVDFGSKSVDGIIDSGDGLHMGTQKFKAAIDGNGFKGTWTENGGGDVSGKFYGP
AGEEVAGKYSYRPTDAEKGGFGVFAGKKEQD
SEQ ID NO: 9 [NHBA-GNA1030 fusion]
MASPDVKSADTLSKPAAPVVSEKETEAKEDAPQAGSQGQGAPSAQGGQDMAAVSEENTGNGGAAATDKPKNE
DEGAQNDMPQNAADTDSLTPNHTPASNMPAGNMENQAPDAGESEQPANQPDMANTADGMQGDDPSAGGEN
AGNTAAQGTNQAENNQTAGSQNPASSTNPSATNSGGDFGRTNVGNSVVIDGPSQNITLTHCKGDSCSGNNFL
DEEVQLKSEFEKLSDADKISNYKKDGKNDGKNDKFVGLVADSVQMKGINQYIIFYKPKPTSFARFRRSARSRRSL
PAEMPLIPVNQADTLIVDGEAVSLTGHSGNIFAPEGNYRYLTYGAEKLPGGSYALRVQGEPSKGEMLAGTAVYNG
EVLHFHTENGRPSPSRGRFAAKVDFGSKSVDGIIDSGDGLHMGTQKFKAAIDGNGFKGTWTENGGGDVSGKFY
GPAGEEVAGKYSYRPTDAEKGGFGVFAGKKEQDGSGGGGATYKVDEYHANARFAIDHFNTSTNVGGFYGLTGS
VEFDQAKRDGKIDITIPVANLQSGSQHFTDHLKSADIFDAAQYPDIRFVSTKFNFNGKKLVSVDGNLTMHGKTAP
VKLKAEKFNCYQSPMAKTEVCGGDFSTTIDRTKWGVDYLVNVGMTKSVRIDIQIEAAKQ
SEQ ID NO: 10 [NadA fragment]
ATNDDDVKKAATVAIAAAYNNGQEINGFKAGETIYDIDEDGTITKKDATAADVEADDFKGLGLKKVVTNLTKTV
NENKQNVDAKVKAAESEIEKLTTKLADTDAALADTDAALDATTNALNKLGENITTFAEETKTNIVKIDEKLEAVAD
TVDKHAEAFNDIADSLDETNTKADEAVKTANEAKQTAEETKQNVDAKVKAAETAAGKAEAAAGTANTAADKAE
AVAAKVTDIKADIATNKDNIAKKANSADVYTREESDSKFVRIDGLNATTEKLDTRLASAEKSIADHDTRLNGLDK
TVSDLRKETRQGLAEQAALSGLFQPYNVG
SEQ ID NO: 11 [fHbp sequence]
CSSGSGSGGGGVAADIGTGLADALTAPLDHKDKGLKSLTLEDSISQNGTLTLSAQGAEKTFKVGDKDNSLNTGK
LKNDKISRFDFVQKIEVDGQTITLASGEFQIYKQDHSAVVALQIEKINNPDKIDSLINQRSFLVSGLGGEHTAFNQ
LPSGKAEYHGKAFSSDDAGGKLTYTIDFAAKQGHGKIEHLKTPEQNVELASAELKADEKSHAVILGDTRYGSEEK
GTYHLALFGDRAQEIAGSATVKIREKVHEIGIAGKQ
SEQ ID NO: 12 [fHbp sequence]
CSSGGGGSGGGGVTADIGTGLADALTAPLDHKDKGLKSLTLEDSISQNGTLTLSAQGAEKTYGNGDSLNTGKLK
NDKVSRFDFIRQIEVDGQLITLESGEFQVYKQSHSALTALQTEQEQDPEHSEKMVAKRRFRIGDIAGEHTSFDKL
PKDVMATYRGTAFGSDDAGGKLTYTIDFAAKQGHGKIEHLKSPELNVDLAVAYIKPDEKHHAVISGSVLYNQDEK
GSYSLGIFGEKAQEVAGSAEVETANGIHHIGLAAKQ
SEQ ID NO: 13 [GNA1030^{NL}]
ATYKVDEYHANARFAIDHFNTSTNVGGFYGLTGSVEFDQAKRDGKIDITIPIANLQSGSQHFTDHLKSADIFDAA
QYPDIRFVSTKFNFNGKKLVSVDGNLTMHGKTAPVKLKAEKFNCYQSPMEKTEVCGGDFSTTIDRTKWGMDYL
VNVGMTKSVRIDIQIEAAKQ
SEQ ID NO: 14 [GNA2091^{NL}]
VSAVIGSAAVGAKSAVDRRTTGAQTDDNVMALRIETTARSYLRQNNQTKGYTPQISVVGYNRHLLLLGQVATEG
EKQFVGQIARSEQAAEGVYNYITVASLPRTAGDIAGDTWNTSKVRATLLGISPATQARVKIVTYGNVTYVMGILT
PEEQAQITQKVSTTVGVQKVITLYQNYVQR
SEQ ID NO: 15 [v1.13 mature polypeptide from strain M982]
CSSGGGGVAADIGAGLADALTAPLDHKDKGLQSLTLDQSVRKNEKLKLAAQGAEKTYGNGDSLNTGKLKNDKVS
RFDFIRQIEVDGKLITLESGEFQVYKQSHSALTALQTEQVQDSEDSGKMVAKRQFRIGDIAGEHTSFDKLPKGGS
ATYRGTAFGSDDAGGKLTYTIDFAAKQGHGKIEHLKSPELNVELATAYIKPDEKRHAVISGSVLYNQDEKGSYSL
GIFGGQAQEVAGSAEVETANGIHHIGLAAKQ
SEQ ID NO: 16 [v1.13 ΔG]
VAADIGAGLADALTAPLDHKDKGLQSLTLDQSVRKNEKLKLAAQGAEKTYGNGDSLNTGKLKNDKVSRFDFIRQ
IEVDGKLITLESGEFQVYKQSHSALTALQTEQVQDSEDSGKMVAKRQFRIGDIAGEHTSFDKLPKGGSATYRGTA
FGSDDAGGKLTYTIDFAAKQGHGKIEHLKSPELNVELATAYIKPDEKRHAVISGSVLYNQDEKGSYSLGIFGGQA
QEVAGSAEVETANGIHHIGLAAKQ
SEQ ID NO: 17 [v1.13 ΔG (E211A/E232A)]
VAADIGAGLADALTAPLDHKDKGLQSLTLDQSVRKNEKLKLAAQGAEKTYGNGDSLNTGKLKNDKVSRFDFIRQ
IEVDGKLITLESGEFQVYKQSHSALTALQTEQVQDSEDSGKMVAKRQFRIGDIAGEHTSFDKLPKGGSATYRGTA
FGSDDAGGKLTYTIDFAAKQGHGKIEHLKSPELNVELATAYIKPDEKRHAVISGSVLYNQDAKGSYSLGIFGGQA
QEVAGSAAVETANGIHHIGLAAKQ
SEQ ID NO: 18 [v1.13 ΔG (E211A/S216R)]
VAADIGAGLADALTAPLDHKDKGLQSLTLDQSVRKNEKLKLAAQGAEKTYGNGDSLNTGKLKNDKVSRFDFIRQ
IEVDGKLITLESGEFQVYKQSHSALTALQTEQVQDSEDSGKMVAKRQFRIGDIAGEHTSFDKLPKGGSATYRGTA
FGSDDAGGKLTYTIDFAAKQGHGKIEHLKSPELNVELATAYIKPDEKRHAVISGSVLYNQDAKGSYRLGIFGGQA
QEVAGSAEVETANGIHHIGLAAKQ
SEQ ID NO: 19 [v1.15 mature polypeptide from strain NM452]
CSSGGGGSGGGGVAADIGAGLADALTAPLDHKDKGLKSLTLEDSISQNGTLTLSAQGAERTFKAGDKDNSLNTG
KLKNDKISRFDFIRQIEVDGQLITLESGEFQVYKQSHSALTALQTEQVQDSEHSGKMVAKRQFRIGDIVGEHTSF
GKLPKDVMATYRGTAFGSDDAGGKLTYTIDFAAKQGHGKIEHLKSPELNVDLAAADIKPDEKHHAVISGSVLYN
QAEKGSYSLGIFGGQAQEVAGSAEVETANGIRHIGLAAKQ
SEQ ID NO: 20 [v1.15 ΔG]
VAADIGAGLADALTAPLDHKDKGLKSLTLEDSISQNGTLTLSAQGAERTFKAGDKDNSLNTGKLKNDKISRFDFI
RQIEVDGQLITLESGEFQVYKQSHSALTALQTEQVQDSEHSGKMVAKRQFRIGDIVGEHTSFGKLPKDVMATYR
GTAFGSDDAGGKLTYTIDFAAKQGHGKIEHLKSPELNVDLAAADIKPDEKHHAVISGSVLYNQAEKGSYSLGIFG
GQAQEVAGSAEVETANGIRHIGLAAKQ
SEQ ID NO: 21 [v1.15 ΔG (S219R)]
VAADIGAGLADALTAPLDHKDKGLKSLTLEDSISQNGTLTLSAQGAERTFKAGDKDNSLNTGKLKNDKISRFDFI
RQIEVDGQLITLESGEFQVYKQSHSALTALQTEQVQDSEHSGKMVAKRQFRIGDIVGEHTSFGKLPKDVMATYR
GTAFGSDDAGGKLTYTIDFAAKQGHGKIEHLKSPELNVDLAAADIKPDEKHHAVISGSVLYNQAEKGSYRLGIFG
GQAQEVAGSAEVETANGIRHIGLAAKQ
SEQ ID NO: 22 [v1.15 ΔG (E214A/S219R)]
VAADIGAGLADALTAPLDHKDKGLKSLTLEDSISQNGTLTLSAQGAERTFKAGDKDNSLNTGKLKNDKISRFDFI
RQIEVDGQLITLESGEFQVYKQSHSALTALQTEQVQDSEHSGKMVAKRQFRIGDIVGEHTSFGKLPKDVMATYR
GTAFGSDDAGGKLTYTIDFAAKQGHGKIEHLKSPELNVDLAAADIKPDEKHHAVISGSVLYNQAAKGSYRLGIFG
GQAQEVAGSAEVETANGIRHIGLAAKQ
SEQ ID NO: 23 [v1.15 ΔG (E214A/E235A)]
VAADIGAGLADALTAPLDHKDKGLKSLTLEDSISQNGTLTLSAQGAERTFKAGDKDNSLNTGKLKNDKISRFDFI
RQIEVDGQLITLESGEFQVYKQSHSALTALQTEQVQDSEHSGKMVAKRQFRIGDIVGEHTSFGKLPKDVMATYR
GTAFGSDDAGGKLTYTIDFAAKQGHGKIEHLKSPELNVDLAAADIKPDEKHHAVISGSVLYNQAAKGSYSLGIFG
GQAQEVAGSAAVETANGIRHIGLAAKQ
SEQ ID NO: 24 [v2 wt from strain 2996]
MNRTAFCCLSLTAALILTACSSGGGGVAADIGAGLADALTAPLDHKDKSLQSLTLDQSVRKNEKLKLAAQGAEKT
YGNGDSLNTGKLKNDKVSRFDFIRQIEVDGQLITLESGEFQIYKQDHSAVVALQIEKINNPDKIDSLINQRSFLVS
GLGGEHTAFNQLPDGKAEYHGKAFSSDDAGGKLTYTIDFAAKQGHGKIEHLKTPEQNVELAAAELKADEKSHAV
ILGDTRYGSEEKGTYHLALFGDRAQEIAGSATVKIGEKVHEIGIAGKQ
SEQ ID NO: 25 [v2 mature polypeptide]
CSSGGGGVAADIGAGLADALTAPLDHKDKSLQSLTLDQSVRKNEKLKLAAQGAEKTYGNGDSLNTGKLKNDKVS
RFDFIRQIEVDGQLITLESGEFQIYKQDHSAVVALQIEKINNPDKIDSLINQRSFLVSGLGGEHTAFNQLPDGKAE
YHGKAFSSDDAGGKLTYTIDFAAKQGHGKIEHLKTPEQNVELAAAELKADEKSHAVILGDTRYGSEEKGTYHLAL
FGDRAQEIAGSATVKIGEKVHEIGIAGKQ
SEQ ID NO: 26 [v2 ΔG]
VAADIGAGLADALTAPLDHKDKSLQSLTLDQSVRKNEKLKLAAQGAEKTYGNGDSLNTGKLKNDKVSRFDFIRQI
EVDGQLITLESGEFQIYKQDHSAVVALQIEKINNPDKIDSLINQRSFLVSGLGGEHTAFNQLPDGKAEYHGKAFSS
DDAGGKLTYTIDFAAKQGHGKIEHLKTPEQNVELAAAELKADEKSHAVILGDTRYGSEEKGTYHLALFGDRAQEI
AGSATVKIGEKVHEIGIAGKQ
SEQ ID NO: 27 [v3 wt from strain M1239]
MNRTAFCCLSLTTALILTACSSGGGGSGGGGVAADIGTGLADALTAPLDHKDKGLKSLTLEDSIPQNGTLTLSAQ
GAEKTFKAGDKDNSLNTGKLKNDKISRFDFVQKIEVDGQTITLASGEFQIYKQNHSAVVALQIEKINNPDKTDSLI
NQRSFLVSGLGGEHTAFNQLPGGKAEYHGKAFSSDDPNGRLHYSIDFTKKQGYGRIEHLKTLEQNVELAAAELK
ADEKSHAVILGDTRYGSEEKGTYHLALFGDRAQEIAGSATVKIGEKVHEIGIAGKQ
SEQ ID NO: 28 [v3 mature]
CSSGGGGSGGGGVAADIGTGLADALTAPLDHKDKGLKSLTLEDSIPQNGTLTLSAQGAEKTFKAGDKDNSLNTG
KLKNDKISRFDFVQKIEVDGQTITLASGEFQIYKQNHSAVVALQIEKINNPDKTDSLINQRSFLVSGLGGEHTAFN
QLPGGKAEYHGKAFSSDDPNGRLHYSIDFTKKQGYGRIEHLKTLEQNVELAAAELKADEKSHAVILGDTRYGSEE
KGTYHLALFGDRAQEIAGSATVKIGEKVHEIGIAGKQ
SEQ ID NO: 29 [v3 ΔG]
VAADIGTGLADALTAPLDHKDKGLKSLTLEDSIPQNGTLTLSAQGAEKTFKAGDKDNSLNTGKLKNDKISRFDFV
QKIEVDGQTITLASGEFQIYKQNHSAVVALQIEKINNPDKTDSLINQRSFLVSGLGGEHTAFNQLPGGKAEYHGK
AFSSDDPNGRLHYSIDFTKKQGYGRIEHLKTLEQNVELAAAELKADEKSHAVILGDTRYGSEEKGTYHLALFGDR
AQEIAGSATVKIGEKVHEIGIAGKQ
SEQ ID NO: 30 [v2 ΔG S32V/L123R]
VAADIGAGLADALTAPLDHKDKSLQSLTLDQVVRKNEKLKLAAQGAEKTYGNGDSLNTGKLKNDKVSRFDFIRQ
IEVDGQLITLESGEFQIYKQDHSAVVALQIEKINNPDKIDSLINQRSFRVSGLGGEHTAFNQLPDGKAEYHGKAFS
SDDAGGKLTYTIDFAAKQGHGKIEHLKTPEQNVELAAAELKADEKSHAVILGDTRYGSEEKGTYHLALFGDRAQE
IAGSATVKIGEKVHEIGIAGKQ
SEQ ID NO: 31 [v3 ΔG S32V/L126R]
VAADIGTGLADALTAPLDHKDKGLKSLTLEDVIPQNGTLTLSAQGAEKTFKAGDKDNSLNTGKLKNDKISRFDFV
QKIEVDGQTITLASGEFQIYKQNHSAVVALQIEKINNPDKTDSLINQRSFRVSGLGGEHTAFNQLPGGKAEYHGK
AFSSDDPNGRLHYSIDFTKKQGYGRIEHLKTLEQNVELAAAELKADEKSHAVILGDTRYGSEEKGTYHLALFGDR
AQEIAGSATVKIGEKVHEIGIAGKQ
SEQ ID NO: 32 [(23S_1.13_E211A/E232A)]
VAADIGAGLADALTAPLDHKDKSLQSLTLDQVVRKNEKLKLAAQGAEKTYGNGDSLNTGKLKNDKVSRFDFIRQ
IEVDGQLITLESGEFQIYKQDHSAVVALQIEKINNPDKIDSLINQRSFRVSGLGGEHTAFNQLPDGKAEYHGKAFS
SDDAGGKLTYTIDFAAKQGHGKIEHLKTPEQNVELAAAELKADEKSHAVILGDTRYGSEEKGTYHLALFGDRAQE
IAGSATVKIGEKVHEIGIAGKQGSGGGGVAADIGTGLADALTAPLDHKDKGLKSLTLEDVIPQNGTLTLSAQGA
EKTFKAGDKDNSLNTGKLKNDKISRFDFVQKIEVDGQTITLASGEFQIYKQNHSAVVALQIEKINNPDKTDSLIN
QRSFRVSGLGGEHTAFNQLPGGKAEYHGKAFSSDDPNGRLHYSIDFTKKQGYGRIEHLKTLEQNVELAAAELKA
DEKSHAVILGDTRYGSEEKGTYHLALFGDRAQEIAGSATVKIGEKVHEIGIAGKQGSGGGGVAADIGAGLADAL
TAPLDHKDKGLQSLTLDQSVRKNEKLKLAAQGAEKTYGNGDSLNTGKLKNDKVSRFDFIRQIEVDGKLITLESGE
FQVYKQSHSALTALQTEQVQDSEDSGKMVAKRQFRIGDIAGEHTSFDKLPKGGSATYRGTAFGSDDAGGKLTY
TIDFAAKQGHGKIEHLKSPELNVELATAYIKPDEKRHAVISGSVLYNQDAKGSYSLGIFGGQAQEVAGSAAVETA
NGIHHIGLAAKQ
SEQ ID NO: 33 [23S_1.13_E211A/S216R]
VAADIGAGLADALTAPLDHKDKSLQSLTLDQVVRKNEKLKLAAQGAEKTYGNGDSLNTGKLKNDKVSRFDFIRQ
IEVDGQLITLESGEFQIYKQDHSAVVALQIEKINNPDKIDSLINQRSFRVSGLGGEHTAFNQLPDGKAEYHGKAFS
SDDAGGKLTYTIDFAAKQGHGKIEHLKTPEQNVELAAAELKADEKSHAVILGDTRYGSEEKGTYHLALFGDRAQE
IAGSATVKIGEKVHEIGIAGKQGSGGGGVAADIGTGLADALTAPLDHKDKGLKSLTLEDVIPQNGTLTLSAQGA
EKTFKAGDKDNSLNTGKLKNDKISRFDFVQKIEVDGQTITLASGEFQIYKQNHSAVVALQIEKINNPDKTDSLIN
QRSFRVSGLGGEHTAFNQLPGGKAEYHGKAFSSDDPNGRLHYSIDFTKKQGYGRIEHLKTLEQNVELAAAELKA
DEKSHAVILGDTRYGSEEKGTYHLALFGDRAQEIAGSATVKIGEKVHEIGIAGKQGSGGGGVAADIGAGLADAL
TAPLDHKDKGLQSLTLDQSVRKNEKLKLAAQGAEKTYGNGDSLNTGKLKNDKVSRFDFIRQIEVDGKLITLESGE
FQVYKQSHSALTALQTEQVQDSEDSGKMVAKRQFRIGDIAGEHTSFDKLPKGGSATYRGTAFGSDDAGGKLTY
TIDFAAKQGHGKIEHLKSPELNVELATAYIKPDEKRHAVISGSVLYNQDAKGSYRLGIFGGQAQEVAGSAEVETA
NGIHHIGLAAKQ
SEQ ID NO: 34 [23S_1.15_S219R]
VAADIGAGLADALTAPLDHKDKSLQSLTLDQVVRKNEKLKLAAQGAEKTYGNGDSLNTGKLKNDKVSRFDFIRQ
IEVDGQLITLESGEFQIYKQDHSAVVALQIEKINNPDKIDSLINQRSFRVSGLGGEHTAFNQLPDGKAEYHGKAFS
SDDAGGKLTYTIDFAAKQGHGKIEHLKTPEQNVELAAAELKADEKSHAVILGDTRYGSEEKGTYHLALFGDRAQE
IAGSATVKIGEKVHEIGIAGKQGSGGGGVAADIGTGLADALTAPLDHKDKGLKSLTLEDVIPQNGTLTLSAQGA
EKTFKAGDKDNSLNTGKLKNDKISRFDFVQKIEVDGQTITLASGEFQIYKQNHSAVVALQIEKINNPDKTDSLIN
QRSFRVSGLGGEHTAFNQLPGGKAEYHGKAFSSDDPNGRLHYSIDFTKKQGYGRIEHLKTLEQNVELAAAELKA
DEKSHAVILGDTRYGSEEKGTYHLALFGDRAQEIAGSATVKIGEKVHEIGIAGKQGSGGGGVAADIGAGLADAL
TAPLDHKDKGLKSLTLEDSISQNGTLTLSAQGAERTFKAGDKDNSLNTGKLKNDKISRFDFIRQIEVDGQLITLES
GEFQVYKQSHSALTALQTEQVQDSEHSGKMVAKRQFRIGDIVGEHTSFGKLPKDVMATYRGTAFGSDDAGGKL
TYTIDFAAKQGHGKIEHLKSPELNVDLAAADIKPDEKHHAVISGSVLYNQAEKGSYRLGIFGGQAQEVAGSAEVE
TANGIRHIGLAAKQ
SEQ ID NO: 35 [23S_1.15_E214A/S219R]
VAADIGAGLADALTAPLDHKDKSLQSLTLDQVVRKNEKLKLAAQGAEKTYGNGDSLNTGKLKNDKVSRFDFIRQ
IEVDGQLITLESGEFQIYKQDHSAVVALQIEKINNPDKIDSLINQRSFRVSGLGGEHTAFNQLPDGKAEYHGKAFS
SDDAGGKLTYTIDFAAKQGHGKIEHLKTPEQNVELAAAELKADEKSHAVILGDTRYGSEEKGTYHLALFGDRAQE
IAGSATVKIGEKVHEIGIAGKQGSGGGGVAADIGTGLADALTAPLDHKDKGLKSLTLEDVIPQNGTLTLSAQGA
EKTFKAGDKDNSLNTGKLKNDKISRFDFVQKIEVDGQTITLASGEFQIYKQNHSAVVALQIEKINNPDKTDSLIN
QRSFRVSGLGGEHTAFNQLPGGKAEYHGKAFSSDDPNGRLHYSIDFTKKQGYGRIEHLKTLEQNVELAAAELKA
DEKSHAVILGDTRYGSEEKGTYHLALFGDRAQEIAGSATVKIGEKVHEIGIAGKQGSGGGGVAADIGAGLADAL
TAPLDHKDKGLKSLTLEDSISQNGTLTLSAQGAERTFKAGDKDNSLNTGKLKNDKISRFDFIRQIEVDGQLITLES
GEFQVYKQSHSALTALQTEQVQDSEHSGKMVAKRQFRIGDIVGEHTSFGKLPKDVMATYRGTAFGSDDAGGKL
TYTIDFAAKQGHGKIEHLKSPELNVDLAAADIKPDEKHHAVISGSVLYNQAAKGSYRLGIFGGQAQEVAGSAEVE
TANGIRHIGLAAKQ
SEQ ID NO: 36 [23S_1.15_E214A/E235A]
VAADIGAGLADALTAPLDHKDKSLQSLTLDQVVRKNEKLKLAAQGAEKTYGNGDSLNTGKLKNDKVSRFDFIRQ
IEVDGQLITLESGEFQIYKQDHSAVVALQIEKINNPDKIDSLINQRSFRVSGLGGEHTAFNQLPDGKAEYHGKAFS
SDDAGGKLTYTIDFAAKQGHGKIEHLKTPEQNVELAAAELKADEKSHAVILGDTRYGSEEKGTYHLALFGDRAQE
IAGSATVKIGEKVHEIGIAGKQGSGGGGVAADIGTGLADALTAPLDHKDKGLKSLTLEDVIPQNGTLTLSAQGA
EKTFKAGDKDNSLNTGKLKNDKISRFDFVQKIEVDGQTITLASGEFQIYKQNHSAVVALQIEKINNPDKTDSLIN
QRSFRVSGLGGEHTAFNQLPGGKAEYHGKAFSSDDPNGRLHYSIDFTKKQGYGRIEHLKTLEQNVELAAAELKA
DEKSHAVILGDTRYGSEEKGTYHLALFGDRAQEIAGSATVKIGEKVHEIGIAGKQGSGGGGVAADIGAGLADAL
TAPLDHKDKGLKSLTLEDSISQNGTLTLSAQGAERTFKAGDKDNSLNTGKLKNDKISRFDFIRQIEVDGQLITLES
GEFQVYKQSHSALTALQTEQVQDSEHSGKMVAKRQFRIGDIVGEHTSFGKLPKDVMATYRGTAFGSDDAGGKL
TYTIDFAAKQGHGKIEHLKSPELNVDLAAADIKPDEKHHAVISGSVLYNQAAKGSYSLGIFGGQAQEVAGSAAVE
TANGIRHIGLAAKQ
SEQ ID NO: 37 [v1.1 ΔG + His tag]
VAADIGAGLADALTAPLDHKDKGLQSLTLDQSVRKNEKLKLAAQGAEKTYGNGDSLNTGKLKNDKVSRFDFIRQ
IEVDGQLITLESGEFQVYKQSHSALTAFQTEQIQDSEHSGKMVAKRQFRIGDIAGEHTSFDKLPEGGRATYRGTA
FGSDDAGGKLTYTIDFAAKQGNGKIEHLKSPELGLAAKQLNVDLAAADIKPDGKRHAVISGSVLYNQAEKGSYSL
GIFGGKAQEVAGSAEVKTVNGIRHLEHHHHHH
SEQ ID NO: 38 [v1.13 ΔG + His tag]
VAADIGAGLADALTAPLDHKDKGLQSLTLDQSVRKNEKLKLAAQGAEKTYGNGDSLNTGKLKNDKVSRFDFIRQ
IEVDGKLITLESGEFQVYKQSHSALTALQTEQVQDSEDSGKMVAKRQFRIGDIAGEHTSFDKLPKGGSATYRGTA
FGSDDAGGKLTYTIDFAAKQGHGKIEHLKSPELNVELATAYIKPDEKRHAVISGSVLYNQDEKGSYSLGIFGGQA
QEVAGSAEVETANGIHHIGLAAKQLEHHHHHH
SEQ ID NO: 39 [v1.13 ΔG (E211A)]
VAADIGAGLADALTAPLDHKDKGLQSLTLDQSVRKNEKLKLAAQGAEKTYGNGDSLNTGKLKNDKVSRFDFIRQ
IEVDGKLITLESGEFQVYKQSHSALTALQTEQVQDSEDSGKMVAKRQFRIGDIAGEHTSFDKLPKGGSATYRGTA
FGSDDAGGKLTYTIDFAAKQGHGKIEHLKSPELNVELATAYIKPDEKRHAVISGSVLYNQDAKGSYSLGIFGGQA
QEVAGSAEVETANGIHHIGLAAKQLEHHHHHH
SEQ ID NO: 40 [v1.13 ΔG (S216R)]
VAADIGAGLADALTAPLDHKDKGLQSLTLDQSVRKNEKLKLAAQGAEKTYGNGDSLNTGKLKNDKVSRFDFIRQ
IEVDGKLITLESGEFQVYKQSHSALTALQTEQVQDSEDSGKMVAKRQFRIGDIAGEHTSFDKLPKGGSATYRGTA
FGSDDAGGKLTYTIDFAAKQGHGKIEHLKSPELNVELATAYIKPDEKRHAVISGSVLYNQDEKGSYRLGIFGGQA
QEVAGSAEVETANGIHHIGLAAKQLEHHHHHH
SEQ ID NO: 41 [v1.15 ΔG + His tag]
VAADIGAGLADALTAPLDHKDKGLKSLTLEDSISQNGTLTLSAQGAERTFKAGDKDNSLNTGKLKNDKISRFDFI
RQIEVDGQLITLESGEFQVYKQSHSALTALQTEQVQDSEHSGKMVAKRQFRIGDIVGEHTSFGKLPKDVMATYR
GTAFGSDDAGGKLTYTIDFAAKQGHGKIEHLKSPELNVDLAAADIKPDEKHHAVISGSVLYNQAEKGSYSLGIFG
GQAQEVAGSAEVETANGIRHIGLAAKQLEHHHHHH
SEQ ID NO: 42 [v1.15 ΔG (E214A) + His tag]
VAADIGAGLADALTAPLDHKDKGLKSLTLEDSISQNGTLTLSAQGAERTFKAGDKDNSLNTGKLKNDKISRFDFI
RQIEVDGQLITLESGEFQVYKQSHSALTALQTEQVQDSEHSGKMVAKRQFRIGDIVGEHTSFGKLPKDVMATYR
GTAFGSDDAGGKLTYTIDFAAKQGHGKIEHLKSPELNVDLAAADIKPDEKHHAVISGSVLYNQAAKGSYSLGIFG
GQAQEVAGSAEVETANGIRHIGLAAKQLEHHHHHH
SEQ ID NO: 43 [fHbp 231 wt fusion polypeptide]
VAADIGAGLADALTAPLDHKDKSLQSLTLDQSVRKNEKLKLAAQGAEKTYGNGDSLNTGKLKNDKVSFDFIRQIE
VDGQLITLESGEFQIYKQDHSAVVALQIEKINNPDKIDSLINQRSFLVSGLGGEHTAFNQLPDGKAEYHGKAFSS
DDAGGKLTYTIDFAAKQGHGKIEHLKTPEQNVELAAAELKADEKSHAVILGDTRYGSEEKGTYHLALFGDRAQEI
AGSATVKIGEKVHEIGIAGKQGSGGGGVAADIGTGLADALTAPLDHKDKGLKSLTLEDSIPQNGTLTLSAQGAEK
TFKAGDKDNSLNTGKLKNDKISRFDFVQKIEVDGQTITLASGEFQIYKQNHSAVVALQIEKINNPDKTDSLINQR
SFLVSGLGGEHTAFNQLPGGKAEYHGKAFSSDDPNGRLHYSIDFTKKQGYGRIEHLKTLEQNVELAAAELKADEK
SHAVILGDTRYGSEEKGTYHLALFGDRAQEIAGSATVKIGEKVHEIGIAGKQGSGGGGVAADIGAGLADALTAPL
DHKDKGLQSLTLDQSVRKNEKLKLAAQGAEKTYGNGSLNTGKLKNDKVSRFDFIRQIEVDGQLITLESGEFQVYK
QSHSALTAFQTEQIQDSEHSGKMVAKRQFRIGDIAGEHTSFDKLPEGGRATYRGTAFGSDDAGGKLTYTIDFAA
KQGNGKIEHLKSPELNVDLAAAIKPDGKRHAVISGSVLYNQAEKGSYSLGIFGKAQEVAGSAEVKTVNGIRHIGLA
AKQ
SEQ ID NO: 44 [fHbp 231S fusion polypeptide]
VAADIGAGLADALTAPLDHKDKSLQSLTLDQVVRKNEKLKLAAQGAEKTYGNGDSLNTGKLKNDKVSFDFIRQIE
VDGQLITLESGEFQIYKQDHSAVVALQIEKINNPDKIDSLINQRSFRVSGLGGEHTAFNQLPDGKAEYHGKAFSS
DDAGGKLTYTIDFAAKQGHGKIEHLKTPEQNVELAAAELKADEKSHAVILGDTRYGSEEKGTYHLALFGDRAQEI
AGSATVKIGEKVHEIGIAGKQGSGGGGVAADIGTGLADALTAPLDHKDKGLKSLTLEDVIPQNGTLTLSAQGAEK
TFKAGDKDNSLNTGKLKNDKISRFDFVQKIEVDGQTITLASGEFQIYKQNHSAVVALQIEKINNPDKTDSLINQR
SFRVSGLGGEHTAFNQLPGGKAEYHGKAFSSDDPNGRLHYSIDFTKKQGYGRIEHLKTLEQNVELAAAELKADE
KSHAVILGDTRYGSEEKGTYHLALFGDRAQEIAGSATVKIGEKVHEIGIAGKQGSGGGGVAADIGAGLADALTAP
LDHKDKGLQSLTLDQSVSKNEKLKLAAQGAEKTYGNGSLNTGKLKNDKVSRFDFIRQIEVDGQLITLESGEFQVY
KQSHSALTAFQTEQIQDSEHSGKMVAKRQFRIGDIAGEHTSFDKLPEGGRATYRGTAFGSDDAGGKLTYTIDFA
AKQGNGKIEHLKSPELNVDLAAAIKPDGKRHAVISGSVLYNQAEKGSYSLGIFGKAQEVAGSAEVKTVNGIRHIGL
AAKQ
SEQ ID NO: 45 [v1.13 full-length wt sequence]
MNRTAFCCFSLTAALILTACSSGGGGVAADIGAGLADALTAPLDHKDKGLQSLTLDQSVRKNEKLKLAAQGAEKT
YGNGDSLNTGKLKNDKVSRFDFIRQIEVDGKLITLESGEFQVYKQSHSALTALQTEQVQDSEDSGKMVAKRQFR
IGDIAGEHTSFDKLPKGGSATYRGTAFGSDDAGGKLTYTIDFAAKQGHGKIEHLKSPELNVELATAYIKPDEKRH
AVISGSVLYNQDEKGSYSLGIFGGQAQEVAGSAEVETANGIHHIGLAAKQ
SEQ ID NO: 46 [v1.15 full-length wt sequence]
MNRTTFCCLSLTAALILTACSSGGGGSGGGGVAADIGAGLADALTAPLDHKDKGLKSLTLEDSISQNGTLTLSAQ
GAERTFKAGDKDNSLNTGKLKNDKISRFDFIRQIEVDGQLITLESGEFQVYKQSHSALTALQTEQVQDSEHSGK
MVAKRQFRIGDIVGEHTSFGKLPKDVMATYRGTAFGSDDAGGKLTYTIDFAAKQGHGKIEHLKSPELNVDLAAA
DIKPDEKHHAVISGSVLYNQAEKGSYSLGIFGGQAQEVAGSAEVETANGIRHIGLAAKQ
SEQ ID NO: 47 [mature fHbp v1.1]
CSSGGGGVAADIGAGLADALTAPLDHKDKGLQSLTLDQSVRKNEKLKLAAQGAEKTYGNGDSLNTGKLKNDKVS
RFDFIQIEVDGQLITLESGEFQVYKQSHSALTAFQTEQIQDSEHSGKMVAKRQFRIGDIAGEHTSFDKLPEGGRA
TYRGTAFGSDDAGGKLTYTIDFAAKQGNGKIEHLKSPELNVDLAAADIKPDGKRHAVISGSVLYNQAEKGSYSLG
IFGGKAQEVAGSAEVKTVNGIRHIGLAAKQ
SEQ ID NO: 48 [optional N-terminal amino acid sequence]
MGPDSDRLQQRR
SEQ ID NO: 49 [SEQ ID NO: 48 + SEQ ID NO: 33; 23S_1.13_E211A/S216R with additional
N-terminal amino acid sequence]
MGPDSDRLQQRRVAADIGAGLADALTAPLDHKDKSLQSLTLDQVVRKNEKLKLAAQGAEKTYGNGDSLNTGKL
KNDKVSRFDFIRQIEVDGQLITLESGEFQIYKQDHSAVVALQIEKINNPDKIDSLINQRSFRVSGLGGEHTAFNQL
PDGKAEYHGKAFSSDDAGGKLTYTIDFAAKQGHGKIEHLKTPEQNVELAAAELKADEKSHAVILGDTRYGSEEKG
TYHLALFGDRAQEIAGSATVKIGEKVHEIGIAGKQGSGGGGVAADIGTGLADALTAPLDHKDKGLKSLTLEDVIP
QNGTLTLSAQGAEKTFKAGDKDNSLNTGKLKNDKISRFDFVQKIEVDGQTITLASGEFQIYKQNHSAVVALQIEK
INNPDKTDSLINQRSFRVSGLGGEHTAFNQLPGGKAEYHGKAFSSDDPNGRLHYSIDFTKKQGYGRIEHLKTLE
QNVELAAAELKADEKSHAVILGDTRYGSEEKGTYHLALFGDRAQEIAGSATVKIGEKVHEIGIAGKQGSGGGGV
AADIGAGLADALTAPLDHKDKGLQSLTLDQSVRKNEKLKLAAQGAEKTYGNGDSLNTGKLKNDKVSRFDFIRQIE
VDGKLITLESGEFQVYKQSHSALTALQTEQVQDSEDSGKMVAKRQFRIGDIAGEHTSFDKLPKGGSATYRGTAF
GSDDAGGKLTYTIDFAAKQGHGKIEHLKSPELNVELATAYIKPDEKRHAVISGSVLYNQDAKGSYRLGIFGGQAQ
EVAGSAEVETANGIHHIGLAAKQ
SEQ ID NO: 50 [linker]
GGSG
SEQ ID NO: 51 [linker]
GGSGG
SEQ ID NO: 52 [linker]
GSGSG
SEQ ID NO: 53 [linker]
GSGGG
SEQ ID NO: 54 [linker]
GGGSG
SEQ ID NO: 55 [linker]
GSSSG
SEQ ID NO: 56 [linker]
GSGGGG

### MODES FOR CARRYING OUT THE INVENTION

The invention will now be further defined by reference to the following non-limiting examples.

### EXAMPLES

### Example 1: Lyophilization of MenACW135Y Antigens Composition

Lyophilization allows obtaining the title vaccine components in a stable form over long-term storage, suitable for reconstitution with an aqueous solution containing other vaccine's components prior to administration.

For the lyophilization process of MenACW135Y antigens composition, addition of the bulking agent excipient was tested to evaluate the potential impact of excipient addition on the lyophilization process and on the composition itself. As shown below, a surprising result was obtained by adding an amount of bulking agent, in particular sucrose, to the pre-lyophilization bulk formulation that was much reduced with respect to inventors' experience with similar vaccines. This reduced amount of bulking agent below 6% w/v in the pre-lyo bulk solution, together with an appropriate phosphate buffering agent, as detailed below, provided good results in terms of antigen aggregation and of residual moisture % (RM %) in the lyophilized product, verified under accelerated stability conditions.

To this end, three freeze-dried products have been prepared differing from one another only for the sucrose concentration in the pre-lyo bulk formulation of MenACW135Y antigens, namely a “Formulation 0” with sucrose at 2% w/v, a “Formulation 1” with sucrose at 3% w/v and a “Formulation 2” with sucrose at 5% w/v. A 10 mM phosphate buffer concentration was applied to the tested pre-lyo bulk formulations, whose composition was as summarized below in Table 1:

TABLE 1
Composition of MenACW135Y antigens pre-lyo bulk formulations

| | Concentration in pre-lyo bulk formulations | | |
|---|---|---|---|
| Component | Formulation 0 | Formulation 1 | Formulation 2 |
| MenA-CRM₁₉₇ (μg/ml) | 24 | 24 | 24 |
| MenC-CRM₁₉₇ (μg/ml) | 12 | 12 | 12 |
| MenW135-CRM₁₉₇ (μg/ml) | 12 | 12 | 12 |
| MenY-CRM₁₉₇ (μg/ml) | 12 | 12 | 12 |
| Sucrose (% w/v) | 2 | 3 | 5 |
| K₂HPO₄ (mM) | 10 | 10 | 10 |

The pH of the three formulations was adjusted to a final pH value of 7.2 by adding KOH into the buffers.

The batches were freeze-dried within the same lyophilization cycle, stored at 2-8° C. and then incubated at 37° C. with 75% of residual humidity (RH) and at 25° C. with 60% RH approximately 2 weeks after the production day.

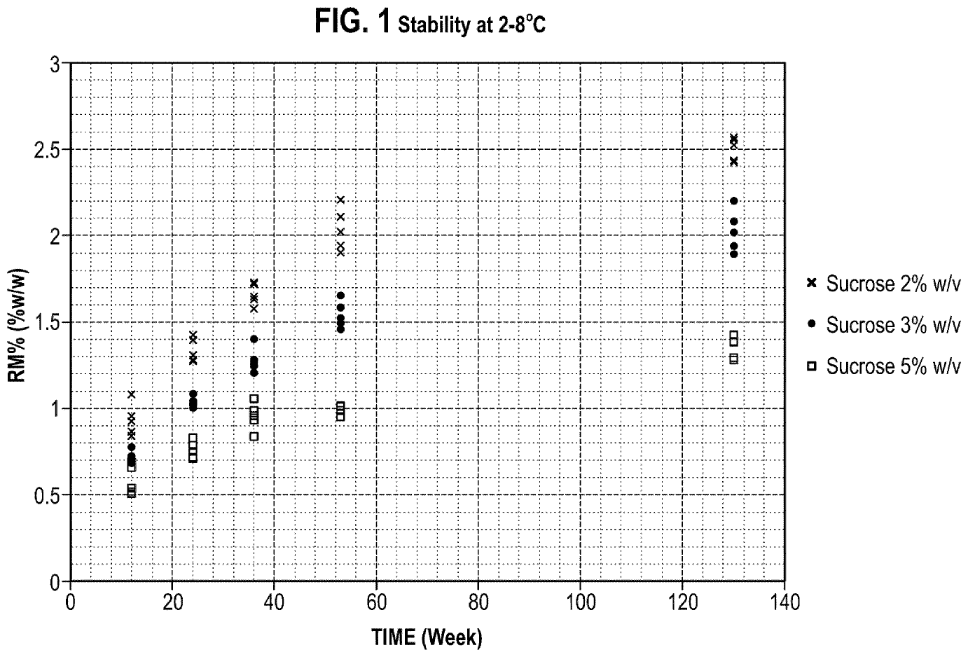

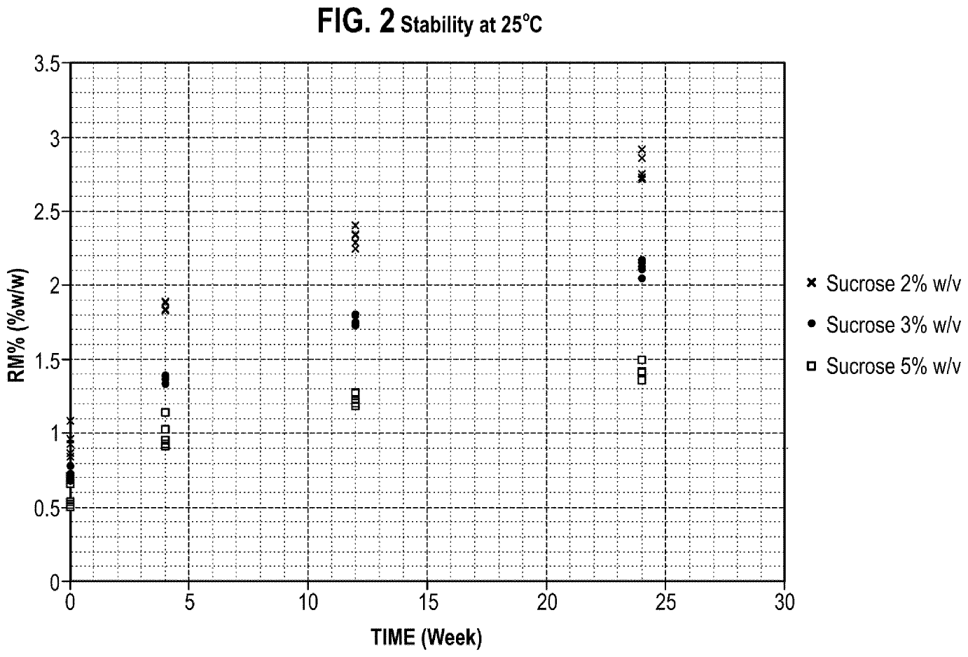

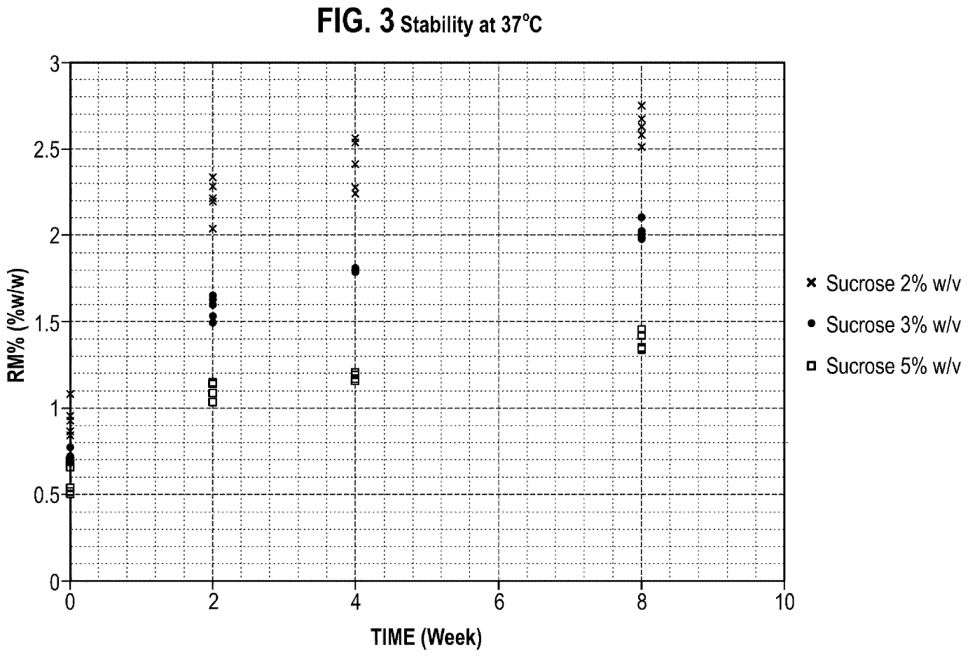

RM % was determined in the lyophilized product stored at 2-8° C., at time zero and at different times up to 2.5 years from the production day (results in FIG. 1). RM % was also determined in the lyophilized product incubated at 25° C./60% RH for times up to 6 months (results in FIG. 2), and in the lyophilized product incubated at 37° C./75% RH for times up to 2 months (results in FIG. 3).

The Karl Fisher method was used to determine RM % in the lyophilized cakes with a colorimetric Karl Fisher Titrator with Oven Metrohm 831 under the following conditions:
Oven temperature: 110° C.
Nitrogen flow rate: 60 ml/min
Stop drift: 10 μg/min

The three lyophilized products, produced from pre-lyo bulks Formulations 0, 1 and 2, having different sucrose concentrations, were all within the 3% RM specification limit up to the end of both accelerated stability tests at the different temperatures 37° C. and 25° C.

Aggregation % was determined by SE-UPLC in the lyophilized product of formulations 1 and 2 at time zero and at several subsequent times up to 6 months, at 37° C. and 25° C./60% RH, respectively, and the results obtained are summarized in the following Table 2. No significant variation in the aggregation percentages was observed amongst the three different formulations tested and, also under stability conditions, no increasing aggregation trend was observed over time for the pre-lyo formulations of this invention.

TABLE 2
Aggregation % under accelerated stabilities conditions on freeze-dried
products from pre-lyo formulation with different sucrose concentration

| | Sucrose | | | | | | |
|---|---|---|---|---|---|---|---|
| | concentration in | Time points | | | | | |
| Stability | pre-lyo bulk | Time | 2 | 4 | 2 | 3 | 6 |
| conditions | (% w/v) | zero | weeks | weeks | months | months | months |
| 37° C. | 3 | 5.1 | 3.2 | 5.1 | 3.6 | | |
| | 5 | 5.9 | 6.1 | 6.2 | 4.7 | | |
| 25° C./60% | 3 | 5.1 | | 3.6 | | 4.0 | 5.1 |
| RH | 5 | 5.9 | | 4.4 | | 5.1 | 5.0 |

Example 2: Preparation of MenB antigens composition in liquid formulation, of a MenABCW135Y reconstituted vaccine and related tests

A MenB antigens composition including the Bexsero protein antigens in amount of 100 μg/ml, and the OMV component in amount of 50 μg/ml, has been prepared in the same liquid formulation of the Bexsero® product added with Alum as adsorbing agent for the protein antigens, in order to verify the behavior of the MenB antigens composition when used for reconstitution of the present MenACW135Y composition.

This liquid formulation has been prepared in three batches, for the subsequent reconstitution of the MenACW135Y composition in the 2 different solid formulations of Example 1, Formulation 1 and Formulation 2 of Table 1, that differ for the amount of bulking agent in the pre-lyophilization bulk.

Starting from these compositions, a MenABCW135Y reconstituted vaccine was prepared, for each dose to be delivered to correspond to a volume of 0.5 ml.

Appropriate procedures have been defined to guarantee that the lyophilized product is reconstituted with the required volume of liquid formulation to obtain the target dosage of 0.5 ml for injection. Reconstitution and injection procedures have been designed to guarantee the dose injection for all the configurations defined for the target dose, using a pre-filled syringe containing the Men B liquid formulation, a vial containing the lyophilized Men ACWY formulation, a sterile disposable needle for both the reconstitution/withdrawing step and for the injection to the subject.

Standard sanitary practices for contamination prevention during reconstitution and withdrawing procedures need to be followed to prevent product contamination at use. The procedure followed included the following steps: the pre-filled syringe, equipped with the needle, was inserted into the rubber stopper of the vial; the contents of the syringe was then expelled into the vial to reconstitute the lyophilized material therein, and the contents of the vial was finally withdrawn back into the syringe.

On this reconstituted vaccine composition, stability tests were conducted, in particular to verify adsorption onto Alum of MenB protein antigens after reconstitution.

The percent adsorption of Bexsero antigens to Alum was indirectly measured by analyzing the supernatant of the vaccine after centrifugation to detect the amount of unbound antigen by SDS-PAGE. In the SDS-PAGE analysis, the proteins present in the supernatant after centrifugation are dissociated into their polypeptide chains by sodium dodecyl sulphate (SDS), after reduction of the disulphide bonds. The reduced proteins are then separated via polyacrylamide gel electrophoresis (PAGE). Bands are visualized with Coomassie Blue. The level of adsorption is quantified by inspection, comparing the band intensity of un-adsorbed antigen to known quantities of reference standard antigens, and the amount of adsorbed antigen is determined by subtraction (i.e. Percentage of adsorption=100%-percent of protein in vaccine supernatant). For this compatibility exercise, the extent of antigen adsorption was determined immediately after (T=0) and 24 hours following reconstitution (T=24; storage at 2-8° C.). As a reference standard of intensity in the electrophoretic pattern in this study was taken the intensity corresponding to 10% of non-adsorbed antigen (i.e. adsorption ≥90%).

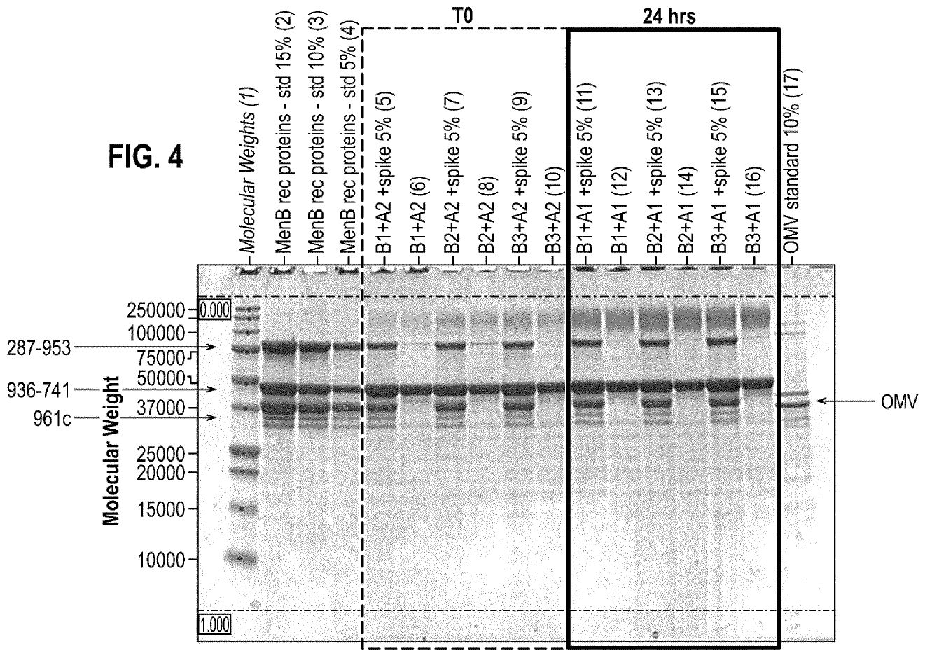

Results are reported in the FIG. 4 annexed and indicate no significant desorption of the 287-953, 961c and OMV antigens occurs after reconstitution of the MenACW135Y lyophilized component with liquid MenB component. This is visually evident from the absence of antigen specific bands in the supernatant fractions loaded onto the gels. The % of non-adsorbed 936-741 antigen was approximately 10%, while the adsorption of the other 3 MenB antigens was nearly 100%.

Studies by Reverse-Phase-UPLC have been also carried out to estimate the amount of not adsorbed MenB antigens, in particular of the 936-741 antigen, on Alum after reconstitution of each vial of the lyophilized tetravalent MenACW135Y vaccine component with 600 ml of Bexsero vaccine.

After centrifugation for 20 minutes at 2100×g at 20° C., the supernatant of the reconstituted MenACW135Y vaccine was analysed by RP-UPLC, to determine the amounts of unbound recombinant MenB antigens and the PorA and PorB Outer Membrane Proteins (OMPs) residing within the OMV.

The RP-UPLC analysis was performed using a Waters Acquity Hclass Bio UPLC System, coupled with a photodiode array detector (PDA) having an analytical flow cell. EMPOWER Waters chromatography software version 3 was used to program runs and to perform data analysis.

An Acquity BEH300 C4, 1.7 μm, 2.1×150 mm column was used at 60° C. while the samples were maintained in the autosampler at 6° C. The mixtures of MenB proteins were eluted by increasing the concentration of organic solvent 90% ACN in H₂O from 34% to 75% for 4 min. Both solvents were prepared with 0.1% trifluoracetic acid (TFA) was also used to improve the chromatographic peak shape.

The concentration of each antigen was extrapolated from the respective calibration curve built by analysis of a mixture of the four reference drug substance bulks (mock standard solution), injected in duplicate, before and after the samples.

The result was expressed as the average of two replicates prepared independently. During the development of the UPLC method, the only quantifiable protein in the supernatant detected prior to and just after reconstitution was the 936-741 protein, while the other MenB antigens have not been detected after reconstitution. Consequently, only this antigen was then quantified as a reference standard used to build the calibration curve and the results of RP-UPLC analyses therefore focus on the quantification of 936-741 protein only.

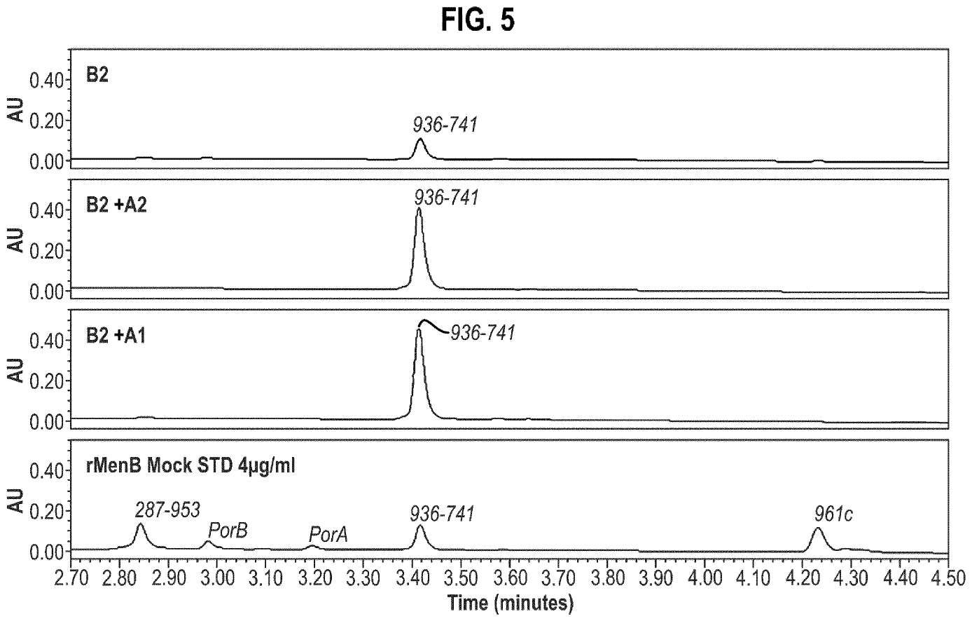

A representative RP-UPLC chromatogram of Bexsero lot B2 before (B2) and immediately after reconstitution (T=0) with MenACWY Lyo lots A1 (B2+A1) and A2 (B2+A2) is shown in FIG. 5. Both lots A1 and A2 contribute with 12.5 mg of sucrose in the reconstituted composition.

Comparable results were observed for chromatograms run on reconstituted vaccines held at 2-8° C. for 24 hours. The extent of increased desorption of 936-741 upon reconstitution is illustrated by the increased signal originating from the combined vaccine lot samples (B2+A1 and B2+A2 traces) relative to the starting MenB B2 lot (B2 trace).

The trace “rMenB mock Std” represents a mock control of all antigens in solution. The absence of any visible peaks representing the elution of 287-953, 961c antigens or the PorA and PorB OMPs from OMV are indicative of the virtually complete adsorption of these proteins onto Alum before and after reconstitution.

Effect of the Bulking Agent in the MenACWY Solid Composition on the Absorption of MenB Antigens After Reconstitution with a Bexsero Liquid Formulation

A MenACWY lyophilized composition containing only the matrix of excipients, without antigens, was prepared with two different amounts of sucrose as bulking agent in the pre-lyophilization solution, 3% and 5% by weight with respect of the total volume of the solution.

A Bexsero liquid formulation was then used to reconstitute the MenACWY lyophilized placebo, and the amount of unadsorbed 936-741 onto Alum was evaluated by the same methods described above.

It was thus observed by inventors that with both amounts of sucrose tested the 936-741 antigen unadsorbed onto Alum is around 10% and tends to decrease over time, from time 0 until time of 1 months from reconstitution, especially for samples at 25° C. Moreover, a clear trend of decrease of unadsorbed Men antigen was observed by lowering the amount of sucrose in the lyophilized MenACWY composition from 5% w/v to 3% w/v, as shown by the results in the Tables 3 and 4 below.

TABLE 3
Not adsorption % of 936-741 onto Alum after reconstitution
with MenACWY placebo with 5% w/v sucrose

| | Time 0 | | 1 week | | 2 weeks | | 1 month | |
|---|---|---|---|---|---|---|---|---|
| Sample | Average | CV (%) | Average | CV (%) | Average | CV (%) | Average | CV (%) |
| MenB formulation | — | — | 15.54 | 0.10 | 15.37 | 0.20 | 14.22 | 0.76 |
| (2-8° C.) | | | | | | | | |
| MenB formulation | — | — | 12.69 | 0.15 | 11.01 | 0.20 | 9.52 | 0.51 |
| (25° C.) | | | | | | | | |

TABLE 4
Not adsorption % of 936-741 onto Alum after reconstitution
with MenACWY placebo with 3% w/v sucrose

| | Time 0 | | 1 week | | 2 weeks | | 1 month | |
|---|---|---|---|---|---|---|---|---|
| Sample | Average | CV (%) | Average | CV (%) | Average | CV (%) | Average | CV (%) |
| MenB formulation | — | — | — | — | 12.98 | 0.17 | 13.93 | 0.83 |
| (2-8° C.) | | | | | | | | |
| MenB formulation | — | — | — | — | 9.03 | 0.24 | 8.29 | 0.63 |
| (25° C.) | | | | | | | | |

### Example 3: Reconstitution of the MenACW135Y Solid Composition with a MenB Liquid Formulation and Effect on the Absorption of MenB Antigens after Reconstitution

The lyophilized composition of MenACW135Y antigens of this invention, as described above, was also tested after reconstitution with a liquid formulation of an improved immunogenic composition against serogroup B meningococcus, disclosed in WO 2020/030782 and indicated herein as heptavalent MenB composition after the fact that seven MenB antigens are contained therein, as described above in detail.

For the avoidance of doubt, the MenB component of the reconstituted vaccine was comprised in this case of all the BEXSERO vaccine antigens, together with an additional fHbp fusion protein, corresponding to the 231.13 fusion protein identified above as SEQ ID NO: 49, also indicated here as 741 231.13.

The three pre-lyophilized Formulations 0, 1 and 2 of Example 1 having respectively 2% w/v, 3% w/V and 5% w/v of sucrose have been lyophilized. Each of them was split into two batches and reconstituted with two MenB liquid heptavalent formulations differing from each other for the concentration of the 231.13 fHbp antigen, respectively amounting to 100 mg/ml and 400 mg/ml. The same two MenB heptavalent formulations as such were also used as a control.

The percentage of adsorption onto Alum of the MenB antigens was analyzed by RP-UPLC for each of the antigens according to the same methodology described above in Example 3. After centrifugation for 20 minutes at 2100×g, the supernatant of the reconstituted MenABCWY vaccine was analysed by RP-UPLC, in order to determine the amounts of unbound recombinant MenB antigens and the PorA protein residing within the OMVs.

TABLE 5
Concentration of MenB antigens of the heptavalent composition not adsorbed onto Alum, quantified
by RP-UPLC after reconstitution of MenACWY having different amounts of sucrose

| | 741 231.13 | Sucrose | 936-741 | 287-953 | 741 231.13 | 961c | PorA |
|---|---|---|---|---|---|---|---|
| | concentration | concentration in | unadsorbed | unadsorbed | unadsorbed | unadsorbed | unadsorbed |
| | in MenB | MenACWY pre-lyo | concentration | concentration | concentration | concentration | concentration |
| | (μg/ml) | solution (% w/v) | (μg/ml) | (μg/ml) | (μg/ml) | (μg/ml) | (μg/ml) |
| MenABCWY | 100 | 2 | 0.4* | 0.4* | 0.5* | 0.0* | 0.0* |
| | 400 | 2 | 1.2 | 0.5* | 1.1 | 0.0* | 0.2* |
| | 100 | 3 | 0.4* | 0.4* | 0.6* | 0.0* | 0.1* |
| | 400 | 3 | 1.1 | 0.5* | 0.8* | 0.0* | 0.2* |
| | 100 | 5 | 0.4* | 0.4* | 0.6* | 0.0* | 0.0* |
| | 400 | 5 | 1.2 | 0.5* | 0.7* | 0.0* | 0.1* |
| MenB | 100 | — | 0.2* | 0.5* | 0.5* | 0.0* | 0.0* |
| (control | 400 | — | 0.4* | 0.4* | 0.6* | 0.0* | 0.0* |
| groups) | | | | | | | |

*Out of calibration curve (<1)

The results obtained show that the MenACW135Y solid composition of this invention, at different concentrations of the bulking agent in the solid matrix of excipients of this composition, do not reflect in a different absorption of the MenB antigens onto Alum. Very surprisingly, the addition of a number of further polysaccharide antigens that are the MenACW135Y antigens and a further MenB antigen with respect to the BEXSERO formulation of Example 3, does not affect significantly the amount of unadsorbed 936-741 protein.

### CONCLUSIONS

As shown by the results in the experimental part above, the solid composition of the lyophilized MenACW135Y antigens with the matrix of excipients above described, when reconstituted with liquid formulations of Men B antigens, meets the relevant quality requirements for the final vaccine, in particular in terms of long term stability of the lyophilized product, and also surprisingly minimizes the MenB antigens desorption from the adsorbing agent following reconstitution, which is particularly surprising for a composition with so many antigen components.

Moreover, it is a consolidated assumption in vaccines manufacturing that the formulation composition of a Drug Product should be kept as simple as possible. The addition of excipients other than buffering and tonicity agents is considered acceptable only if it is necessary to stabilize the antigens or significantly improve vaccine delivery or manufacturing. Any excipient added in the final product should be therefore justified.

The bulking agent excipient, in particular sucrose, for the MenACWY lyophilized composition of the MenABCWY vaccine product according to this invention has been demonstrated to be a suitable bulking agent: an optimal concentration range has been found to ensure a good stability profile of the lyophilized components over its shelf-life while minimizing the desorption from Al(O)OH of Men B antigens, in particular of 936-741 fusion protein, upon reconstitution of lyophilized composition with the MenB liquid composition.

1. An immunogenic composition against N. meningitidis serogroups A, C, W135, and Y, comprising a composition of capsular polysaccharide antigens of the meningococcal serogroups A, C, W135, and Y conjugated to carrier protein(s) and one or more pharmaceutically acceptable excipients, wherein said composition is a lyophilized composition and said excipients comprise one or more bulking agents in amount ≤30 mg per unit dose of the composition of polysaccharide antigens in lyophilized form.

2. The immunogenic composition of claim 1, wherein said bulking agent is in amount ≤25 mg per unit dose of the composition of capsular polysaccharide antigens in lyophilized form.

3. The immunogenic composition of claim 1, wherein said bulking agent is selected from the group consisting of sucrose, mannitol, trehalose, and mixtures thereof.

4. The immunogenic composition of claim 1, wherein said bulking agent is sucrose.

5. (canceled)

6. (canceled)

7. The immunogenic composition of claim 1, wherein the amount of said one or more bulking agents is 12.5 mg per unit dose of the composition of polysaccharide antigens in lyophilized form.

8. (canceled)

9. The immunogenic composition of claim 1, wherein said capsular polysaccharide antigens of the meningococcal serogroups A, C, W135, and Y are conjugated to CRM₁₉₇ as carrier protein.

10. A pre-lyophilization solution comprising a composition of capsular polysaccharide antigens of the meningococcal serogroups A, C, W135, and Y conjugated to carrier protein(s), one or more pharmaceutically acceptable excipients, and an aqueous solution of a buffering agent, wherein said excipients comprise one or more bulking agents in a concentration ≤6% w/v, optionally ≤5% w/v with respect to the total volume of the solution.

11. The pre-lyophilization solution of claim 10, wherein the concentration of said bulking agent ranges from 6% w/v to 2% w/v, optionally from 5% w/v to 2% w/v, or it is 3% w/v with respect to the total volume of the pre-lyophilized solution.

12. The pre-lyophilization solution of claim 10, wherein said aqueous solution of a buffering agent is a phosphate buffer comprising potassium monobasic phosphate, to which potassium dibasic phosphate is added.

13. A reconstituted vaccine against N. meningitidis serogroups A, B, C, W135, and Y comprising the immunogenic composition against N. meningitidis serogroups A, C, W135, and Y in lyophilized form as defined in claim 1, reconstituted with a liquid formulation of an immunogenic composition against N. meningitidis serogroup B comprising one or more Men B antigens and an adsorbing agent for said antigens.

14. The reconstituted vaccine of claim 13, wherein said immunogenic composition against N. meningitidis serogroup B comprises a meningococcal NHBA antigen, a meningococcal NadA antigen, a meningococcal fHbp antigen, a meningococcal outer membrane vesicles (OMVs), and a fusion polypeptide comprising v1, v2 and v3 meningococcal fHbp polypeptides in the order v2-v3-v1 from N- to C-terminus.

15. The reconstituted vaccine of claim 14, wherein the v1 fHbp polypeptide is a mutant v1.13 fHbp polypeptide comprising an amino acid sequence having at least 80% sequence identity to SEQ ID NO: 16 wherein the amino acid sequence includes a substitution mutation at one or more of residues S216, E211 or E232 of SEQ ID NO: 16.

16. The reconstituted vaccine of claim 14, wherein the mutant v1.13 meningococcal fHbp polypeptide comprises an amino acid sequence that differs from SEQ ID NO: 16 by at least one or more of the substitutions S216R, E211A and E232A.

17. (canceled)

18. The reconstituted vaccine of claim 14, wherein said meningococcal NHBA antigen and said meningococcal fHbp antigen are fusion proteins with meningococcal accessory proteins, e.g. a NHBA-GNA1030 fusion protein and a GNA2091-fHbp fusion protein.

19. The reconstituted vaccine of claim 13, wherein said liquid formulation is an aqueous solution comprising sodium chloride at a concentration ≤3.8 mg/ml, preferably of 2.8 mg/ml.

20. (canceled)

21. The reconstituted vaccine of claim 13, wherein said adsorbent agent comprises a compound containing aluminium selected from aluminium hydroxide, aluminium salts and mixtures thereof; and preferably is Alum.

22. A kit comprising (i) a first container (e.g. a prefilled syringe) comprising the immunogenic composition against N. meningitidis serogroup B in a liquid form as defined in claim 13; and (ii) a second container (e.g. a vial) comprising an immunogenic composition against N. meningitidis serogroups A, C, W135, and Y, in a solid lyophilized form as defined in claim 1, wherein said first container and said second container are containers separated from each other or together form a multi-container.

23. (canceled)

24. (canceled)

25. (canceled)

26. A method for preparing the reconstituted vaccine against N. meningitidis serogroups A, B, C, W135, and Y of claim 13, comprising a step of reconstituting said immunogenic composition against N. meningitidis serogroups A, C, W135, and Y in lyophilized form as defined in claim 1, with a liquid formulation of an immunogenic composition against N. meningitidis serogroup B comprising one or more Men B antigens and an adsorbing agent for said Men B antigens, as defined in claim 13.

27. A method for the treatment or prevention of infection and disease caused by N. meningitidis in a subject in need thereof comprising administering to said subject a therapeutically effective amount of an immunogenic composition according to claim 1, or of a reconstituted vaccine according to claim 13.

28. A method of inducing an immune response to N. meningitidis in a subject, the method comprising administering a therapeutically or prophylactically effective amount of an immunogenic composition according to claim 1 or of a reconstituted vaccine according to claim 13.

29. (canceled)


SEQUENCE LISTING (56 sequences)

SEQ ID NO: 1 - protein, 255 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 2 - protein, 254 aa, Neisseria meningitidis
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 3 - protein, 262 aa, Neisseria meningitidis
CSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 4 - protein, 488 aa, Neisseria meningitidis
MFKRSVIAMACIFALSACGGGGGGSPDVKSADTLSKPAAPVVSEKETEAKEDAPQAGSQGQGAPSAQGSQDMAAVSEENTGNGGAVTADNPKNEDEVAQNDMPQNAAGTDSSTPNHTPDPNMLAGNMENQATDAGESSQPANQPDMANAADGMQGDDPSAGGQNAGNTAAQGANQAGNNQAAGSSDPIPASNPAPANGGSNFGRVDLANGVLIDGPSQNITLTHCKGDSCSGNNFLDEEVQLKSEFEKLSDADKISNYKKDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPAKGEMLAGAAVYNGEVLHFHTENGRPYPTRGRFAAKVDFGSKSVDGIIDSGDDLHMGTQKFKAAIDGNGFKGTWTENGSGDVSGKFYGPAGEEVAGKYSYRPTDAEKGGFGVFAGKKEQD

SEQ ID NO: 5 - protein, 364 aa, Neisseria meningitidis
MSMKHFPSKVLTTAILATFCSGALAATSDDDVKKAATVAIVAAYNNGQEINGFKAGETIYDIGEDGTITQKDATAADVEADDFKGLGLKKVVTNLTKTVNENKQNVDAKVKAAESEIEKLTTKLADTDAALADTDAALDETTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKADIAKNSARIDSLDKNVANLRKETRQGLAEQAALSGLFQPYNVGRFNVTAAVGGYKSESAVAIGTGFRFTENFAAKAGVAVGTSSGSSAAYHVGVNYEW

SEQ ID NO: 6 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 7 - protein, 434 aa, synthetic construct
  REGION 1..434: note = Fusion protein of fHbp
MVSAVIGSAAVGAKSAVDRRTTGAQTDDNVMALRIETTARSYLRQNNQTKGYTPQISVVGYDRHLLLLGQVATEGEKQFVGQIARSEQAAEGVYNYITVASLPRTAGDIAGDTWNTSKVRATLLGISPATRARVKIVTYGNVTYVMGILTPEEQAQITQKVSTTVGVQKVITLYQNYVQRGSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 8 - protein, 468 aa, Neisseria meningitidis
SPDVKSADTLSKPAAPVVSEKETEAKEDAPQAGSQGQGAPSAQGGQDMAAVSEENTGNGGAAATDKPKNEDEGAQNDMPQNAADTDSLTPNHTPASNMPAGNMENQAPDAGESEQPANQPDMANTADGMQGDDPSAGGENAGNTAAQGTNQAENNQTAGSQNPASSTNPSATNSGGDFGRTNVGNSVVIDGPSQNITLTHCKGDSCSGNNFLDEEVQLKSEFEKLSDADKISNYKKDGKNDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPSKGEMLAGTAVYNGEVLHFHTENGRPSPSRGRFAAKVDFGSKSVDGIIDSGDGLHMGTQKFKAAIDGNGFKGTWTENGGGDVSGKFYGPAGEEVAGKYSYRPTDAEKGGFGVFAGKKEQD

SEQ ID NO: 9 - protein, 644 aa, synthetic construct
  REGION 1..644: note = Fusion protein of NHBA
MASPDVKSADTLSKPAAPVVSEKETEAKEDAPQAGSQGQGAPSAQGGQDMAAVSEENTGNGGAAATDKPKNEDEGAQNDMPQNAADTDSLTPNHTPASNMPAGNMENQAPDAGESEQPANQPDMANTADGMQGDDPSAGGENAGNTAAQGTNQAENNQTAGSQNPASSTNPSATNSGGDFGRTNVGNSVVIDGPSQNITLTHCKGDSCSGNNFLDEEVQLKSEFEKLSDADKISNYKKDGKNDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPSKGEMLAGTAVYNGEVLHFHTENGRPSPSRGRFAAKVDFGSKSVDGIIDSGDGLHMGTQKFKAAIDGNGFKGTWTENGGGDVSGKFYGPAGEEVAGKYSYRPTDAEKGGFGVFAGKKEQDGSGGGGATYKVDEYHANARFAIDHFNTSTNVGGFYGLTGSVEFDQAKRDGKIDITIPVANLQSGSQHFTDHLKSADIFDAAQYPDIRFVSTKFNFNGKKLVSVDGNLTMHGKTAPVKLKAEKFNCYQSPMAKTEVCGGDFSTTIDRTKWGVDYLVNVGMTKSVRIDIQIEAAKQ

SEQ ID NO: 10 - protein, 327 aa, Neisseria meningitidis
ATNDDDVKKAATVAIAAAYNNGQEINGFKAGETIYDIDEDGTITKKDATAADVEADDFKGLGLKKVVTNLTKTVNENKQNVDAKVKAAESEIEKLTTKLADTDAALADTDAALDATTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKDNIAKKANSADVYTREESDSKFVRIDGLNATTEKLDTRLASAEKSIADHDTRLNGLDKTVSDLRKETRQGLAEQAALSGLFQPYNVG

SEQ ID NO: 11 - protein, 261 aa, Neisseria meningitidis
CSSGSGSGGGGVAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ

SEQ ID NO: 12 - protein, 260 aa, Neisseria meningitidis
CSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQ

SEQ ID NO: 13 - protein, 168 aa, Neisseria meningitidis
ATYKVDEYHANARFAIDHFNTSTNVGGFYGLTGSVEFDQAKRDGKIDITIPIANLQSGSQHFTDHLKSADIFDAAQYPDIRFVSTKFNFNGKKLVSVDGNLTMHGKTAPVKLKAEKFNCYQSPMEKTEVCGGDFSTTIDRTKWGMDYLVNVGMTKSVRIDIQIEAAKQ

SEQ ID NO: 14 - protein, 179 aa, Neisseria meningitidis
VSAVIGSAAVGAKSAVDRRTTGAQTDDNVMALRIETTARSYLRQNNQTKGYTPQISVVGYNRHLLLLGQVATEGEKQFVGQIARSEQAAEGVYNYITVASLPRTAGDIAGDTWNTSKVRATLLGISPATQARVKIVTYGNVTYVMGILTPEEQAQITQKVSTTVGVQKVITLYQNYVQR

SEQ ID NO: 15 - protein, 255 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQ

SEQ ID NO: 16 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQ

SEQ ID NO: 17 - protein, 248 aa, synthetic construct
  REGION 1..248: note = MUTATED fhbp POLYPEPTIDE
VAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDAKGSYSLGIFGGQAQEVAGSAAVETANGIHHIGLAAKQ

SEQ ID NO: 18 - protein, 248 aa, synthetic construct
  REGION 1..248: note = MUTATED fhbp POLYPEPTIDE
VAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDAKGSYRLGIFGGQAQEVAGSAEVETANGIHHIGLAAKQ

SEQ ID NO: 19 - protein, 263 aa, Neisseria meningitidis
CSSGGGGSGGGGVAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQ

SEQ ID NO: 20 - protein, 251 aa, Neisseria meningitidis
VAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQ

SEQ ID NO: 21 - protein, 251 aa, synthetic construct
  REGION 1..251: note = MUTATED fhbp POLYPEPTIDE
VAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAEKGSYRLGIFGGQAQEVAGSAEVETANGIRHIGLAAKQ

SEQ ID NO: 22 - protein, 251 aa, synthetic construct
  REGION 1..251: note = MUTATED fhbp POLYPEPTIDE
VAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAAKGSYRLGIFGGQAQEVAGSAEVETANGIRHIGLAAKQ

SEQ ID NO: 23 - protein, 251 aa, synthetic construct
  REGION 1..251: note = MUTATED fhbp POLYPEPTIDE
VAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAAKGSYSLGIFGGQAQEVAGSAAVETANGIRHIGLAAKQ

SEQ ID NO: 24 - protein, 273 aa, Neisseria meningitidis
MNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 25 - protein, 254 aa, Neisseria meningitidis; identical to SEQ ID NO: 2

SEQ ID NO: 26 - protein, 247 aa, Neisseria meningitidis
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 27 - protein, 281 aa, Neisseria meningitidis
MNRTAFCCLSLTTALILTACSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 28 - protein, 262 aa, Neisseria meningitidis; identical to SEQ ID NO: 3

SEQ ID NO: 29 - protein, 250 aa, Neisseria meningitidis
VAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 30 - protein, 247 aa, synthetic construct
  REGION 1..247: note = MUTATED fhbp POLYPEPTIDE
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 31 - protein, 250 aa, synthetic construct
  REGION 1..250: note = MUTATED fhbp POLYPEPTIDE
VAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 32 - protein, 757 aa, synthetic construct
  REGION 1..757: note = MUTATED fhbp POLYPEPTIDE
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDAKGSYSLGIFGGQAQEVAGSAAVETANGIHHIGLAAKQ

SEQ ID NO: 33 - protein, 757 aa, synthetic construct
  REGION 1..757: note = MUTATED fhbp POLYPETIDE
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDAKGSYRLGIFGGQAQEVAGSAEVETANGIHHIGLAAKQ

SEQ ID NO: 34 - protein, 760 aa, synthetic construct
  REGION 1..760: note = MUTATED fhbp POLYPEPTIDE
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAEKGSYRLGIFGGQAQEVAGSAEVETANGIRHIGLAAKQ

SEQ ID NO: 35 - protein, 760 aa, synthetic construct
  REGION 1..760: note = MUTATED fhbp POLYPEPTIDE
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAAKGSYRLGIFGGQAQEVAGSAEVETANGIRHIGLAAKQ

SEQ ID NO: 36 - protein, 760 aa, synthetic construct
  REGION 1..760: note = MUTATED fhbp POLYPEPTIDE
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAAKGSYSLGIFGGQAQEVAGSAAVETANGIRHIGLAAKQ

SEQ ID NO: 37 - protein, 256 aa, synthetic construct
  REGION 1..256: note = fHbp polypeptide with His6 tag
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELGLAAKQLNVDLAAADIKPDGKRHAVISGSVLYNQAEKGSYSLGIFGGKAQEVAGSAEVKTVNGIRHLEHHHHHH

SEQ ID NO: 38 - protein, 256 aa, synthetic construct
  REGION 1..256: note = fHbp polypeptide with His6 tag
VAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQLEHHHHHH

SEQ ID NO: 39 - protein, 256 aa, synthetic construct
  REGION 1..256: note = Mutated fHbp polypeptide
VAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDAKGSYSLGIFGGQAQEVAGSAEVETANGIHHIGLAAKQLEHHHHHH

SEQ ID NO: 40 - protein, 256 aa, synthetic construct
  REGION 1..256: note = Mutated fHbp polypeptide
VAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYRLGIFGGQAQEVAGSAEVETANGIHHIGLAAKQLEHHHHHH

SEQ ID NO: 41 - protein, 259 aa, synthetic construct
  REGION 1..259: note = fHbp polypeptide with His6 tag
VAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQLEHHHHHH

SEQ ID NO: 42 - protein, 259 aa, synthetic construct
  REGION 1..259: note = Mutated fHbp polypeptide with His6 tag
VAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAAKGSYSLGIFGGQAQEVAGSAEVETANGIRHIGLAAKQLEHHHHHH

SEQ ID NO: 43 - protein, 753 aa, synthetic construct
  REGION 1..753: note = fHbp fusion polypeptide
VAADIGAGLADALTAPLDHKDKSLQSLTLDQSVRKNEKLKLAAQGAEKTYGNGDSLNTGKLKNDKVSFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVRKNEKLKLAAQGAEKTYGNGSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAAIKPDGKRHAVISGSVLYNQAEKGSYSLGIFGKAQEVAGSAEVKTVNGIRHIGLAAKQ

SEQ ID NO: 44 - protein, 753 aa, synthetic construct
  REGION 1..753: note = fHbp fusion polypeptide
VAADIGAGLADALTAPLDHKDKSLQSLTLDQVVRKNEKLKLAAQGAEKTYGNGDSLNTGKLKNDKVSFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVSKNEKLKLAAQGAEKTYGNGSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAAIKPDGKRHAVISGSVLYNQAEKGSYSLGIFGKAQEVAGSAEVKTVNGIRHIGLAAKQ

SEQ ID NO: 45 - protein, 274 aa, Neisseria meningitidis
MNRTAFCCFSLTAALILTACSSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQ

SEQ ID NO: 46 - protein, 282 aa, Neisseria meningitidis
MNRTTFCCLSLTAALILTACSSGGGGSGGGGVAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQ

SEQ ID NO: 47 - protein, 254 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 48 - protein, 12 aa, synthetic construct
  REGION 1..12: note = N-terminal sequence
MGPDSDRLQQRR

SEQ ID NO: 49 - protein, 769 aa, synthetic construct
  REGION 1..769: note = MUTATED fhbp POLYPETIDE
MGPDSDRLQQRRVAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDAKGSYRLGIFGGQAQEVAGSAEVETANGIHHIGLAAKQ

SEQ ID NO: 50 - protein, 4 aa, synthetic construct
GGSG

SEQ ID NO: 51 - protein, 5 aa, synthetic construct
GGSGG

SEQ ID NO: 52 - protein, 5 aa, synthetic construct
GSGSG

SEQ ID NO: 53 - protein, 5 aa, synthetic construct
GSGGG

SEQ ID NO: 54 - protein, 5 aa, synthetic construct
GGGSG

SEQ ID NO: 55 - protein, 5 aa, synthetic construct
GSSSG

SEQ ID NO: 56 - protein, 6 aa, synthetic construct
GSGGGG